(19) United States
(12) Patent Application Publication
(10) Pub. No.: US 2005/0287532 A9
(43) Pub. Date: Dec. 29, 2005

(54) Methods for monitoring drug activities in vivo

(76) Inventors: Michael E. Burczynski, Swampscott, MA (US); Natalie C. Twine, Goffstown, NH (US); Andrew J. Dorner, Lexington, MA (US); William L. Trepicchio, Andover, MA (US)

Correspondence Address: NIXON PEABODY, LLP, 401 9TH STREET, NW, SUITE 900, WASHINGTON, DC, 20004-2128, US

(21) Appl. No.: 10/775,169

(22) Filed: Feb. 11, 2004

Related U.S. Application Data
(60) Provisional application No. 60/446,133, filed on Feb. 11, 2003.
(60) Provisional application No. 60/459,782, filed on Apr. 3, 2003.
(60) Provisional application No. 60/538,246, filed on Jan. 23, 2004.

(51) Int. Cl. C12Q 1/68; G01N 33/567

(52) U.S. Cl. 435/6; 435/7.21

(57) ABSTRACT

Methods, systems and equipment useful for monitoring in vivo activities of CCI-779 or other drugs. Numerous drug activity genes can be identified by the present invention. The expression profiles of these genes in peripheral blood mononuclear cells can be modulated by CCI-779 or other drugs. Therefore, these genes can be used as surrogate markers for monitoring drug activities in vivo.

This application claims the benefit and incorporates by reference the entire disclosures of U.S. Provisional Application Serial No. 60/446,133, filed Feb. 11, 2003 and entitled “Methods for Monitoring Drug Activities in Vivo,” U.S. Provisional Application Serial No. 60/459,782, filed Apr. 3, 2003 and entitled “Methods for Diagnosing RCC and/or Solid Tumors,” and U.S. Provisional Application, filed Jan. 23, 2004 and entitled “Methods for Prognosis and Treatment of Solid Tumors” (by Michael Burczynski, et al.). In addition, this application incorporates by reference all materials recorded in compact discs labeled “Copy 1” and “Copy 2.” Each of the compact discs includes “Sequence Listing.ST25.txt” (3,647 KB, created Feb. 9, 2004) and “Table 1 (the Qualifier Table).txt” (210 KB, created Feb. 8, 2004).

### TECHNICAL FIELD

This invention relates to methods, systems and equipment useful for monitoring in vivo activities of CCI-779 or other drugs.

### BACKGROUND

CCI-779 is an ester analog of the immunosuppressant rapamycin and as such is a potent, selective inhibitor of the mammalian target of rapamycin. The mammalian target of rapamycin (mTOR) activates multiple signaling pathways, including phosphorylation of p70s6kinase, which results in increased translation of 5′ TOP mRNAs encoding proteins involved in translation and entry into the G1 phase of the cell cycle. By virtue of its inhibitory effects on mTOR and cell cycle control, CCI-779 may function as a cytostatic and immunosuppressive agent. CCI-779 has been used as an anticancer drug and is currently being evaluated for indications in clinical trials for treating various oncology and inflammatory diseases. These diseases include, but are not limited to, renal cell carcinoma (RCC), prostate cancer, gliomas, lung cancer, and non-Hodgkin's lymphoma.

### SUMMARY OF THE INVENTION

One of the main objectives of clinical pharmacogenomic studies is to identify suitable markers for monitoring in vivo activities of CCI-779 or other drugs. The present invention employs easily-obtained tissues, such as peripheral blood, as surrogate tissues for the detection of in vivo activities of CCI-779 or other drugs.

In one aspect, the present invention provides methods that are useful for detecting in vivo activities of CCI-779 or other drugs. The methods include comparing an expression profile of at least one drug activity gene in a peripheral blood sample of a patient of interest to a reference expression profile of the gene, where the gene is differentially expressed in peripheral blood mononuclear cells (PBMCs) of patients who have a non-blood disease and are subject to a drug therapy as compared to PBMCs isolated from the patients prior to the drug therapy. In many embodiments, the patient of interest has the non-blood disease and is being treated by the drug therapy.

In one embodiment, the drug therapy is an anti-cancer therapy, such as CCI-779 therapy, and the non-blood disease is a solid tumor, such as RCC, prostate cancer, or head/neck cancer. In another embodiment, the drug activity genes of the present invention are selected from Table 5.

The peripheral blood samples used in the present invention can be, without limitation, whole blood samples or samples comprising enriched PBMCs. Other peripheral blood samples that include PBMCs can also be employed in the present invention.

The expression profiles of the drug activity genes can be determined by various means, such as quantitative RT-PCR, Northern Blot, in situ hybridization, slot-blotting, nuclease protection assay, nucleic acid arrays, enzyme-linked immunosorbent assays (ELISAs), radioimmunoassay (RIAs), fluorescence-activated cell sorters (FACSs), or Western Blots. In addition, two-dimensional SDS-polyacrylamide gel electrophoresis and other high-through nucleic acid or protein detection techniques can also be used.

In many embodiments, the reference expression profile and the expression profile being compared are prepared using the same or comparable methodology. In one example, the reference expression profile is an average baseline expression profile of drug activity genes in peripheral blood samples isolated from patient or patients prior to a drug treatment. In another example, the reference expression profile is an expression profile of drug activity genes in peripheral blood samples of the patient of interest. The reference expression profile and the expression profile being compared can be obtained from peripheral blood samples isolated at different time points in a drug treatment.

In one embodiment, the drug activity genes used in the present invention are over-expressed (or under-expressed) in PBMCs of patient who have a non-blood disease as compared to PBMCs of humans who do not have the non-blood disease. The drug therapy being investigated can down-regulate (or up-regulate) the expression of the drug activity genes in PBMCs of patients who have the non-blood disease.

In another embodiment, the expression of the drug activity genes in PBMCs can be stimulated (or suppressed) by phytohemagglutinin (PHA). The drug therapy being investigated can down-regulate (or up-regulate) the expression of these genes in PHA-treated PBMCs.

In another aspect, the present invention provides other methods that are useful for detecting in vivo activities of CCI-779 or other drugs. The methods include comprises comparing an expression profile of at least one drug activity gene in a peripheral blood sample of a patient of interest to a reference expression profile of the gene, where the RNA transcript(s) of the drug activity gene can hybridize under stringent or nucleic acid array hybridization conditions to one or more qualifiers selected from the Qualifier Table.

In yet another aspect, the present invention provides methods useful for identifying drug activity genes. The methods include detecting the gene expression profile in peripheral blood samples of patients who have a non-blood disease and are subject to a drug therapy, and comparing the gene expression profile to a baseline gene expression profile in peripheral blood samples isolated before the drug therapy. Drug activity genes whose expression levels in peripheral blood samples can be modulated by the drug therapy can therefore be identified.

In still another aspect, the present invention provides kits useful for detecting in vivo activities of CCI-779 or other drugs. In one embodiment, the kits include a plurality of polynucleotides, and each polynucleotide can hybridize under stringent or nucleic acid array hybridization conditions to an RNA transcript, or the complement thereof, of a different respective drug activity gene. In another embodiment, the kits include a plurality of antibodies, and each antibody can bind to a polypeptide encoded by a different respective drug activity gene. The drug activity genes can be selected, without limitation, from Table 5.

In still yet another aspect, the present invention provides nucleic acid arrays useful for detecting in vivo activities of CCI-779 or other drugs. A substantial portion of all polypeptide probes on the nucleic acid array can hybridize under stringent or nucleic acid array hybridization conditions to RNA transcripts, or the complements thereof, of drug activity genes.

Other features, objects, and advantages of the present invention are apparent in the detailed description that follows. It should be understood, however, that the detailed description, while indicating preferred embodiments of the invention, are given by way of illustration only, not limitation. Various changes and modifications within the scope of the invention will become apparent to those skilled in the art from the detailed description.

### DETAILED DESCRIPTION

The present invention provides methods useful for the detection of in vivo activities of CCI-779 or other drugs. Numerous drug activity genes can be identified by the present invention. The expression profiles of these genes in PBMCs can be modulated by CCI-779 or other drugs. Accordingly, these genes can be used as surrogate markers for monitoring drug activities in vivo. In one embodiment, the methods of the present invention include comparing the expression profile of at least one drug activity gene in a peripheral blood sample of a patient of interest to a reference expression profile of the same drug activity gene. The patient of interest has a non-blood disease, such as RCC, prostate cancer, or another solid tumor, and is being treated by a drug therapy. A change in the peripheral blood expression profile of the drug activity gene is indicative of the in vivo activity of the drug therapy. In many cases, the reference expression profile can be determined by using baseline peripheral blood samples isolated from patients prior to the drug therapy. Peripheral blood samples amenable to the present invention include, but are not limited to, whole blood samples or samples comprising enriched PBMCs. Expression profiles of drug activity genes can be detected using a variety of methods, such as quantitative RT-PCT, Northern Blot, in situ hybridization, nucleic acid arrays, enzyme-linked immunosorbent assay (ELISA), radioimmunoassay (RIA), FACS (fluorescence-activated cell sorter), or Western Blot. The drug activity genes of the present invention may also be used for assessing the efficiency of a drug therapy.

Various aspects of the invention are described in further detail in the following sections. The use of sections is not meant to limit the invention. Each section and subsection may apply to any aspect of the invention. In this application, the use of “or” means “and/or” unless stated otherwise. Also, the use of the singular includes the plural unless stated otherwise.

A. General Methods for Identifying Drug Activity Genes

The availability of the human genome sequence, together with new developments in technology, such as DNA microarrays, proteomics and computational biology, allows systemic gene expression studies for various diseases. The present invention employs the systematic gene expression analysis technique to identify genes whose expression in peripheral blood can be modulated by a therapeutic agent such as CCI-779. These genes are herein referred to as “drug activity genes.” The genes whose expression levels in peripheral blood can be modified by CCI-779 are refereed to as “CCI-779 activity genes.”

Drug activity genes can be identified by comparing peripheral blood gene expression profiles before and after a drug treatment. Numerous methods are available for detecting and comparing gene expression profiles.

For instance, gene expression profiles can be detected by measuring the levels of RNA transcripts in peripheral blood samples. In one embodiment, total RNAs or polyA⁺ RNAs are isolated from peripheral blood samples using conventional means. The isolated RNAs can be amplified to produce cDNAs or cRNAs. Peripheral blood gene expression profiles can be determined by measuring the amount of the amplified cDNAs or cRNAs.

Peripheral blood gene expression profiles can also be determined by measuring the levels of polypeptides in peripheral blood samples. The amounts of polypeptides in peripheral samples can be detected using various methods, such as ELISAs, RIAs, FACSs, Western Blots or other immunoassays. In addition, two-dimensional gel electrophoresis/mass spectrometry or other high-throughput protein sequencing and identification methods can be used.

In one embodiment, nucleic acid arrays are used for detecting or comparing gene expression profiles in peripheral blood samples isolated at different stages of a therapeutic treatment. Nucleic acid arrays allow for quantitative detection of the expression levels of a large number of genes at one time. Examples of nucleic acid arrays include, but are not limited to, Genechip® microarrays from Affymetrix (Santa Clara, Calif.), cDNA microarrays from Agilent Technologies (Palo Alto, Calif.), and bead arrays described in U.S. Pat. Nos. 6,288,220 and 6,391,562.

The polynucleotides to be hybridized to nucleic acid arrays can be labeled with one or more labeling moieties to allow for detection of hybridized polynucleotide complexes. The labeling moieties can include compositions that are detectable by spectroscopic, photochemical, biochemical, bioelectronic, immunochemical, electrical, optical or chemical means. Exemplary labeling moieties include radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like. Unlabeled polynucleotides can also be employed. The polynucleotides can be DNA, RNA, or a modified form thereof.

Hybridization reactions can be performed in absolute or differential hybridization formats. In the absolute hybridization format, polynucleotides derived from one sample, such as a peripheral blood sample isolated from a cancer patient at a particular treatment stage, are hybridized to the probes in a nucleic acid array. Signals detected after the formation of hybridization complexes correlate to the polynucleotide levels in the sample. In the differential hybridization format, polynucleotides derived from two biological samples, such as one isolated from a cancer patient at a first stage of treatment and the other isolated from the same patient but at a second stage of treatment, are labeled with different labeling moieties. A mixture of these differently labeled polynucleotides is added to a nucleic acid array. The nucleic acid array is then examined under conditions in which the emissions from the two different labels are individually detectable. In one embodiment, the fluorophores Cy3 and Cy5 (Amersham Pharmacia Biotech, Piscataway N.J.) are used as the labeling moieties for the differential hybridization format.

Signals gathered from nucleic acid arrays can be analyzed using commercially available software, such as those provide by Affymetrix or Agilent Technologies. Controls, such as for scan sensitivity, probe labeling and cDNA/cRNA quantitation, can be included in the hybridization experiments. In many embodiments, the nucleic acid array expression signals are scaled or normalized before being subject to further analysis. For instance, the expression signals for each gene can be normalized to take into account variations in hybridization intensities when more than one array is used under similar test conditions. Signals for individual polynucleotide complex hybridization can also be normalized using the intensities derived from internal normalization controls contained on each array. In addition, genes with relatively consistent expression levels across the samples can be used to normalize the expression levels of other genes. In one embodiment, the expression levels of the genes are normalized across the samples such that the mean is zero and the standard deviation is one. In another embodiment, the expression data detected by nucleic acid arrays are subject to a variation filter which excludes genes showing minimal or insignificant variation across all samples.

A variety of peripheral blood samples can be used in the present invention. In one embodiment, the peripheral blood samples are whole blood samples. In another embodiment, the peripheral blood samples comprise enriched PBMCs. By “enriched,” it means that the percentage of PBMCs in the sample is higher than that in whole blood. In many cases, the PBMC percentage in an enriched sample is at least 1, 2, 3, 4, 5 or more times higher than that in whole blood. In many other cases, the PBMC percentage in an enriched sample is at least 90%, 95%, 98%, 99%, 99.5%, or more. Blood samples containing enriched PBMCs can be prepared using any method known in the art, such as Ficoll gradients centrifugation or CPTs (cell purification tubes).

Peripheral blood samples used in the present can be isolated at any stage of a drug treatment (including baseline samples isolated before the drug treatment). For instance, the samples can be isolated from patients at 1 day, 2 days, 3 days, 4 days, 5 days, 6 days, 1 week, 2 weeks, 3 weeks, 4 weeks, 8 weeks, or 16 weeks after initiation of a drug treatment. Other time points can also be used for isolating blood samples for monitoring or assessing in vivo drug activities.

In many embodiments, the patients being treated by a drug therapy of interest have a non-blood disease, such as a solid tumor. Solid tumors amenable to the present invention include, but are not limited, RCC, prostate cancer, head/neck cancer, ovarian cancer, testicular cancer, brain tumor, breast cancer, lung cancer, colon cancer, pancreas cancer, stomach cancer, bladder cancer, skin cancer, cervical cancer, uterine cancer, and liver cancer. In one embodiment, the solid tumors have the following characteristics: (1) a mass of hyperproliferating cells of clonal origin, and (2) acquisition of an aggressively invasive phenotype, where cancer cells leave the tissue of origin and establish new tumor metastases at distant sites. In one example, the patients have RCC.

Any cancer or disease treatment can be evaluated by the present invention. Exemplary cancer treatments include the use of cytokines, such as interferon or interleukin 2. In addition, chemotherapy drugs can be used, either individually or in combination with other drugs, cytokines or therapies. Suitable chemotherapy drugs include, but are not limited to, CCI-779, AN-238, vinblastine, floxuridine, 5-fluorouracil, and tamoxifen. AN238 is a cytotoxic agent which has 2-pyrrolinodoxorubicin linked to a somatostatin (SST) carrier octapeptide. AN238 can be targeted to SST receptors on the surface of RCC tumor cells. Moreover, monoclonal antibodies, antiangiogenesis drugs, and anti-growth factor drugs can be employed to treat cancers.

The gene expression profile in peripheral blood samples isolated at one stage of a drug treatment can be compared to that at another stage of the drug treatment. Drug activity genes that are differentially expressed in PBMCs at one stage of the treatment relative to another stage of treatment can therefore be identified. In one embodiment, the PBMC expression level of a drug activity gene is substantially higher at one stage than at another stage. For instance, an average PBMC expression level of a drug activity gene at one stage can be at least 1.5, 2, 3, 4, 5, 10, 20, or more times of that at another stage. In another embodiment, the PBMC expression level of a drug activity gene is substantially lower at one stage than at another stage. For instance, an average PBMC expression level of a drug activity gene at one stage can be no greater than 0.67, 0.5, 0.33, 0.25, 0.1, 0.05, or less times of that at another stage.

In yet another embodiment, drug activity genes can be identified using clustering algorithms based on the nucleic acid array gene expression data. For instance, unsupervised cluster analyses can be used to analyze and categorize genes which have differential expression patterns between different drug treatment stages. Algorithms for unsupervised cluster analysis include, but are not limited to, self-organized maps (SOMs), principle component analysis, average linkage clustering, and hierarchical clustering.

Supervised cluster analysis can also be used to organize and identify drug activity genes. Algorithms for supervised cluster analysis include, but are not limited to, nearest neighbors test, support vector machines, and SPLASH. Either two-class or multi-class correlation metrics can be used.

B. Identification of CCI-779 Activity Genes

In one embodiment, HG-U95Av2 gene chips (manufactured by Affymetrix) were used for detecting and comparing the levels of RNA transcripts in PBMC-enriched peripheral blood samples. Peripheral blood samples were isolated from RCC patients at different stages of CCI-779 treatment (See Example 1). The CCI-779 treatment included intravenous administration of 25, 75 or 250 mg of CCI-779 once weekly. Peripheral blood samples were isolated from RCC patients immediately before the initial administration of CCI-779, and then 8 weeks and 16 weeks thereafter.

cRNA were prepared from the isolated PBMC samples and hybridized to HG-U95Av2 genechips. Hybridization signals were collected for each oligonucleotide probe on the genechips. Signals from the oligonucleotide probes of the same qualifier were averaged. Qualifiers that produce different hybridization signals in samples isolated at different treatment time points were identified. Examples of these identified qualifiers are illustrated in Table 1 (“the Qualifier Table”).

In general, each qualifier in the Qualifier Table corresponds to at least one CCI-779 activity gene, and the RNA transcripts of the gene can hybridize under stringent or nucleic acid array hybridization conditions to the qualifier. As used herein, “hybridize to a qualifier” means to hybridize to at least one oligonucleotide probe of the qualifier. In many embodiments, the RNA transcripts of a CCI-779 activity gene can hybridize under stringent or nucleic acid array hybridization conditions to at least 2, 4, 6, 8, 10, 12, 14 or 16 oligonucleotide probes of the corresponding qualifier.

Table 2 lists the expression profiles of some qualifiers that produced different hybridization signals for PBMC samples isolated at different treatment stages. Each expression profile in Table 2 (“Baseline,” “8 wks Average,” or “16 wks Average”) was an average of PBMC samples of 110 RCC patients. Each expression profile under “Baseline,” “8 wks Average,” and “16 wks Average” represented the hybridization signals on the respective qualifier for PBMC samples isolated immediately before the initial CCI-779 administration, 8 weeks after the initial administration, and 16 weeks after the initial administration, respectively. The p-value of an ANOVA analysis was provided for each qualifier. The p-value suggests the statistical significance of the difference observed between the expression profiles obtained at different CCI-779 treatment stages. Lesser p-values indicate more statistical significance for the differences observed between different treatment stages.

TABLE 2
PBMC Gene Expression Profiles at
Different CCI-779 Treatment Stages

| | | 8 wks | 16 wks | | |
|---|---|---|---|---|---|
| | Baseline | Average | Average | ANOVA | |
| Qualifier | (n = 110) | (n = 110) | (n = 110) | P-Value | CPS |
| 34768_at | 7.90 | 19.4 | 20.8 | 2.5E−11 | 1 |
| 36097_at | 113 | 195 | 247 | 8.0E−11 | 2 |
| 36675_r_at | 62.2 | 109 | 144 | 8.4E−11 | 3 |
| 37940_f_at | 6.95 | 22.7 | 28.8 | 5.1E−10 | 4 |
| 34338_at | 16.0 | 29.3 | 35.4 | 5.9E−10 | 5 |
| 38991_at | 27.2 | 15.2 | 12.9 | 1.3E−09 | 6 |
| 38976_at | 42.6 | 103.7 | 139 | 4.6E−09 | 7 |
| 37905_r_at | 32 | 19.0 | 13.9 | 6.0E−09 | 8 |
| 38780_at | 11.9 | 23.1 | 32.0 | 7.1E−09 | 9 |
| 41215_s_at | 48.4 | 91 | 113 | 7.1E−09 | 10 |
| 35956_s_at | 11.1 | 3.81 | 4.19 | 9.3E−09 | 11 |
| 32332_at | 8.14 | 18.6 | 21.9 | 1.1E−08 | 13 |
| 41551_at | 8.48 | 14.0 | 19.3 | 1.8E−08 | 14 |
| 39332_at | 21.9 | 10 | 9.14 | 1.9E−08 | 15 |
| 40778_at | 6.19 | 11.7 | 15.6 | 1.9E−08 | 16 |
| 32232_at | 9.81 | 16.2 | 19.7 | 4.5E−08 | 17 |
| 34310_at | 11.3 | 19.1 | 26.3 | 5.8E−08 | 18 |
| 1184_at | 24.4 | 56.6 | 76.0 | 8.3E−08 | 19 |
| 35688_g_at | 10.1 | 20.2 | 15.7 | 8.4E−08 | 20 |
| 38981_at | 6.48 | 14.4 | 16.2 | 8.9E−08 | 21 |
| 37391_at | 173 | 89.3 | 28.4 | 1.3E−07 | 23 |
| 39593_at | 9.10 | 38.5 | 62.6 | 1.4E−07 | 24 |
| 32505_at | 8.62 | 4.62 | 4.24 | 1.6E−07 | 25 |
| AFFX- | 36.3 | 10.1 | 11.9 | 1.8E−07 | 26 |
| M27830_5_at | | | | | |
| 35666_at | 19.8 | 11.4 | 9.76 | 2.0E−07 | 27 |
| 39061_at | 18.6 | 37.0 | 47.0 | 2.1E−07 | 28 |
| 39748_at | 29.9 | 14.8 | 12.2 | 2.5E−07 | 29 |
| 40505_at | 12.8 | 26.1 | 37.0 | 2.7E−07 | 30 |
| 38597_f_at | 15.6 | 7.48 | 6.95 | 3.3E−07 | 31 |
| 39545_at | 13.0 | 34.2 | 43.8 | 3.3E−07 | 32 |
| 34268_at | 14.3 | 33.3 | 48.9 | 4.2E−07 | 33 |
| 38816_at | 13.3 | 7.05 | 5.62 | 4.8E−07 | 34 |
| 38732_at | 8.95 | 16.2 | 18.6 | 5.0E−07 | 35 |
| 40607_at | 9 | 23.8 | 36.4 | 5.1E−07 | 36 |
| 37200_at | 29.3 | 70.6 | 111 | 6.5E−07 | 37 |
| 283_at | 13.8 | 26.8 | 34.8 | 6.6E−07 | 38 |
| 40757_at | 10.8 | 33.0 | 56.1 | 7.1E−07 | 39 |
| 384_at | 19.2 | 36.0 | 51.7 | 9.0E−07 | 41 |
| 40814_at | 19 | 12.2 | 9.24 | 9.4E−07 | 42 |
| 37027_at | 31.8 | 60.0 | 85.0 | 9.4E−07 | 43 |
| 1760_s_at | 12.1 | 7.52 | 5.57 | 1.1E−06 | 44 |
| 1292_at | 16.3 | 37.4 | 39.8 | 1.1E−06 | 45 |
| 38287_at | 26.5 | 54.9 | 79.8 | 1.2E−06 | 46 |
| 41591_at | 10.9 | 6.90 | 4.76 | 1.3E−06 | 47 |
| 32475_at | 4.62 | 9.05 | 12.7 | 1.3E−06 | 48 |
| 40782_at | 36.4 | 23.9 | 8.29 | 1.4E−06 | 49 |
| 503_at | 19.7 | 33.3 | 43.2 | 1.5E−06 | 50 |
| 875_g_at | 132 | 68.7 | 10.5 | 1.5E−06 | 51 |
| 37185_at | 288 | 160 | 34.0 | 1.6E−06 | 52 |
| 32282_at | 10.9 | 4.57 | 4.95 | 1.7E−06 | 53 |
| 40981_at | 9.10 | 4.05 | 4.10 | 1.9E−06 | 54 |
| 34782_at | 74.0 | 46.2 | 24.6 | 1.9E−06 | 55 |
| 37373_at | 11.9 | 19.4 | 24.0 | 2.0E−06 | 56 |
| 40018_at | 8 | 4.67 | 3.57 | 2.1E−06 | 57 |
| 31638_at | 7.19 | 12.9 | 16.0 | 2.1E−06 | 58 |
| 38191_at | 14.4 | 3.14 | 3.38 | 2.4E−06 | 59 |
| 33396_at | 44.2 | 73.2 | 91.0 | 2.6E−06 | 60 |
| 38104_at | 4.33 | 6.76 | 8.76 | 2.6E−06 | 61 |
| 32193_at | 7.90 | 16.3 | 24.1 | 2.8E−06 | 62 |
| 38350_f_at | 4.90 | 8.95 | 10.4 | 2.8E−06 | 63 |
| 1985_s_at | 5.57 | 9.19 | 11.9 | 2.9E−06 | 64 |
| 38598_at | 10.5 | 3.71 | 3.05 | 3.3E−06 | 65 |
| 41446_f_at | 17.6 | 38.9 | 44.8 | 3.3E−06 | 66 |
| 41436_at | 15.8 | 9.71 | 7.05 | 3.3E−06 | 67 |
| 36672_at | 7 | 17.2 | 26 | 3.4E−06 | 68 |
| 38717_at | 6.62 | 15.1 | 21.0 | 3.7E−06 | 70 |
| 32183_at | 36.3 | 22.6 | 14.9 | 3.8E−06 | 71 |
| 32737_at | 14.2 | 26.4 | 36.9 | 4.3E−06 | 72 |
| 40140_at | 24.5 | 16.9 | 11.9 | 4.6E−06 | 73 |
| 40818_at | 23.9 | 14.1 | 10 | 4.7E−06 | 74 |
| 34787_at | 15.7 | 24.8 | 37.2 | 4.9E−06 | 75 |
| 38652_at | 7.05 | 13.5 | 15.5 | 5.0E−06 | 76 |
| 41812_s_at | 5.76 | 10.5 | 12.7 | 5.1E−06 | 77 |
| 31622_f_at | 18.5 | 34.9 | 38.3 | 6.3E−06 | 78 |
| 40828_at | 30.7 | 24.3 | 14.9 | 6.7E−06 | 79 |
| 39693_at | 3.76 | 9.10 | 11.0 | 7.7E−06 | 80 |
| 40994_at | 12.2 | 8.67 | 5.29 | 7.8E−06 | 81 |
| 39802_at | 24 | 13.6 | 5.19 | 9.1E−06 | 83 |
| 40567_at | 39.1 | 65.4 | 96.8 | 1.0E−05 | 84 |
| 38518_at | 16.7 | 11.9 | 6.67 | 1.1E−05 | 85 |
| 1368_at | 17.9 | 11.3 | 4 | 1.1E−05 | 87 |
| 41653_at | 8.29 | 4.57 | 3.29 | 1.3E−05 | 88 |
| 41669_at | 13.2 | 8.43 | 5.10 | 1.3E−05 | 89 |
| 36488_at | 7.81 | 17.7 | 25.5 | 1.4E−05 | 90 |
| 1005_at | 61.6 | 129 | 166 | 1.5E−05 | 91 |
| 36495_at | 71.2 | 37.3 | 26.4 | 1.7E−05 | 92 |
| 40579_at | 7.67 | 4.24 | 3.43 | 1.7E−05 | 93 |
| 36607_at | 6.05 | 11.9 | 16.2 | 2.0E−05 | 94 |
| 34311_at | 15.8 | 24.8 | 34.9 | 2.1E−05 | 95 |
| 35785_at | 92.4 | 71.7 | 45.3 | 2.1E−05 | 96 |
| 40622_r_at | 45 | 24.4 | 20.9 | 2.2E−05 | 97 |
| 33777_at | 7.43 | 20.9 | 31.7 | 2.2E−05 | 98 |
| 38220_at | 5.71 | 11.4 | 16.8 | 2.5E−05 | 99 |
| 1665_s_at | 6.86 | 26.5 | 39.9 | 2.6E−05 | 100 |
| 1693_s_at | 321 | 244 | 91.2 | 2.9E−05 | 101 |
| 32612_at | 5.90 | 8.86 | 12.1 | 2.9E−05 | 102 |
| 41045_at | 16.7 | 33.4 | 48.8 | 3.0E−05 | 103 |
| 37233_at | 19.9 | 9.52 | 2.86 | 3.0E−05 | 104 |
| 37967_at | 61.3 | 132 | 159 | 3.1E−05 | 105 |
| 2049_s_at | 11.9 | 26.9 | 32.8 | 3.3E−05 | 106 |
| 39997_at | 24.3 | 38.5 | 49.0 | 3.4E−05 | 107 |
| 41332_at | 8.14 | 13.7 | 18.1 | 3.4E−05 | 108 |
| 115_at | 51.5 | 35.0 | 9.19 | 3.6E−05 | 109 |
| 40964_at | 7.62 | 3.29 | 3.10 | 3.7E−05 | 110 |
| 35303_at | 25.8 | 19.6 | 12.0 | 4.0E−05 | 111 |
| 40742_at | 10 | 28.8 | 37.4 | 4.1E−05 | 112 |
| 37955_at | 4.14 | 5.81 | 8.38 | 4.4E−05 | 113 |
| 39698_at | 4.10 | 6.52 | 9.29 | 4.5E−05 | 114 |
| 32586_at | 6.05 | 9.67 | 12.4 | 4.5E−05 | 115 |
| 40159_r_at | 10.5 | 20.9 | 46.6 | 5.0E−05 | 116 |
| 32635_at | 8.24 | 3.95 | 3.57 | 5.0E−05 | 117 |
| 35840_at | 5.05 | 7.86 | 11.8 | 5.1E−05 | 118 |
| 37661_at | 21.2 | 13.3 | 6.48 | 5.1E−05 | 119 |
| 411_i_at | 29.2 | 40.1 | 65.6 | 5.1E−05 | 120 |
| 35012_at | 20.4 | 40.3 | 74.9 | 5.2E−05 | 121 |
| 38760_f_at | 9.33 | 16.0 | 21.4 | 5.3E−05 | 122 |
| 36474_at | 9.48 | 5.81 | 4.48 | 5.3E−05 | 123 |
| 41440_at | 4.24 | 9.38 | 9.10 | 5.4E−05 | 124 |
| 39953_i_at | 23.1 | 9.14 | 7.71 | 5.8E−05 | 125 |
| 245_at | 45.8 | 62.2 | 103 | 6.0E−05 | 126 |
| 37181_at | 3.67 | 5.90 | 7.52 | 6.0E−05 | 127 |
| 39799_at | 31.3 | 21.3 | 7.90 | 6.2E−05 | 128 |
| 39320_at | 6.48 | 12.4 | 16.1 | 6.4E−05 | 129 |
| 794_at | 5.29 | 10.2 | 14.0 | 6.4E−05 | 130 |
| 36091_at | 7.33 | 11.7 | 18.7 | 6.6E−05 | 131 |
| 37187_at | 66.8 | 45.1 | 11.7 | 7.2E−05 | 133 |
| 38338_at | 8.76 | 14.2 | 18.3 | 7.2E−05 | 134 |
| 34760_at | 14.3 | 23.0 | 31.0 | 7.3E−05 | 135 |
| 33133_at | 7.67 | 12.3 | 16.3 | 7.6E−05 | 136 |
| 32904_at | 10.2 | 19.4 | 26.2 | 7.7E−05 | 137 |
| 37359_at | 7.71 | 12.4 | 16.3 | 8.2E−05 | 138 |
| 40485_at | 3.95 | 7.81 | 9.81 | 8.3E−05 | 139 |
| 37215_at | 6.86 | 11.3 | 19.4 | 8.4E−05 | 140 |
| 40274_at | 9.10 | 21 | 16.1 | 8.5E−05 | 141 |
| 41174_at | 12.9 | 9.95 | 5.71 | 8.8E−05 | 143 |
| 1715_at | 5.86 | 14.0 | 21.5 | 8.8E−05 | 144 |
| 41503_at | 10.1 | 7.71 | 4.90 | 8.9E−05 | 145 |
| 40390_at | 5.71 | 3.62 | 2.81 | 9.2E−05 | 146 |
| 40811_at | 13.8 | 10.5 | 6.81 | 9.3E−05 | 147 |
| 31499_s_at | 11.7 | 24.6 | 41.3 | 9.3E−05 | 148 |
| 34023_at | 4.76 | 7.33 | 11.9 | 9.5E−05 | 149 |
| 1125_s_at | 8.57 | 6.29 | 4.24 | 9.7E−05 | 150 |
| 36617_at | 15.4 | 34.2 | 43.7 | 9.9E−05 | 151 |
| 36337_at | 15.2 | 7.48 | 5.95 | 0.00010 | 152 |
| 36161_at | 5.24 | 7.95 | 10.8 | 0.00010 | 153 |
| 1433_g_at | 22.0 | 15 | 8.62 | 0.00010 | 154 |
| 39043_at | 64.8 | 96.1 | 134 | 0.00011 | 155 |
| 37311_at | 67.7 | 94.7 | 139 | 0.00012 | 156 |
| 37009_at | 16.3 | 25.9 | 41.7 | 0.00013 | 157 |
| 33942_s_at | 17.9 | 9.52 | 7.86 | 0.00013 | 158 |
| 39641_at | 3.67 | 9.86 | 10.9 | 0.00013 | 159 |
| 529_at | 10.8 | 23.1 | 22.5 | 0.00013 | 160 |
| 32199_at | 8.81 | 2.90 | 2.95 | 0.00014 | 161 |
| 1426_at | 59.8 | 45.1 | 27.5 | 0.00014 | 162 |
| 31851_at | 17 | 12.5 | 8.10 | 0.00014 | 163 |
| 34378_at | 124 | 49.4 | 26.3 | 0.00014 | 164 |
| 36173_r_at | 6.90 | 2.52 | 3.81 | 0.00016 | 165 |
| 35275_at | 5 | 2.29 | 2.81 | 0.00017 | 166 |
| 41184_s_at | 7.33 | 14.8 | 18.0 | 0.00018 | 168 |
| 38391_at | 16.5 | 23.0 | 33.3 | 0.00018 | 170 |
| 39829_at | 28.0 | 17.8 | 13.6 | 0.00019 | 171 |
| 37587_at | 6.71 | 3.19 | 2.90 | 0.00020 | 172 |
| 34022_at | 35.0 | 20.0 | 3.52 | 0.00020 | 173 |
| 34188_at | 13.7 | 9.62 | 6.05 | 0.00022 | 175 |
| 37647_at | 13.2 | 25.3 | 35.2 | 0.00022 | 176 |
| 40089_at | 20.4 | 12.0 | 9.24 | 0.00023 | 177 |
| 39982_r_at | 7.52 | 4.76 | 2.95 | 0.00024 | 178 |
| 32202_at | 3.14 | 5.57 | 8.48 | 0.00026 | 179 |
| 35036_at | 7.33 | 2.71 | 2.81 | 0.00027 | 180 |
| 906_at | 34.6 | 23.6 | 12.6 | 0.00027 | 181 |
| 36033_at | 12.8 | 8.38 | 6.10 | 0.00028 | 182 |
| 36766_at | 20.8 | 36.2 | 57.2 | 0.00029 | 183 |
| 38893_at | 38.2 | 48.4 | 78.4 | 0.00029 | 184 |
| 37374_at | 3.86 | 6.38 | 8 | 0.00030 | 185 |
| 36599_at | 4.57 | 6.76 | 9.38 | 0.00030 | 186 |
| 464_s_at | 7.43 | 12.7 | 16.5 | 0.00030 | 187 |
| 32533_s_at | 6.05 | 13.1 | 17.0 | 0.00031 | 188 |
| 36496_at | 7.62 | 11 | 15.9 | 0.00031 | 189 |
| 36280_at | 7.62 | 13 | 18.4 | 0.00033 | 190 |
| 38126_at | 16.2 | 8.86 | 7.71 | 0.00033 | 191 |
| 37011_at | 22.4 | 38.9 | 52.2 | 0.00035 | 192 |
| 39166_s_at | 9.24 | 2.10 | 2.38 | 0.00037 | 193 |
| 1825_at | 16.6 | 28.0 | 35.2 | 0.00040 | 195 |
| 41577_at | 13.5 | 13.7 | 4.38 | 0.00041 | 196 |
| 33161_at | 3.14 | 6.10 | 9.81 | 0.00042 | 197 |
| 31438_s_at | 11.5 | 14.7 | 23.4 | 0.00043 | 198 |
| 36372_at | 5.05 | 12.6 | 19.7 | 0.00043 | 199 |
| 2094_s_at | 50.3 | 104 | 141 | 0.00045 | 200 |
| 32977_at | 11.6 | 22.6 | 33.4 | 0.00048 | 201 |
| 32264_at | 9.29 | 18.1 | 20.2 | 0.00048 | 202 |
| 32184_at | 9.90 | 16.4 | 20.0 | 0.00048 | 203 |
| 35674_at | 4.52 | 7.67 | 12 | 0.00049 | 204 |
| 41249_at | 5.43 | 8.90 | 14.3 | 0.00051 | 205 |
| 36762_at | 8.33 | 3.95 | 4.43 | 0.00052 | 206 |
| 37146_at | 13.3 | 5.24 | 4.90 | 0.00055 | 207 |
| 867_s_at | 13.7 | 7.24 | 3.57 | 0.00055 | 208 |
| 33500_i_at | 53.9 | 32.3 | 26.4 | 0.00056 | 209 |
| 1575_at | 7.71 | 5.67 | 3.57 | 0.00056 | 210 |
| 40088_at | 22.4 | 18.7 | 10.2 | 0.00060 | 211 |
| 37742_at | 6.19 | 9.81 | 14.5 | 0.00067 | 213 |
| 35992_at | 13.1 | 9.33 | 5.19 | 0.00072 | 214 |
| 35710_s_at | 6.29 | 10.6 | 13.0 | 0.00073 | 216 |
| 39119_s_at | 12.2 | 18.1 | 31.3 | 0.00074 | 217 |
| 34857_at | 14.9 | 9.43 | 6.29 | 0.00078 | 218 |
| 41198_at | 26 | 46.0 | 67.8 | 0.00080 | 219 |
| 34476_r_at | 11.5 | 8.48 | 3.81 | 0.00084 | 220 |
| 35772_at | 5.62 | 2.71 | 2.95 | 0.00086 | 221 |
| 34660_at | 13.1 | 20.3 | 28.4 | 0.00090 | 222 |
| 35648_at | 14.4 | 11.1 | 5.71 | 0.00097 | 223 |
| AFFX- | 65.8 | 30.3 | 34.9 | 0.00099 | 225 |
| HUMRGE/ | | | | | |
| M10098_3_at | | | | | |
| 31324_at | 7.43 | 3.71 | 3.48 | 0.00099 | 226 |
| 1395_at | 7.19 | 12.7 | 14.7 | 0.0010 | 227 |
| 32066_g_at | 27.2 | 22.4 | 12.2 | 0.0010 | 228 |
| 1107_s_at | 20.0 | 39.8 | 60 | 0.0010 | 229 |
| 1097_s_at | 29.0 | 21.7 | 9.86 | 0.0011 | 230 |
| 35591_at | 4.48 | 10 | 7.86 | 0.0011 | 231 |
| 32815_at | 17.2 | 7.86 | 6.33 | 0.0011 | 232 |
| 36231_at | 20.6 | 15.7 | 9.29 | 0.0011 | 233 |
| 38823_s_at | 10.8 | 9.76 | 5.10 | 0.0012 | 234 |
| 32618_at | 6.05 | 9.43 | 12.3 | 0.0013 | 235 |
| 2092_s_at | 9.57 | 6.62 | 2.67 | 0.0014 | 236 |
| 37184_at | 8.62 | 2.67 | 2.81 | 0.0014 | 237 |
| 37120_at | 15.8 | 8.71 | 3.48 | 0.0014 | 238 |
| 1237_at | 79.6 | 66.5 | 32.8 | 0.0015 | 239 |
| 37310_at | 9.76 | 9.38 | 3.95 | 0.0016 | 241 |
| 40541_at | 13.0 | 6.10 | 8.90 | 0.0016 | 242 |
| 31346_at | 4.90 | 10.5 | 7.10 | 0.0017 | 244 |
| 37875_at | 11.4 | 5.52 | 5.67 | 0.0017 | 245 |
| 38125_at | 8.24 | 3.29 | 3.19 | 0.0018 | 246 |
| 859_at | 22.0 | 19.3 | 6.86 | 0.0019 | 247 |
| 35792_at | 8.05 | 5.57 | 3.57 | 0.0020 | 249 |
| 34946_at | 5.48 | 8.14 | 11.2 | 0.0020 | 250 |
| 33956_at | 11.6 | 16.5 | 24.7 | 0.0023 | 253 |
| 32975_g_at | 8.86 | 4.90 | 4.29 | 0.0024 | 254 |
| 38582_at | 6.95 | 2.90 | 3.24 | 0.0025 | 255 |
| 181_g_at | 12 | 19.2 | 29.4 | 0.0025 | 256 |
| 37864_s_at | 10.0 | 6.29 | 3.43 | 0.0028 | 257 |
| 34704_r_at | 8.71 | 4.86 | 4.24 | 0.0028 | 258 |
| 32163_f_at | 10.2 | 6.10 | 4.76 | 0.0029 | 259 |
| 41189_at | 14 | 10 | 6.67 | 0.0030 | 260 |
| 41698_at | 6.81 | 6.24 | 3.33 | 0.0030 | 261 |
| 40456_at | 11.52 | 10.2 | 5.62 | 0.0031 | 262 |
| 41227_at | 15.8 | 7.52 | 10.8 | 0.0033 | 263 |
| 36100_at | 24.4 | 14.8 | 12.0 | 0.0035 | 264 |
| 36411_s_at | 16.2 | 8.19 | 7.90 | 0.0037 | 265 |
| 41167_at | 10.7 | 6.33 | 5.33 | 0.0037 | 266 |
| 41819_at | 4.48 | 6.71 | 9.29 | 0.0037 | 267 |
| 38389_at | 3.76 | 6.95 | 10.2 | 0.0038 | 268 |
| 37975_at | 14.9 | 35.2 | 41.1 | 0.0039 | 269 |
| 39640_at | 14.7 | 6.10 | 7.95 | 0.0043 | 270 |
| 38010_at | 7.38 | 3.90 | 3.33 | 0.0043 | 271 |
| 32675_at | 9.33 | 13.0 | 20.0 | 0.0044 | 272 |
| 1531_at | 8.52 | 4.52 | 3.90 | 0.0044 | 273 |
| 40646_at | 6.81 | 12.5 | 19.4 | 0.0045 | 274 |
| 39474_s_at | 4.71 | 2.33 | 3.05 | 0.0047 | 275 |
| 34498_at | 21 | 28.0 | 48.5 | 0.0053 | 276 |
| 37654_at | 4.05 | 6.95 | 8.14 | 0.0053 | 277 |
| 408_at | 24.4 | 18.2 | 3.48 | 0.0054 | 278 |
| 38340_at | 16.7 | 12.9 | 8.10 | 0.0056 | 279 |
| 1937_at | 31.6 | 13.0 | 9.62 | 0.0059 | 280 |
| 41638_at | 13.3 | 8.38 | 6.10 | 0.0059 | 281 |
| 36138_at | 21 | 32.5 | 42.5 | 0.0060 | 282 |
| 32407_f_at | 13.7 | 6.33 | 5.38 | 0.0061 | 283 |
| 38131_at | 17 | 36.1 | 2.90 | 0.0063 | 284 |
| 39775_at | 11.7 | 22.7 | 32.8 | 0.0064 | 285 |
| 318_at | 5.19 | 6.19 | 10.6 | 0.0073 | 286 |
| 39402_at | 83.1 | 87.2 | 12.4 | 0.0077 | 287 |
| 36543_at | 7.57 | 5.62 | 2.67 | 0.0079 | 288 |
| 1788_s_at | 7.33 | 6.62 | 3.62 | 0.0091 | 289 |
| 34702_f_at | 10.6 | 4.67 | 3.86 | 0.0094 | 290 |
| 40951_at | 5.71 | 2.76 | 2.86 | 0.0098 | 291 |
| 35313_at | 5.43 | 3 | 2.38 | 0.010 | 292 |
| 1520_s_at | 105 | 112 | 16 | 0.011 | 293 |
| 40171_at | 6.05 | 8.14 | 12.8 | 0.011 | 295 |
| 32162_r_at | 14.1 | 7.86 | 6 | 0.012 | 296 |
| 1369_s_at | 365 | 292 | 163 | 0.012 | 297 |
| 34490_f_at | 5.05 | 7.71 | 10.2 | 0.012 | 298 |
| 36710_at | 8.81 | 13.5 | 22.2 | 0.013 | 300 |
| 37220_at | 8.24 | 14.0 | 20.0 | 0.013 | 301 |
| 32003_at | 9.05 | 5.29 | 4.38 | 0.013 | 302 |
| 1667_s_at | 14.4 | 9.57 | 7.14 | 0.013 | 303 |
| 731_f_at | 7.71 | 4.05 | 3.76 | 0.015 | 304 |
| 39448_r_at | 8.38 | 4 | 4.43 | 0.015 | 305 |
| 40385_at | 7 | 11.0 | 2.67 | 0.017 | 306 |
| 37603_at | 83.9 | 88.7 | 36.3 | 0.028 | 307 |
| 41046_s_at | 5.52 | 2.57 | 3.19 | 0.040 | 308 |
| 37456_at | 16 | 30.0 | 37.6 | 0.039 | 309 |
| 31828_r_at | 9.52 | 2.52 | 6 | 0.047 | 310 |

Each qualifier has a corresponsding CCI-779 activity gene probe sequence (CPS), and each CPS can be derived from a corresponding SEQ ID NO depicted in Table 3. In many cases, each CPS consists of an unambiguous fragment, or the complement thereof, of the corresponding SEQ ID NO. In many other cases, each CPS also comprises at least one oligonucleotide probe of the corresponding qualifier. Each SEQ ID NO in Table 3 is a cDNA or genomic sequence, or the complement thereof, of a CCI-779 activity gene represented by the qualifier that corresponds to the SEQ ID NO. Accordingly, each SEQ ID NO or its corresponding CPS can hybridize under stringent or nucleic acid array hybridization conditions to the RNA transcripts, or the complements thereof, of the represented CCI-779 activity gene.

Each SEQ ID NO may have an Entrez nucleotide sequence database accession number (see Table 4). The Entrez nucleotide sequence database is maintained by the National Center of Biotechnology Information (NCBI), National Library of Medicine, Washington, D.C. The database collects sequences from several sources, including GenBank, RefSeq, and PDB.

Any ambiguous residue (“n”) in a SEQ ID NO can be determined by numerous methods. In one embodiment, the ambiguous residues in a SEQ ID NO are determined by aligning the SEQ ID NO to a corresponding genomic sequence obtained from a human genome sequence database. In another embodiment, the ambiguous residues in a SEQ ID NO are determined based on the sequence of the corresponding Entrez accession number. In yet another embodiment, the ambiguous residues are determined by re-sequencing the SEQ ID NO. In general, each “n” position in a SEQ ID NO represents at least one nucleotide selected from a, t, g, and c, or contains no nucleotide residue.

TABLE 3
Qualifiers and Corresponding CPSs and SEQ ID NOs

| | | |
|---|---|---|
| Qualifier | CPS | SEQ ID NO |

| | | |
|---|---|---|
| 34768_at | 1 | nucleotides 1777 to 2353 of SEQ ID NO: 1 |
| 36097_at | 2 | nucleotides 1273 to 1774 of SEQ ID NO: 2 |
| 36675_r_at | 3 | nucleotides 338 to 744 of SEQ ID NO: 3 |
| 37940_f_at | 4 | the complement of nucleotides 305 to 502 of SEQ ID |
| | | NO:4 |
| 34338_at | 5 | nucleotides 659 to 995 of SEQ ID NO: 5 |
| 38991_at | 6 | the complement of nucleotides 382 to 527 of SEQ ID |
| | | NO:6 |
| 38976_at | 7 | nucleotides 1107 to 1517 of SEQ ID NO: 7 |
| 37905_r_at | 8 | nucleotides 1451 to 1961 of SEQ ID NO: 8 |
| 38780_at | 9 | nucleotides 567 to 1095 of SEQ ID NO: 9 |
| 41215_s_at | 10 | nucleotides 719 to 1049 of SEQ ID NO: 10 |
| 35956_s_at | 11 | nucleotides 1360 to 1973 of SEQ ID NO: 11 |
| 32332_at | 13 | nucleotides 1190 to 1720 of SEQ ID NO: 12 |
| 41551_at | 14 | the complement of nucleotides 223 to 388 of SEQ ID |
| | | NO: 13 |
| 39332_at | 15 | nucleotides 1091 to 1182 of SEQ ID NO: 14 |
| 40778_at | 16 | nucleotides 498 to 938 of SEQ ID NO: 15 |
| 32232_at | 17 | nucleotides 484 to 963 of SEQ ID NO: 16 |
| 34310_at | 18 | SEQ ID NO: 17 |
| 1184_at | 19 | nucleotides 197 to 743 of SEQ ID NO: 18 |
| 35688_g_at | 20 | SEQ ID NO: 19 |
| 38981_at | 21 | SEQ ID NO: 20 |
| 37391_at | 23 | nucleotides 1022-1395 of SEQ ID NO: 21 |
| 39593_at | 24 | the complement of nucleotides 14 to 199 of SEQ ID NO: |
| | | 22 |
| 32505_at | 25 | nucleotides 4 to 223 of SEQ ID NO: 23 |
| AFFX- | 26 | SEQ ID NO: 24 |
| M27830_5_at | | |
| 35666_at | 27 | nucleotides 3050 to 3530 of SEQ ID NO: 25 |
| 39061_at | 28 | nucleotides 425 to 948 of SEQ ID NO: 26 |
| 39748_at | 29 | nucleotides 2675 to 3052 of SEQ ID NO: 27 |
| 40505_at | 30 | the complement of nucleotides 52 to 286 of SEQ ID NO: |
| | | 28 |
| 38597_f_at | 31 | nucleotides 2196 to 2257 of SEQ ID NO: 29 |
| 39545_at | 32 | SEQ ID NO: 30 |
| 34268_at | 33 | nucleotides 1088 to 1530 of SEQ ID NO: 31 |
| 38816_at | 34 | nucleotides 3244 to 3649 of SEQ ID NO: 32 |
| 38732_at | 35 | nucleotides 762-1229 of SEQ ID NO: 33 |
| 40607_at | 36 | nucleotides 5022-5382 of SEQ ID NO: 34 |
| 37200_at | 37 | nucleotides 1406-1932 of SEQ ID NO: 35 |
| 283_at | 38 | nucleotides 1426_1965 of SEQ ID NO: 36 |
| 40757_at | 39 | nucleotides 393-818 of SEQ ID NO: 37 |
| 384_at | 41 | SEQ ID NO: 38 |
| 40814_at | 42 | nucleotides 1123-1247 of SEQ ID NO: 39 |
| 37027_at | 43 | SEQ ID NO: 40 |
| 1760_s_at | 44 | nucleotides 2049-2393 of SEQ ID NO: 41 |
| 1292_at | 45 | nucleotides 1126-1654 of SEQ ID NO: 42 |
| 38287_at | 46 | the complement of nucleotides 8-451 of SEQ ID NO: 43 |
| 41591_at | 47 | the complement of nucleotides 39-547 of SEQ ID NO: |
| | | 44 |
| 32475_at | 48 | nucleotides 1845-2329 of SEQ ID NO: 45 |
| 40782_at | 49 | nucleotides 886-1289 of SEQ ID NO: 46 |
| 503_at | 50 | nucleotides 13-342 of SEQ ID NO: 47 |
| 875_g_at | 51 | nucleotides 562-886 of SEQ ID NO: 48 |
| 37185_at | 52 | nucleotides 1311-1761 of SEQ ID NO: 49 |
| 32282_at | 53 | nucleotides 2851-3265 of SEQ ID NO: 50 |
| 40981_at | 54 | nucleotides 2661-3257 of SEQ ID NO: 51 |
| 34782_at | 55 | the complement of nucleotides 529-952 of SEQ ID NO: |
| | | 52 |
| 37373_at | 56 | nucleotides 1339-1771 of SEQ ID NO: 53 |
| 40018_at | 57 | nucleotides 5780 to 6213 of SEQ ID NO: 54 |
| 31638_at | 58 | SEQ ID NO: 55 |
| 38191_at | 59 | the complement of nucleotides 171-451 of SEQ ID NO: |
| | | 56 |
| 33396_at | 60 | SEQ ID NO: 57 |
| 38104_at | 61 | SEQ ID NO: 58 |
| 32193_at | 62 | SEQ ID NO: 59 |
| 38350_f_at | 63 | SEQ ID NO: 60 |
| 1985_s_at | 64 | nucleotides 90-510 of SEQ ID NO: 61 |
| 38598_at | 65 | the complement of nucleotides 149-213 of SEQ ID NO: |
| | | 62 |
| 41446_f_at | 66 | the complement of nucleotides 28-303 of SEQ ID NO: |
| | | 63 |
| 41436_at | 67 | nucleotides 4687-4907 of SEQ ID NO: 64 |
| 36672_at | 68 | nucleotides 1509-1977 of SEQ ID NO: 65 |
| 38717_at | 70 | nucleotides 1355 to 1737 of SEQ ID NO: 66 |
| 32183_at | 71 | nucleotides 2195 to 2651 of SEQ ID NO: 67 |
| 32737_at | 72 | nucleotides 183 to 669 of SEQ ID NO: 68 |
| 40140_at | 73 | nucleotides 2828 to 3356 of SEQ ID NO: 69 |
| 40818_at | 74 | nucleotides 5138-5360 of SEQ ID NO: 70 |
| 34787_at | 75 | nucleotides 3428-3616 of SEQ ID NO: 71 |
| 38652_at | 76 | nucleotides 829-1333 of SEQ ID NO: 72 |
| 41812 s_at | 77 | nucleotides 3618-4192 of SEQ ID NO: 73 |
| 31622_f_at | 78 | SEQ ID NO: 74 |
| 40828_at | 79 | nucleotides 4472-4977 of SEQ ID NO: 75 |
| 39693_at | 80 | the complement of nucleotides 55-554 of SEQ ID NO: |
| | | 76 |
| 40994_at | 81 | nucleotides 2004 to 2502 of SEQ ID NO: 77 |
| 39802_at | 83 | nucleotides 444 to 991 of SEQ ID NO: 78 |
| 40567_at | 84 | nucleotides 3868 to 3916 of SEQ ID NO: 79 |
| 38518_at | 85 | nucleotides 3841-4076 of SEQ ID NO: 80 |
| 1368_at | 87 | nucleotides 4459-4885 of SEQ ID NO: 81 |
| 41653_at | 88 | nucleotides 110125 to 110701 of SEQ ID NO: 82 |
| 41669_at | 89 | nucleotides 4596 to 5049 of SEQ ID NO: 83 |
| 36488_at | 90 | nucleotides 4959 to 5449 of SEQ ID NO: 84 |
| 1005_at | 91 | nucleotides 784 to 1318 of SEQ ID NO: 85 |
| 36495_at | 92 | SEQ ID NO: 86 |
| 40579_at | 93 | SEQ ID NO: 87 |
| 36607_at | 94 | the complement of 201545 to 202131 of SEQ ID NO: 88 |
| 34311_at | 95 | nucleotides 360 to 722 of SEQ ID NO: 89 |
| 35785_at | 96 | nucleotides 160 to 436 of SEQ ID NO: 90 |
| 40622_r_at | 97 | SEQ ID NO: 91 |
| 33777_at | 98 | SEQ ID NO: 92 |
| 38220_at | 99 | nucleotides 3894 to 4357 of SEQ ID NO: 93 |
| 1665_s_at | 100 | SEQ ID NO: 94 |
| 1693_s_at | 101 | SEQ ID NO: 95 |
| 32612_at | 102 | nucleotides 2033 to 2405 of SEQ ID NO: 96 |
| 41045_at | 103 | nucleotides 1546 to 1973 of SEQ ID NO: 97 |
| 37233_at | 104 | SEQ ID NO: 98 |
| 37967_at | 105 | nucleotides 278 to 635 of SEQ ID NO: 99 |
| 2049_s_at | 106 | nucleotides 842 to 1443 of SEQ ID NO: 100 |
| 39997_at | 107 | SEQ ID NO: 101 |
| 41332_at | 108 | nucleotides 635 to 1169 of SEQ ID NO: 102 |
| 115_at | 109 | nucleotides 3083-3605 of SEQ ID NO: 103 |
| 40964_at | 110 | SEQ ID NO: 104 |
| 35303_at | 111 | SEQ ID NO: 105 |
| 40742_at | 112 | nucleotides 1661 to 1912 of SEQ ID NO: 106 |
| 37955_at | 113 | nucleotides 669 to 730 of SEQ ID NO: 107 |
| 39698_at | 114 | nucleotides 599 to 1068 of SEQ ID NO: 108 |
| 32586_at | 115 | nucleotides 5059 to 5585 of SEQ ID NO: 109 |
| 40159_r_at | 116 | nucleotides 970-1341 of SEQ ID NO: 110 |
| 32635_at | 117 | nucleotides 3240 to 3424 of SEQ ID NO: 111 |
| 35840_at | 118 | nucleotides 639 to 1163 of SEQ ID NO: 112 |
| 37661_at | 119 | nucleotides 4061-4398 of SEQ ID NO: 113 |
| 411_i_at | 120 | nucleotides 281 to 318 of SEQ ID NO: 114 |
| 35012_at | 121 | nucleotides 1070 to 1667 of SEQ ID NO: 115 |
| 38760_f_at | 122 | nucleotides 1014 to 1263 of SEQ ID NO: 116 |
| 36474_at | 123 | nucleotides 3881 to 4038 of SEQ ID NO: 117 |
| 41440_at | 124 | nucleotides 655 to 933 of SEQ ID NO: 118 |
| 39953_i_at | 125 | nucleotides 5549 to 5598 of SEQ ID NO: 119 |
| 245_at | 126 | nucleotides 1795 to 2323 of SEQ ID NO: 120 |
| 37181_at | 127 | nucleotides 375 to 947 of SEQ ID NO: 121 |
| 39799_at | 128 | SEQ ID NO: 122 |
| 39320_at | 129 | nucleotides 569 to 1121 of SEQ ID NO: 123 |
| 794_at | 130 | nucleotides 1484 to 2024 of SEQ ID NO: 124 |
| 36091_at | 131 | nucleotides 1806 to 1973 of SEQ ID NO: 125 |
| 37187_at | 133 | nucleotides 504-946 of SEQ ID NO: 126 |
| 38338_at | 134 | the complement of nucleotides 61 to 470 of SEQ ID NO: |
| | | 127 |
| 34760_at | 135 | nucleotides 3404 to 3694 of SEQ ID NO: 128 |
| 33133_at | 136 | SEQ ID NO: 129 |
| 32904_at | 137 | SEQ ID NO: 130 |
| 37359_at | 138 | nucleotides 798 to 1300 of SEQ ID NO: 131 |
| 40485_at | 139 | the complement of nucleotides 35-188 of SEQ ID NO: |
| | | 132 |
| 37215_at | 140 | SEQ ID NO: 133 |
| 40274_at | 141 | nucleotides 561-736 of SEQ ID NO: 134 |
| 41174_at | 143 | nucleotides 3461 to 4011 of SEQ ID NO: 135 |
| 1715_at | 144 | nucleotides 1162 to 1402 of SEQ ID NO: 136 |
| 41503_at | 145 | nucleotides 3580 to 4066 of SEQ ID NO: 137 |
| 40390_at | 146 | nucleotides 978 to 1393 of SEQ ID NO: 138 |
| 40811_at | 147 | nucleotides 4475 to 4934 of SEQ ID NO: 139 |
| 31499_s_at | 148 | nucleotides 251-854 of SEQ ID NO: 140 |
| 34023_at | 149 | nucleotides 570 to 1026 of SEQ ID NO: 141 |
| 1125_s_at | 150 | SEQ ID NO: 142 |
| 36617_at | 151 | nucleotides 691 to 819 of SEQ ID NO: 143 |
| 36337_at | 152 | the complement of nucleotides 54-372 of SEQ ID NO: |
| | | 144 |
| 36161_at | 153 | nucleotides 5172 to 5682 of SEQ ID NO: 145 |
| 1433_g_at | 154 | nucleotides 1907 to 2267 of SEQ ID NO: 146 |
| 39043_at | 155 | nucleotides 967 to 1366 of SEQ ID NO: 147 |
| 37311_at | 156 | SEQ ID NO: 148 |
| 37009_at | 157 | the complement of nucleotides 3527-3999 of SEQ ID |
| | | NO: 149 |
| 33942_s_at | 158 | nucleotides 3457 to 3843 of SEQ ID NO: 150 |
| 39641_at | 159 | nucleotides 759 to 1194 of SEQ ID NO: 151 |
| 529_at | 160 | nucleotides 1995 to 2319 of SEQ ID NO: 152 |
| 32199_at | 161 | nucleotides 4778 to 5059 of SEQ ID NO: 153 |
| 1426_at | 162 | nucleotides 2046 to 2334 of SEQ ID NO: 154 |
| 31851_at | 163 | nucleotides 802 to 1201 of SEQ ID NO: 155 |
| 34378_at | 164 | nucleotides 1217-1314 of SEQ ID NO: 156 |
| 36173_r_at | 165 | nucleotides 4756 to 4950 of SEQ ID NO: 157 |
| 35275_at | 166 | nucleotides 3552 to 3973 of SEQ ID NO: 158 |
| 41184_s_at | 168 | SEQ ID NO: 159 |
| 38391_at | 170 | nucleotides 750 to 1184 of SEQ ID NO: 160 |
| 39829_at | 171 | nucleotides 799 to 1337 of SEQ ID NO: 161 |
| 37587_at | 172 | nucleotides 599 to 1167 of SEQ ID NO: 162 |
| 34022_at | 173 | nucleotides 426-993 of SEQ ID NO: 163 |
| 34188_at | 175 | nucleotides 1283 to 1708 of SEQ ID NO: 164 |
| 37647_at | 176 | nucleotides 1702 to 2244 of SEQ ID NO: 165 |
| 40089_at | 177 | nucleotides 1189 to 1234 of SEQ ID NO: 166 |
| 39982_r_at | 178 | nucleotides 1075 to 1489 of SEQ ID NO: 167 |
| 32202_at | 179 | nucleotides 1928 to 2496 of SEQ ID NO: 168 |
| 35036_at | 180 | nucleotides 2895 to 3261 of SEQ ID NO: 169 |
| 906_at | 181 | nucleotides 1993 to 2533 of SEQ ID NO: 170 |
| 36033_at | 182 | nucleotides 562 to 916 of SEQ ID NO: 171 |
| 36766_at | 183 | nucleotides 167-666 of SEQ ID NO: 172 |
| 38893_at | 184 | nucleotides 29281 to 29344 of SEQ ID NO: 173 |
| 37374_at | 185 | nucleotides 1465-1939 of SEQ ID NO: 174 |
| 36599_at | 186 | SEQ ID NO: 175 |
| 464_s_at | 187 | nucleotides 869-1048 of SEQ ID NO: 176 |
| 32533_s_at | 188 | nucleotides 91-464 of SEQ ID NO: 177 |
| 36496_at | 189 | nucleotides 1061-1514 of SEQ ID NO: 178 |
| 36280_at | 190 | nucleotides 535-953 of SEQ ID NO: 179 |
| 38126_at | 191 | SEQ ID NO: 180 |
| 37011_at | 192 | nucleotides 37 to 460 of SEQ ID NO: 181 |
| 39166_s_at | 193 | nucleotides 1583-1790 of SEQ ID NO: 182 |
| 1825_at | 195 | nucleotides 6960-7542 of SEQ ID NO: 183 |
| 41577_at | 196 | nucleotides 5804-6213 of SEQ ID NO: 184 |
| 33161_at | 197 | the complement of nucleotides 1 to 270 of SEQ ID NO: |
| | | 185 |
| 31438_s_at | 198 | nucleotides 3247-3720 of SEQ ID NO: 186 |
| 36372_at | 199 | nucleotides 2437-3029 of SEQ ID NO: 187 |
| 2094 s_at | 200 | nucleotides 2713-3294 of SEQ ID NO: 188 |
| 32977_at | 201 | nucleotides 2074-2206 of SEQ ID NO: 189 |
| 32264_at | 202 | nucleotides 493-755 of SEQ ID NO: 190 |
| 32184_at | 203 | nucleotides 1691-2175 of SEQ ID NO: 191 |
| 35674_at | 204 | nucleotides 3798-4194 of SEQ ID NO: 192 |
| 41249_at | 205 | the complement of nucleotides 63056-63481 of SEQ ID |
| | | NO: 193 |
| 36762_at | 206 | nucleotides 1129-1630 of SEQ ID NO: 194 |
| 37146_at | 207 | nucleotides 5897-6262 of SEQ ID NO: 195 |
| 867_s_at | 208 | nucleotides 1821-1945 of SEQ ID NO: 196 |
| 33500_i_at | 209 | nucleotides 1253-1296 of SEQ ID NO: 197 |
| 1575_at | 210 | nucleotides 4240-4585 of SEQ ID NO: 198 |
| 40088_at | 211 | nucleotides 6655-7207 of SEQ ID NO: 199 |
| 37742_at | 213 | nucleotides 1946-2278 of SEQ ID NO: 200 |
| 35992_at | 214 | nucleotides 1207-1652 of SEQ ID NO: 201 |
| 35710_s_at | 216 | nucleotides 63-630 of SEQ ID NO: 202 |
| 39119_s_at | 217 | nucleotides 408-727 of SEQ ID NO: 203 |
| 34857_at | 218 | nucleotides 1269-1779 of SEQ ID NO: 204 |
| 41198_at | 219 | nucleotides 1589-2106 of SEQ ID NO: 205 |
| 34476_r_at | 220 | nucleotides 4012-4358 of SEQ ID NO: 206 |
| 35772_at | 221 | SEQ ID NO: 207 |
| 34660_at | 222 | the complement of nucleotides 40-615 of SEQ ID NO: |
| | | 208 |
| 35648_at | 223 | SEQ ID NO: 209 |
| AFFX-HUMRGE/ | 225 | nucleotides 62-1969 of SEQ ID NO: 210 |
| M10098_3_at | | |
| 31324_at | 226 | nucleotides 51-308 of SEQ ID NO: 211 |
| 1395_at | 227 | nucleotides 433-1039 of SEQ ID NO: 212 |
| 32066_g_at | 228 | SEQ ID NO: 213 |
| 1107_s_at | 229 | nucleotides 7-602 of SEQ ID NO: 214 |
| 1097_s_at | 230 | SEQ ID NO: 215 |
| 35591_at | 231 | nucleotides 1711-2087 of SEQ ID NO: 216 |
| 32815_at | 232 | the complement of nucleotides 49-265 of SEQ ID NO: |
| | | 217 |
| 36231_at | 233 | SEQ ID NO: 218 |
| 38823_s_at | 234 | the complement of nucleotides 57-557 of SEQ ID NO: |
| | | 219 |
| 32618_at | 235 | nucleotides 489-909 of SEQ ID NO: 220 |
| 2092_s_at | 236 | nucleotides 824-1229 of SEQ ID NO: 221 |
| 37184_at | 237 | nucleotides 1631-2037 of SEQ ID NO: 222 |
| 37120_at | 238 | nucleotides 1870-2379 of SEQ ID NO: 223 |
| 1237_at | 239 | nucleotides 658-1204 of SEQ ID NO: 224 |
| 37310_at | 241 | nucleotides 6668-7083 of SEQ ID NO: 225 |
| 40541_at | 242 | nucleotides 1030-1412 of SEQ ID NO: 226 |
| 31346_at | 244 | nucleotides 647-1187 of SEQ ID NO: 227 |
| 37875_at | 245 | nucleotides 2330-2672 of SEQ ID NO: 228 |
| 38125_at | 246 | nucleotides 2357-2473 of SEQ ID NO: 229 |
| 859_at | 247 | nucleotides 4693-5071 of SEQ ID NO: 230 |
| 35792_at | 249 | nucleotides 884-1174 of SEQ ID NO: 231 |
| 34946_at | 250 | nucleotides 386-895 of SEQ ID NO: 232 |
| 33956_at | 253 | nucleotides 90-594 of SEQ ID NO: 233 |
| 32975_g_at | 254 | nucleotides 18770-18875 of SEQ ID NO: 234 |
| 38582_at | 255 | the complement of nucleotides 40 to 288 of SEQ ID NO: |
| | | 235 |
| 181_g_at | 256 | nucleotides 1464-1864 of SEQ ID NO: 236 |
| 37864_s_at | 257 | nucleotides 1108-1568 of SEQ ID NO: 237 |
| 34704_r_at | 258 | SEQ ID NO: 238 |
| 32163_f_at | 259 | SEQ ID NO: 239 |
| 41189_at | 260 | nucleotides 1161-1608 of SEQ ID NO: 240 |
| 41698_at | 261 | nucleotides 45240-45830 of SEQ ID NO: 241 |
| 40456_at | 262 | nucleotides 733-1310 of SEQ ID NO: 242 |
| 41227_at | 263 | nucleotides 35752-35804 of SEQ ID NO: 243 |
| 36100_at | 264 | nucleotides 2613-3118 of SEQ ID NO: 244 |
| 36411_s_at | 265 | nucleotides 1623-2080 of SEQ ID NO: 245 |
| 41167_at | 266 | nucleotides 1596-2100 of SEQ ID NO: 246 |
| 41819_at | 267 | nucleotides 2242-2578 of SEQ ID NO: 247 |
| 38389_at | 268 | nucleotides 960-1377 of SEQ ID NO: 248 |
| 37975_at | 269 | nucleotides 3684-4231 of SEQ ID NO: 249 |
| 39640_at | 270 | nucleotides 2415-2938 of SEQ ID NO: 250 |
| 38010_at | 271 | nucleotides 1044-1494 of SEQ ID NO: 251 |
| 32675_at | 272 | nucleotides 972-1411 of SEQ ID NO: 252 |
| 1531_at | 273 | nucleotides 1691-1891 of SEQ ID NO: 253 |
| 40646_at | 274 | nucleotides 2496-3012 of SEQ ID NO: 254 |
| 39474_s_at | 275 | nucleotides 22-507 of SEQ ID NO: 255 |
| 34498_at | 276 | nucleotides 1434-1983 of SEQ ID NO: 256 |
| 37654_at | 277 | nucleotides 1580-2043 of SEQ ID NO: 257 |
| 408_at | 278 | nucleotides 1412-1851 of SEQ ID NO: 258 |
| 38340_at | 279 | nucleotides 4103-4434 of SEQ ID NO: 259 |
| 1937_at | 280 | SEQ ID NO: 260 |
| 41638_at | 281 | nucleotides 1696-1922 of SEQ ID NO: 261 |
| 36138_at | 282 | nucleotides 943-1477 of SEQ ID NO: 262 |
| 32407_f_at | 283 | nucleotides 3-280 of SEQ ID NO: 263 |
| 38131_at | 284 | nucleotides 1240-1517 of SEQ ID NO: 264 |
| 39775_at | 285 | nucleotides 18180-18249 of SEQ ID NO: 265 |
| 318_at | 286 | nucleotides 601-838 of SEQ ID NO: 266 |
| 39402_at | 287 | nucleotides 927-1473 of SEQ ID NO: 267 |
| 36543_at | 288 | nucleotides 1723-2013 of SEQ ID NO: 268 |
| 1788_s_at | 289 | nucleotides 1622-2058 of SEQ ID NO: 269 |
| 34702_f_at | 290 | nucleotides 534-575 of SEQ ID NO: 270 |
| 40951_at | 291 | nucleotides 1860-2099 of SEQ ID NO: 271 |
| 35313_at | 292 | nucleotides 6412-6826 of SEQ ID NO: 272 |
| 1520_s_at | 293 | SEQ ID NO: 273 |
| 40171_at | 295 | SEQ ID NO: 274 |
| 32162_r_at | 296 | nucleotides 420-480 of SEQ ID NO: 275 |
| 1369_s_at | 297 | nucleotides 3645-4005 of SEQ ID NO: 276 |
| 34490_f_at | 298 | SEQ ID NO: 277 |
| 36710_at | 300 | nucleotides 74-557 of SEQ ID NO: 278 |
| 37220_at | 301 | SEQ ID NO: 279 |
| 32003_at | 302 | nucleotides 1144-1487 of SEQ ID NO: 280 |
| 1667_s_at | 303 | nucleotides 1531-1603 of SEQ ID NO: 281 |
| 731_f_at | 304 | SEQ ID NO: 282 |
| 39448_r_at | 305 | the complement of nucleotides 46-344 of SEQ ID NO: |
| | | 283 |
| 40385_at | 306 | nucleotides 207-742 of SEQ ID NO: 284 |
| 37603_at | 307 | nucleotides 1184-1653 of SEQ ID NO: 285 |
| 41046_s_at | 308 | nucleotides 5551-6046 of SEQ ID NO: 286 |
| 37456_at | 309 | SEQ ID NO: 287 |
| 31828_r_at | 310 | nucleotides 1750 to 2274 of SEQ ID NO: 288 |

TABLE 4
SEQ ID Nos and Corresponding Entrez Accession Nos

| | | |
|---|---|---|
| SEQ ID NO | Entrez Accession No. | Reported Source of Entrez Sequence |

| | | |
|---|---|---|
| 1 | AL080080 | Homo sapiens mRNA; cDNA |
| | | DKFZp564E1962 (from clone |
| | | DKFZp564E1962) |
| 2 | M62831 | Human transcription factor ETR101 |
| | | mRNA |
| 3 | J03191 | Human profilin mRNA |
| 4 | AA806768 | |
| 5 | D49738 | Human cytoskeleton associated protein |
| | | (CG22) mRNA |
| 6 | U55980 | |
| 7 | D44497 | Human mRNA for actin binding protein |
| | | p57 |
| 8 | X66436 | H.sapiens hsr1 mRNA |
| 9 | J04794 | Human aldehyde reductase mRNA |
| 10 | D13891 | Human mRNA for Id-2H |
| 11 | U18467 | Human pregnancy-specific beta 1- |
| | | glycoprotein 7 (PSG7) mRNA |
| 12 | X69433 | H.sapiens mRNA for mitochondrial |
| | | isocitrate dehydrogenase (NADP⁺) |
| 13 | AW044624 | |
| 14 | AF035316 | Homo sapiens clone 23678 mRNA |
| 15 | AF035555 | Homo sapiens 17beta-hydroxysteroid |
| | | dehydrogenase type 10/short chain L-3- |
| | | hydroxyacyl-CoA dehydrogenase |
| | | (HSD17B10/SCHAD) mRNA; nuclear |
| | | gene for mitochondrial product. |
| 16 | AF047181 | Homo sapiens NADH-ubiquinone |
| | | oxidoreductase subunit CI-SGDH mRNA |
| 18 | D45248 | Human mRNA for proteasome activator |
| | | hPA28 subunit beta |
| 21 | X12451 | Human mRNA for pro-cathepsin L (major |
| | | excreted protein MEP) |
| 22 | AI432401 | |
| 23 | W28652 | |
| 24 | M27830 | Human 28S ribosomal RNA gene |
| 25 | U38276 | Human semaphorin III family homolog |
| | | mRNA |
| 26 | D28137 | Human mRNA for BST-2 |
| 27 | AL050021 | Homo sapiens mRNA; cDNA |
| | | DKFZp564D016 (from clone |
| | | DKFZp564D016) |
| 28 | AA883502 | |
| 29 | D50402 | Human mRNA for NRAMP1 |
| 31 | X91809 | H.sapiens mRNA for GAIP protein. |
| 32 | AF095791 | Homo sapiens TACC2 protein (TACC2) |
| | | mRNA |
| 33 | X91788 | H.sapiens mRNA for Icln protein |
| 34 | U97105 | Homo sapiens N2A3 mRNA |
| 35 | J04162 | Human leukocyte IgG receptor (Fc- |
| | | gamma-R) mRNA |
| 36 | L16842 | Human ubiquinol cytochrome-c reductase |
| | | core I protein mRNA |
| 37 | M18737 | Human Hanukah factor serine protease |
| | | (HuHF) mRNA |
| 39 | L40586 | Homo sapiens iduronate-2-sulphatase |
| | | (IDS) mRNA |
| 41 | D11327 | Human mRNA for protein-tyrosine |
| | | phosphatase |
| 42 | L11329 | Homo sapiens protein tyrosine |
| | | phosphatase (PAC-1) mRNA |
| 43 | AA808961 | |
| 44 | AI652978 | |
| 45 | AF025529 | Homo sapiens leucocyte immunoglobulin- |
| | | like receptor-6b (LIR-6) mRNA |
| 46 | AF061741 | Homo sapiens retinal short-chain |
| | | dehydrogenase/reductase retSDR1 mRNA |
| 47 | U37690 | Human RNA polymerase II subunit |
| | | (hsRPB10) mRNA |
| 48 | M26683 | Human interferon gamma treatment |
| | | inducible mRNA |
| 49 | Y00630 | Human mRNA for Arg-Serpin |
| | | (plasminogen activator-inhibitor 2, PAI-2) |
| 50 | U66047 | Homo sapiens clone Z′3-1 placenta |
| | | expressed mRNA from chromosome X |
| 51 | U00930 | Human clone C4E 1.63 (CAC)n/(GTG)n |
| | | repeat-containing mRNA |
| 52 | AL021938 | Homo sapiens DNA sequence from PAC |
| | | 232K4 on chromosome 6p22.3. Contains |
| | | the JUMONJI gene for a hypothetical |
| | | 141.7 kD protein. Contains ESTs, STSs, a |
| | | CA repeat polymorphism and genomic |
| | | marker D6S260′ |
| 53 | U27460 | Human uridine diphosphoglucose |
| | | pyrophosphorylase mRNA |
| 54 | AB007870 | Homo sapiens KIAA0410 mRNA |
| 56 | AI040181 | |
| 61 | X73066 | H.sapiens NM23-H1 mRNA |
| 62 | AI679353 | |
| 64 | AJ224901 | Homo sapiens mRNA for ZNF198 protein. |
| 65 | L13977 | Human prolylcarboxypeptidase mRNA |
| 66 | AL050159 | Homo sapiens mRNA; cDNA |
| | | DKFZp586A0522 (from clone |
| | | DKFZp586A0522) |
| 67 | M74002 | Human arginine-rich nuclear protein |
| | | mRNA |
| 68 | M64595 | Human small G protein (Gx) mRNA |
| 69 | D76444 | Homo sapiens hkf-1 mRNA |
| 70 | D14041 | Homo sapiens mRNA for H-2K binding |
| | | factor-2 |
| 71 | X93209 | H.sapiens mRNA for NRD1 convertase |
| 72 | AF070644 | Homo sapiens clone 24742 mRNA |
| | | sequence |
| 73 | AB020713 | Homo sapiens mRNA for KIAA0906 |
| | | protein |
| 75 | D63476 | Human mRNA for KIAA0142 gene |
| 76 | N53547 | |
| 77 | L15388 | Human G protein-coupled receptor kinase |
| | | (GRK5) mRNA |
| 78 | X72308 | Homo sapiens mRNA for monocyte |
| | | chemotactic protein-3 (MCP-3) |
| 79 | X01703 | Human gene for alpha-tubulin (b alpha 1) |
| 80 | Y18004 | Homo sapiens mRNA for SCML2 protein |
| 81 | M27492 | Human interleukin 1 receptor mRNA |
| 82 | AL008729 | Human DNA sequence from PAC 257A7 |
| | | on chromosome 6p24 |
| 83 | D83776 | Human mRNA for KIAA0191 gene |
| 84 | AB011542 | Homo sapiens mRNA for MEGF9 |
| 85 | X68277 | H.sapiens CL 100 mRNA for protein |
| | | tyrosine phosphatase |
| 88 | Z99716 | Human DNA sequence from clone CTA- |
| | | 250D10 on chromosome 22 Contains the |
| | | genes for SREBF2 (sterol regulatory |
| | | element binding transcription factor 2), |
| | | NAGA (alpha-N-acetylgalactosaminidase), |
| | | a gene similar to neuronal-specific septin |
| | | 3, a pseudogene similar to ANT2 (adenine |
| | | nucleotide translocator 2), 2 mRNAs based |
| | | on ESTs, a genomic marker D22S1178, a |
| | | CA repeat polymorpism, ESTs and a CpG |
| | | island |
| 89 | X76648 | H.sapiens mRNA for glutaredoxin |
| 90 | W28281 | |
| 93 | U20938 | Human lymphocyte dihydropyrimidine |
| | | dehydrogenase mRNA |
| 94 | M63193 | Human platelet-derived endothelial cell |
| | | growth factor mRNA |
| 96 | X04412 | Human mRNA for plasma gelsolin |
| 97 | U77643 | Homo sapiens K12 protein precursor |
| | | mRNA |
| 99 | AF000424 | Homo sapiens LST1 mRNA, cLST1/C |
| | | splice variant |
| 100 | M29039 | Human transactivator (jun-B) gene |
| 102 | D38251 | Homo sapiens mRNA for RPB5 (XAP4) |
| 103 | X14787 | Human mRNA for thrombospondin |
| 106 | M16591 | Human hemopoietic cell protein-tyrosine |
| | | kinase (HCK) gene |
| 107 | AB015631 | Homo sapiens mRNA for type II |
| | | membrane protein |
| 108 | U51712 | |
| 109 | D86971 | Human mRNA for KIAA0217 gene |
| 110 | M55067 | Human 47-kD autosomal chronic |
| | | granulomatous disease protein mRNA |
| 111 | AB029036 | Homo sapiens mRNA for KIAA1113 |
| | | protein |
| 112 | AL050060 | Homo sapiens mRNA; cDNA |
| | | DKFZp566H073 (from clone |
| | | DKFZp566H073) |
| 113 | J04027 | Human plasma membrane Ca2⁺ pumping |
| | | ATPase mRNA |
| 114 | X57351 | Human 1-8D gene from interferon- |
| | | inducible gene family |
| 115 | M81750 | H.sapiens myeloid cell nuclear |
| | | differentiation antigen mRNA |
| 116 | U90546 | Human butyrophilin (BTF4) mRNA |
| 117 | AB018319 | Homo sapiens mRNA for KIAA0776 |
| | | protein |
| 118 | D82061 | Homo sapiens mRNA for a member of the |
| | | short-chain alcohol dehydrogenase family |
| 119 | AB014528 | Homo sapiens mRNA for KIAA0628 |
| | | protein |
| 120 | M25280 | Human lymph node homing receptor |
| | | mRNA |
| 121 | X76538 | H.sapiens Mpv17 mRNA |
| 123 | U13697 | Human interleukin 1-beta converting |
| | | enzyme isoform beta (IL1BCE) mRNA |
| 124 | X62055 | H.sapiens PTP1C mRNA for protein |
| | | tyrosine phosphatase 1C |
| 125 | AF051323 | Homo sapiens Src-associated adaptor |
| | | protein (SAPS) mRNA |
| 126 | M36820 | Human cytokine (GRO-beta) mRNA |
| 127 | AI201108 | |
| 128 | D14664 | Human mRNA for KIAA0022 gene |
| 131 | D14658 | Human mRNA for KIAA0102 gene |
| 132 | AA176780 | |
| 134 | U48213 | Human D-site binding protein gene |
| 135 | AF012086 | Homo sapiens Ran binding protein 2 |
| | | (RanBP2alpha) mRNA |
| 136 | U37518 | Human TNF-related apoptosis inducing |
| | | ligand TRAIL mRNA |
| 137 | AB020661 | Homo sapiens mRNA for KIAA0854 |
| | | protein |
| 138 | J05037 | Human serine dehydratase mRNA |
| 139 | AB011148 | Homo sapiens mRNA for KIAA0576 |
| | | protein |
| 140 | X16863 | Human Fc-gamma RIII-1 cDNA for Fc |
| | | gamma receptor III-1 (CD 16) |
| 141 | X06948 | Human mRNA for high affinity IgE |
| | | receptor alpha-subunit (FcERI) |
| 143 | X77956 | H.sapiens Id1 mRNA |
| 144 | AI760801 | |
| 145 | M34175 | Human beta adaptin mRNA |
| 146 | U68019 | Homo sapiens mad protein homolog |
| | | (hMAD-3) mRNA |
| 147 | AF006084 | Homo sapiens Arp2/3 protein complex |
| | | subunit p41-Arc (ARC41) mRNA |
| 149 | AL035079 | Human DNA sequence from clone 53C18 |
| | | on chromosome 11p12-13 |
| 150 | AF004563 | Homo sapiens hUNC18b alternatively |
| | | spliced mRNA |
| 151 | X52486 | Human mRNA for uracil-DNA |
| | | glycosylase |
| 152 | U15932 | Human dual-specificity protein |
| | | phosphatase mRNA |
| 153 | U20489 | Human glomerular epithelial protein 1 |
| | | (GLEPP1) mRNA |
| 154 | D89077 | Homo sapiens mRNA for Src-like adapter |
| | | protein |
| 155 | AJ224819 | Homo sapiens mRNA for candidate tumor |
| | | suppressor involved in B-CLL |
| 156 | X97324 | H.sapiens mRNA for adipophilin |
| 157 | AF002163 | Homo sapiens delta-adaptin mRNA |
| 158 | AL050025 | Homo sapiens mRNA; cDNA |
| | | DKFZp564D066 (from clone |
| | | DKFZp564D066) |
| 160 | M94345 | Homo sapiens macrophage capping protein |
| | | mRNA |
| 161 | AB016811 | Homo sapiens mRNA for ADP |
| | | ribosylation factor-like protein |
| 162 | S43855 | recoverin photoreceptor protein [human, |
| | | retina, mRNA] |
| 163 | M36821 | Human cytokine (GRO-gamma) mRNA |
| 164 | AF070606 | Homo sapiens clone 24411 mRNA |
| | | sequence |
| 165 | M62840 | Human acyloxyacyl hydrolase mRNA |
| 166 | AJ224442 | Homo sapiens mRNA for putative |
| | | methyltransferase |
| 167 | D13265 | Human mRNA for macrophage scavenger |
| | | receptor type II |
| 168 | U67322 | Human HBV associated factor (XAP4) |
| | | mRNA |
| 169 | U94333 | Human Clq/MBL/SPA receptor C1qR(p) |
| | | mRNA |
| 170 | L78440 | Homo sapiens STAT4 mRNA |
| 171 | AL049309 | Homo sapiens mRNA; cDNA |
| | | DKFZp564B176 (from clone |
| | | DKFZp564B176) |
| 172 | X55988 | Human EDN mRNA for eosinophil |
| | | derived neurotoxin |
| 173 | AL008637 | Human DNA sequence from clone CTA- |
| | | 833B7 on chromosome 22q12.3-13.2 |
| | | Contains the NCF4 gene for cytosolic |
| | | neutrophil factor 4 (40 kD), the 5′ part of |
| | | the CSF2RB gene for granulocyte- |
| | | macrophage low-affinity colony |
| | | stimulating factor 2 receptor beta, ESTs, |
| | | STSs and GSSs |
| 174 | M82809 | Human annexin IV (ANX4) mRNA |
| 176 | U72882 | Human interferon-induced leucine zipper |
| | | protein (IFP35) mRNA |
| 177 | AF054825 | Homo sapiens VAMP5 mRNA |
| 178 | AF014398 | Homo sapiens myo-inositol |
| | | monophosphatase 2 mRNA |
| 179 | U26174 | Human pre-granzyme 3 mRNA |
| 181 | U49392 | Human allograft inflammatory factor-1 |
| | | (AIF-1) mRNA |
| 182 | D83174 | Human mRNA for collagen binding |
| | | protein 2 |
| 183 | L33075 | Homo sapiens ras GTPase-activating-like |
| | | protein (IQGAP1) mRNA |
| 184 | AB020630 | Homo sapiens mRNA for KIAA0823 |
| | | protein |
| 185 | AI018098 | |
| 186 | Z22971 | H.sapiens mRNA for M130 antigen |
| | | extracellular variant |
| 187 | U51333 | Human hexokinase III (HK3) mRNA |
| 188 | K00650 | Human fos proto-oncogene (c-fos) |
| 189 | U49187 | Human placenta (Diff48) mRNA |
| 190 | L23134 | Homo sapiens metase (MET-1) mRNA |
| 191 | X61118 | Human TTG-2 mRNA for a cysteine rich |
| | | protein with LIM motif |
| 192 | AB023211 | Homo sapiens mRNA for KIAA0994 |
| | | protein |
| 193 | AL031282 | Human DNA sequence from clone 283E3 |
| | | on chromosome 1p36.21-36.33. Contains |
| | | the alternatively spliced gene for Matrix |
| | | Metalloproteinase in the Female |
| | | Reproductive tract MIFR1, -2, |
| | | MMP21/22A, -B and -C, a novel gene, the |
| | | alternatively spliced CDC2L2 gene for |
| | | Cell Division Cycle 2-Like 2 (PITSLRE, |
| | | p58/GTA, Galactosyltransferase |
| | | Associated Protein Kinase) beta 1, beta 2- |
| | | 1, beta 2-2 and alpha 2-4, a 40S Ribosomal |
| | | Protein S7 pseudogene, part of the |
| | | KIAA0447 gene, a novel alternatively |
| | | spliced gene similar to many |
| | | (archae)bacterial, worm and yeast |
| | | hypothetical genes, and the GNB1 gene for |
| | | Guanine Nucleotide Binding Protein (G |
| | | protein), Beta polypeptide 1 (Transducin |
| | | Beta chain 1). Contains putative CpG |
| | | islands, ESTs, STSs and GSSs |
| 194 | X15376 | Human mRNA for GABA-A receptor, |
| | | gamma 2 subunit |
| 195 | AB007864 | Homo sapiens KIAA0404 mRNA |
| 196 | U12471 | Human thrombospondin-1 gene |
| 197 | S71043 | Ig alpha 2 mmunoglobulin A heavy chain |
| | | allotype 2 {constant region, germ line} |
| | | [human, peripheral blood neutrophils, |
| | | Genomic] |
| 198 | M14758 | Homo sapiens P-glycoprotein (PGY1) |
| | | mRNA |
| 199 | X84373 | H.sapiens mRNA for nuclear factor |
| | | RIP140 |
| 200 | M34423 | Human beta-galactosidase (GLB1) mRNA |
| 201 | AF087036 | Homo sapiens musculin mRNA |
| 202 | U95006 | Human D9 splice variant A mRNA |
| 203 | AA631972 | |
| 204 | Z24724 | H.sapiens polyA site DNA |
| 205 | AF055008 | Homo sapiens clone 24720 epithelin 1 and |
| | | 2 mRNA |
| 206 | D30783 | Homo sapiens mRNA for epiregulin |
| 208 | AI142565 | |
| 210 | M10098 | Human 18S rRNA gene |
| 211 | U82303 | Homo sapiens unknown protein mRNA |
| 212 | L25081 | Homo sapiens GTPase (rhoC) mRNA |
| 214 | M13755 | Human interferon-induced 17-kDa/15-kDa |
| | | protein mRNA |
| 216 | M13142 | Human factor XI (blood coagulation |
| | | factor) mRNA |
| 217 | AI687419 | |
| 219 | AI961743 | |
| 220 | X93086 | H.sapiens mRNA for biliverdin IX alpha |
| | | reductase |
| 221 | J04765 | Human osteopontin mRNA |
| 222 | L37792 | Homo sapiens syntaxin 1A mRNA |
| 223 | X91817 | H.sapiens mRNA for transketolase-like |
| | | protein |
| 224 | S81914 | IEX-1 = radiation-inducible immediate- |
| | | early gene [human, placenta, mRNA] |
| 225 | X02419 | H.sapiens uPA gene |
| 226 | X01630 | Human mRNA for argininosuccinate |
| | | synthetase |
| 227 | AJ001481 | Homo sapiens mRNA for DUX1 protein |
| 228 | U79725 | Human A33 antigen precursor mRNA |
| 229 | M14083 | Human beta-migrating plasminogen |
| | | activator inhibitor I mRNA |
| 230 | U03688 | Human dioxin-inducible cytochrome P450 |
| | | (CYP1B1) mRNA |
| 231 | U67963 | Human lysophospholipase homolog (HU |
| | | K5) mRNA |
| 232 | AJ223183 | Homo sapiens mRNA for DORA protein |
| 233 | AB018549 | Homo sapiens MD-2 mRNA |
| 234 | U07563 | Human ABL gene, exon 1b and intron 1b, |
| | | and putative M8604 Met protein (M8604 |
| | | Met) gene |
| 235 | AI961220 | |
| 236 | S82470 | BB1 malignant cell expression-enhanced |
| | | gene/tumor progression-enhanced gene |
| | | [human, UM-UC-9 bladder carcinoma cell |
| | | line, mRNA] |
| 237 | Y14737 | Homo sapiens mRNA for immunoglobulin |
| | | lambda heavy chain |
| 240 | Y09392 | H.sapiens mRNA for WSL-LR, WSL-S1 |
| | | and WSL-S2 proteins |
| 241 | AL031685 | Human DNA sequence from clone RP5- |
| | | 963K23 on chromosome 20q13.1 1-13.2 |
| | | Contains a KRT18 (Keratin type I, |
| | | Cytoskeletal 18 (Cytokeratin 18, |
| | | CK18,CYK18)) pseudogene, a gene for a |
| | | novel protein, the SPATA2 gene for |
| | | spermatogenesis associated protein 2 |
| | | (KIAA0757) and the 3′ end of the gene for |
| | | KIAA0939 (novel Sodium/hydrogen |
| | | exchanger family member). Contains |
| | | ESTs, STSs, GSSs and four putative CpG |
| | | islands |
| 242 | AL049963 | Homo sapiens mRNA; cDNA |
| | | DKFZp564A132 (from clone |
| | | DKFZp564A132) |
| 243 | AL022162 | Homo sapiens DNA sequence from PAC |
| | | 454M7 on chromosome Xq25-26.3. |
| | | Contains the OCRL1 gene for Lowe |
| | | Oculocerebrorenal Syndrome protein |
| | | OCRL-1. Contains ESTs, STSs and GSSs |
| 244 | AF022375 | Homo sapiens vascular endothelial growth |
| | | factor mRNA |
| 245 | U29943 | Human ELAV-like neuronal protein-2 |
| | | Hel-N2 mRNA |
| 246 | M64929 | Human protein phosphatase 2A alpha |
| | | subunit mRNA |
| 247 | AF001862 | Homo sapiens FYN binding protein |
| | | mRNA |
| 248 | X04371 | Human 1.6 Kb mRNA for 2-5A synthetase |
| | | induced by interferon |
| 249 | X04011 | Human mRNA of X-CGD gene involved |
| | | in chronic granulomatous disease located |
| | | on chromosome X |
| 250 | AB016789 | Homo sapiens mRNA for |
| | | Glutamine:fructose-6-phosphate |
| | | amidotransferase |
| 251 | AF002697 | Homo sapiens E1B 19K/Bc1-2-binding |
| | | protein Nip3 mRNA, nuclear gene |
| | | encoding mitochondrial protein |
| 252 | D21878 | Human mRNA for BST-1 |
| 253 | U50535 | Human BRCA2 region, mRNA sequence |
| | | CG006 |
| 254 | U20350 | Human C protein-coupled receptor V28 |
| | | mRNA |
| 255 | AF045800 | Homo sapiens gremlin mRNA |
| 256 | D89974 | Homo sapiens mRNA for |
| | | glycosylphosphatidyl inositol-anchored |
| | | protein GPI-80 |
| 257 | D31764 | Human mRNA for KIAA00064 gene |
| 258 | X54489 | Human gene for melanoma growth |
| | | stimulatory activity (MGSA) |
| 259 | AB014555 | Homo sapiens mRNA for KIAA0655 |
| | | protein |
| 260 | M33647 | Human retinoblastoma associated (RB1) |
| | mRNA | |
| 261 | D38552 | Human mRNA for KIAA0073 gene |
| 262 | X04106 | Human mRNA for calcium dependent |
| | | protease (small subunit) |
| 263 | U92818 | Homo sapiens c33.28 unnamed HERV-H |
| | | protein mRNA |
| 264 | AF010316 | Homo sapiens Pig12 (PIG12) mRNA |
| 265 | X54486 | Human gene for C1-inhibitor |
| 266 | D64142 | Human mRNA for histone H1x |
| 267 | M15330 | Human interleukin 1-beta (IL1B) mRNA |
| 268 | J02931 | Human placental tissue factor (two forms) |
| | | mRNA |
| 269 | U48807 | Human MAP kinase phosphatase (MKP-2) |
| | | mRNA |
| 270 | M27826 | Homo sapiens human endogeneous |
| | | retrovirus RTVL-H neutral protease large |
| | | subunit mRNA |
| 271 | AL049250 | Homo sapiens mRNA; cDNA |
| | | DKFZp564D113 (from clone |
| | | DKFZp564D113) |
| 272 | AB002308 | Homo sapiens mRNA for KIAA0310 |
| | | protein |
| 275 | AI817548 | |
| 276 | M28130 | Human interleukin 8 (IL8) gene |
| 278 | Z38026 | H.sapiens mRNA for FALL-39 peptide |
| | | antibiotic |
| 280 | D49357 | Human mRNA for S-adenosylmethionine |
| | | synthetase |
| 281 | J02871 | |
| | | Human lung cytochrome P450 (IV |
| | | subfamily) BI protein |
| 282 | M55405 | Homo sapiens mucin (MUC-3) mRNA |
| 283 | W27095 | |
| 284 | U64197 | Homo sapiens chemokine exodus-1 |
| | | mRNA |
| 285 | X52015 | H.sapiens mRNA for interleukin-1 |
| | | receptor antagonist |
| 286 | X95808 | H.sapiens mRNA for protein encoded by a |
| | | candidate gene, DXS6673E, for mental |
| | | retardation |
| 288 | AF027516 | Homo sapiens trans-golgi network |
| | | glycoprotein 51 (TGN) mRNA |

CI-779 activity genes represented by each qualifier in Table 2 can be identified based on the HG-U95Av2 gene chip annotation provided by Affymetrix. Genes thus identified are illustrated in Table 5. CCI-779 activity genes can also be determined based on the corresponding Entrez accession numbers. In addition, CCI-779 activity genes can be determined by BLAST searching the corresponding CPSs, or the unambiguous segments of the corresponding SEQ ID NOs, against a human genome sequence database. Suitable human genome sequence databases for this purpose include, but are not limited to, the NCBI human genome database. The NCBI also provides BLAST programs, such as “blastn,” for searching its sequence databases.

TABLE 5
CCI-779 Activity Genes

| | | |
|---|---|---|
| | | Sequences Useful For Making Probes/Primers |
| CPS | CCI-779 Activity Gene | for CCI-779 Activity Genes |

| | | |
|---|---|---|
| 1 | DKFZP564E1962 | SEQ ID NO: 1 |
| 2 | ETR101 | SEQ ID NO: 2 |
| 3 | PFN1 | SEQ ID NO: 3 |
| 4 | APOBECIL | SEQ ID NO: 4 |
| 5 | CKAP1 | SEQ ID NO: 5 |
| 6 | KIAA0220 | SEQ ID NO: 6 |
| 7 | CORO1A | SEQ ID NO: 7 |
| 8 | GNL1 | SEQ ID NO: 8 |
| 9 | AKR1A1 | SEQ ID NO: 9 |
| 10 | ID2 | SEQ ID NOS: 10, 289, and 353 |
| 11 | PSG7 | SEQ ID NO: 11 |
| 13 | IDH2 | SEQ ID NO: 12 |
| 14 | RER1 | SEQ ID NO: 13 |
| 15 | TUBB | SEQ ID NOS: 14 and 290 |
| 16 | HADH2 | SEQ ID NO: 15 |
| 17 | NDUFB5 | SEQ ID NO: 16 |
| 18 | APRT | SEQ ID NOS: 17 and 310 (Y00486) |
| 19 | PSME2 | SEQ ID NOS: 18 and 291 |
| 20 | MTCP1 | SEQ ID NOS: 19 and 311 (Z24459) |
| 21 | NDUFB3 | SEQ ID NOS: 20 and 312 (A203354) |
| 23 | CTSL | SEQ ID NO: 21 |
| 24 | FGL2 | SEQ ID NOS: 22 and 292 |
| 25 | DKFZP564C1940 | SEQ ID NO: 23 |
| 26 | 28SRNA5_Hs_AFFX | SEQ ID NO: 24 |
| 27 | SEMA3F | SEQ ID NO: 25 |
| 28 | BST2 | SEQ ID NO: 26 |
| 29 | UNK_AL050021 | SEQ ID NO: 27 |
| 30 | UBE2L6 | SEQ ID NO: 28 |
| 31 | SLC11A1 | SEQ ID NOS: 29 and 354 |
| 32 | CDKN1C | SEQ ID NOS: 30 and 313 (U22398) |
| 33 | GAIP | SEQ ID NO: 31 |
| 34 | TACC2 | SEQ ID NOS: 32 and 355 |
| 35 | CLNS1A | SEQ ID NO: 33 |
| 36 | DPYSL2 | SEQ ID NO: 34 |
| 37 | FCGR3A | SEQ ID NO: 35 |
| 38 | UQCRC1 | SEQ ID NO: 36 |
| 39 | GZMA | |
| 41 | PSMB10 | SEQ ID NOS: 38 and 314 (X71874) |
| 42 | IDS | SEQ ID NO: 39 |
| 43 | AHNAK | SEQ ID NOS: 40, 315 (M80899), and 356 |
| 44 | PTPN7 | SEQ ID NOS: 41 and 293 |
| 45 | DUSP2 | SEQ ID NO: 42 |
| 46 | PSMB9 | SEQ ID NO: 43 |
| 47 | UNK_AI652978 | SEQ ID NO:44 |
| 48 | UNK_AF025529 | SEQ ID NO: 45 |
| 49 | SDR1 | SEQ ID NO: 46 |
| 50 | POLR2L | SEQ ID NO: 47 |
| 51 | SCYA2 | SEQ ID NOS: 48 and 294 |
| 52 | PAI2 | SEQ ID NO: 49 |
| 53 | UNK_U66047 | SEQ ID NO: 50 |
| 54 | UNK_U00930 | SEQ ID NO: 51 |
| 55 | JMJ | SEQ ID NO: 52 |
| 56 | UGP2 | SEQ ID NOS: 53 and 357 |
| 57 | KIAA0410 | SEQ ID NO: 54 |
| 58 | NDUFS7 | SEQ ID NOS: 55 and 316 (AC005329) |
| 59 | KIAA0645 | SEQ ID NO: 56 |
| 60 | GSTP1 | SEQ ID NOS: 57, 295, and 317 (U12472) |
| 61 | DECR1 | SEQ ID NOS: 58 and 318 (U78302) |
| 62 | PLXNC1 | SEQ ID NOS: 59 and 319 (AF030339) |
| 63 | TUBA2 | SEQ ID NOS: 60 and 320 (AF005392) |
| 64 | NME1 | SEQ ID NO: 61 |
| 65 | UNK_AI679353 | SEQ ID NO: 62 |
| 66 | RNAHP | SEQ ID NOS: 63 and 321 (H68340) |
| 67 | ZNF198 | SEQ ID NO: 64 |
| 68 | PRCP | SEQ ID NO: 65 |
| 70 | DKFZP586A0522 | SEQ ID NO: 66 |
| 71 | SFRS11 | SEQ ID NO: 67 |
| 72 | RAC2 | SEQ ID NO: 68 |
| 73 | ZFP103 | SEQ ID NO: 69 |
| 74 | LOC51580 | SEQ ID NOS: 70 and 358 |
| 75 | NRD1 | SEQ ID NOS: 71 and 359 |
| 76 | UNK_AF070644 | SEQ ID NO: 72 |
| 77 | KIAA0906 | SEQ ID NO: 73 |
| 78 | MT1F | SEQ ID NOS: 74 and 322 (M10943) |
| 79 | P85SPR | SEQ ID NO: 75 |
| 80 | UNK_N53547 | SEQ ID NO: 76 |
| 81 | GPRK5 | SEQ ID NOS: 77 and 296 |
| 83 | SCYA7 | SEQ ID NO: 78 |
| 84 | TUBA3 | SEQ ID NO: 79 |
| 85 | SCML2 | SEQ ID NO: 80 |
| 87 | IL1R1 | SEQ ID NO: 81 |
| 88 | UNK_AL008729 | SEQ ID NO: 82 |
| 89 | KIAA0191 | SE ID NO: 83 |
| 90 | EGFL5 | SEQ ID NO: 84 |
| 91 | DUSP1 | SEQ ID NO: 85 |
| 92 | FBP1 | SEQ ID NOS: 86 and 323 (U21931) |
| 93 | HRB | SEQ ID NOS: 87 and 324 (L42025) |
| 94 | NAGA | SEQ ID NO: 88 |
| 95 | GLRX | SE ID NO: 89 |
| 96 | UNK_W28281 | SEQ ID NOS: 90 and 360 |
| 97 | UNK_AL096740 | SEQ ID NOS: 91, 325 (AL096740), and 361 |
| 98 | TBXAS1 | SEQ ID NOS: 92, 326 (D34625), 362, and 363 |
| 99 | DPYD | |
| 100 | ECGF1 | SEQ ID NOS: 94 and 297 |
| 101 | TIMP1 | SEQ ID NOS: 95 and 327 (D11139) |
| 102 | GSN | SEQ ID NO: 96 |
| 103 | SECTM1 | SEQ ID NO: 97 |
| 104 | OLR1 | SEQ ID NOS: 98 and 328 (AF079167) |
| 105 | D6S49E | SEQ ID NOS: 99 and 364 |
| 106 | JUNB | SEQ ID NO: 100 |
| 107 | PFC | SEQ ID NOS: 101 and 329 (AF005664) |
| 108 | POLR2E | SEQ ID NO: 102 |
| 109 | THBS1 | SEQ ID NO: 103 |
| 110 | HK2 | SEQ ID NOS: 104 and 330 (Z46376) |
| 111 | INSIG1 | SEQ ID NOS: 105, 331 (U96876), 365, and 366 |
| 112 | HCK | SEQ ID NOS: 106 and 298 |
| 113 | HP10390 | SEQ ID NO: 107 |
| 114 | UNK_U51712 | SEQ ID NO: 108 |
| 115 | KIAA0217 | SEQ ID NO: 109 |
| 116 | NCF1 | SEQ ID NO: 110 |
| 117 | KIAA1113 | SEQ ID NOS: 111 and 367 |
| 118 | DKFZP566H073 | SEQ ID NO: 112 |
| 119 | ATP2B1 | SEQ ID NOS: 113 and 368 |
| 120 | IFITM2 | SEQ ID NO: 114 |
| 121 | MNDA | SEQ ID NO: 115 |
| 122 | BTN3A2 | SEQ ID NO: 116 |
| 123 | KIAA0776 | SEQ ID NO: 117 |
| 124 | D6S2245E | SEQ ID NO: 118 |
| 125 | KIAA0628 | SEQ ID NO: 119 |
| 126 | SELL | SEQ ID NO: 120 |
| 127 | MPV17 | SEQ ID NO: 121 |
| 128 | FABP5 | SEQ ID NOS: 122 and 332 (M94856) |
| 129 | CASP1 | SEQ ID NOS: 123 and 299 |
| 130 | PTPN6 | SEQ ID NO: 124 |
| 131 | SKAP-HOM | SEQ ID NO: 125 |
| 133 | GRO2 | SEQ ID NO: 126 |
| 134 | RRAS | SEQ ID NO: 127 |
| 135 | KIAA0022 | SEQ ID NOS: 128 and 369 |
| 136 | FLII | SEQ ID NOS: 129 and 333 (U80184) |
| 137 | PRF1 | SEQ ID NOS: 130 and 334 (M28393) |
| 138 | KIAA0102 | SEQ ID NO: 131 |
| 139 | UNK_AA176780 | SEQ ID NO: 132 |
| 140 | PYGL | SEQ ID NOS: 133 and 335 (AF046798) |
| 141 | DBP | SEQ ID NO: 134 |
| 143 | RANBP2L1 | SEQ ID NO: 135 |
| 144 | TNFSF10 | SEQ ID NO: 136 |
| 145 | KIAA0854 | SEQ ID NO: 137 |
| 146 | SDS | SEQ ID NOS: 138 and 370 |
| 147 | KIAA0576 | SEQ ID NO: 139 |
| 148 | FCGR3B | SEQ ID NO: 140 |
| 149 | FCER1A | SEQ ID NO: 141 |
| 150 | CD44 | SEQ ID NOS: 142 and 336 (L05424) |
| 151 | ID1 | SEQ ID NOS: 143 and 300 |
| 152 | UNK_AI760801 | SEQ ID NO: 144 |
| 153 | ADTB2 | SEQ ID NO: 145 |
| 154 | MADH3 | SEQ ID NOS: 146 and 301 |
| 155 | ARPC1B | SEQ ID NO: 147 |
| 156 | TALDO1 | SEQ ID NOS: 148, 337 (AF010400), and 371 |
| 157 | UNK_AL035079 | SEQ ID NO: 149 |
| 158 | STXBP1 | SEQ ID NO: 150 |
| 159 | UNG2 | SEQ ID NO: 151 |
| 160 | DUSP5 | SEQ ID NO: 152 |
| 161 | PTPRO | SEQ ID NO: 153 |
| 162 | SLA | SEQ ID NOS: 154 and 302 |
| 163 | RFP2 | SEQ ID NO: 155 |
| 164 | ADFP | SEQ ID NOS: 156 and 372 |
| 165 | ADTD | SEQ ID NOS: 157 and 373 |
| 166 | ADTG | SEQ ID NO: 158 |
| 168 | UNK_X87344 | SEQ ID NOS: 159 and 338 (X87344) |
| 170 | CAPG | SEQ ID NO: 160 |
| 171 | ARL7 | SEQ ID NO: 161 |
| 172 | RCV1 | SEQ ID NO: 162 |
| 173 | GRO3 | SEQ ID NO: 163 |
| 175 | UNK_AF070606 | SEQ ID NO: 164 |
| 176 | AOAH | SEQ ID NO: 165 |
| 177 | UNK_AJ224442 | SEQ ID NO: 166 |
| 178 | MSR1 | SEQ ID NO: 167 |
| 179 | XAP4 | SEQ ID NO: 168 |
| 180 | C1QR | SEQ ID NO: 169 |
| 181 | STAT4 | SEQ ID NO: 170 |
| 182 | UNK_AL049309 | SEQ ID NO: 171 |
| 183 | RNASE2 | SEQ ID NO: 172 |
| 184 | NCF4 | SEQ ID NOS: 173 and 303 |
| 185 | ANXA4 | SE ID NO: 174 |
| 186 | ME2 | SEQ ID NOS: 175 and 339 (M55905) |
| 187 | IFI35 | SEQ ID NO: 176 |
| 188 | VAMP5 | SEQ ID NO: 177 |
| 189 | IMPA2 | SEQ ID NO: 178 |
| 190 | GZMK | SEQ ID NO: 179 |
| 191 | BGN | SEQ ID NOS: 180 and 340 (J04599) |
| 192 | AIF1 | SEQ ID NO: 181 |
| 193 | CBP2 | SEQ ID NO: 182 |
| 195 | IQGAP1 | SEQ ID NO: 183 |
| 196 | KIAA0823 | SEQ ID NO: 184 |
| 197 | UNK_AI018098 | SEQ ID NOS: 185 and 374 |
| 198 | CD163 | SEQ ID NO: 186 |
| 199 | HK3 | SEQ ID NO: 187 |
| 200 | FOS | SEQ ID NO: 188 |
| 201 | DIFF48 | SEQ ID NO: 189 |
| 202 | UNK_L23134 | SEQ ID NO: 190 |
| 203 | LMO2 | SEQ ID NO: 191 |
| 204 | PDI2 | SEQ ID NO: 192 |
| 205 | UNK_AL031282 | SEQ ID NO: 193 |
| 206 | GABRG2 | SEQ ID NO: 194 |
| 207 | KIAA0404 | SEQ ID NO: 195 |
| 208 | UNK_U12471 | SEQ ID NO: 196 |
| 209 | IGHA1 | SEQ ID NOS: 197, 304, and 305 |
| 210 | ABCB1 | SEQ ID NO: 198 |
| 211 | NRIP1 | SEQ ID NO: 199 |
| 213 | GLB1 | SEQ ID NO: 200 |
| 214 | MSC | SEQ ID NO: 201 |
| 216 | UNK_U95006 | SEQ ID NO: 202 |
| 217 | NK4 | SEQ ID NO: 203 |
| 218 | UNK_Z24724 | SEQ ID NO: 204 |
| 219 | GRN | SEQ ID NO: 205 |
| 220 | EREG | SEQ ID NO: 206 |
| 221 | KIAA0382 | SEQ ID NOS: 207 and 341 (AB002380) |
| 222 | RNASE6 | SEQ ID NO: 208 |
| 223 | KIAA0442 | SEQ ID NOS: 209, 342 (AB007902), and 375 |
| 225 | 18SRNA3_Hs_AFFX | SEQ ID NO: 210 |
| 226 | UNK_U82303 | SEQ ID NO: 211 |
| 227 | ARHC | SEQ ID NO: 212 |
| 228 | CREM | SEQ ID NOS: 213, 306, and 343 (S68134) |
| 229 | ISG15 | SEQ ID NO: 214 |
| 230 | CCR7 | SEQ ID NOS: 215 and 344 (L31584) |
| 231 | F11 | SEQ ID NOS: 216 and 376 |
| 232 | UNK_AI687419 | SEQ ID NO: 217 |
| 233 | UNK_AC002073 | SEQ ID NOS: 218, 345 (AC002073), and 377 |
| 234 | STK17A | SEQ ID NO: 219 |
| 235 | BLVRA | SEQ ID NO: 220 |
| 236 | SPP1 | SEQ ID NOS: 221 and 307 |
| 237 | STX1A | SEQ ID NO: 222 |
| 238 | TKTL1 | SEQ ID NO: 223 |
| 239 | IER3 | SEQ ID NO: 224 |
| 241 | PLAU | SEQ ID NO: 225 |
| 242 | ASS | SEQ ID NO: 226 |
| 244 | DUX1 | SEQ ID NO: 227 |
| 245 | GPA33 | SEQ ID NO: 228 |
| 246 | PAI1 | SEQ ID NO: 229 |
| 247 | CYP1B1 | SEQ ID NOS: 230 and 308 |
| 249 | HU-K5 | SEQ ID NOS: 231 and 378 |
| 250 | DORA | SEQ ID NO: 232 |
| 253 | MD-2 | SEQ ID NO: 233 |
| 254 | UNK_U07563 | SEQ ID NO: 234 |
| 255 | SPINK1 | SEQ ID NO: 235 |
| 256 | UNK_S82470 | SEQ ID NO: 236 |
| 257 | IGHG3 | SEQ ID NO: 237 |
| 258 | UNK_AA151971 | SEQ ID NOS: 238, 309, 346 (AA151971), and 379 |
| 259 | UNK_AA216639 | SEQ ID NOS: 239 and 347 (AA216639) |
| 260 | TNFRSF12 | SEQ ID NO: 240 |
| 261 | UNK_AL031685 | SEQ ID NO: 241 |
| 262 | UNK_AL049963 | SEQ ID NO: 242 |
| 263 | APELIN | SEQ ID NO: 243 |
| 264 | VEGF | SEQ ID NO: 244 |
| 265 | ELAVL2 | SEQ ID NO: 245 |
| 266 | PPP2R2A | SEQ ID NO: 246 |
| 267 | UNK_AF001862 | SEQ ID NOS: 247 and 380 |
| 268 | OAS1 | SEQ ID NO: 248 |
| 269 | CYBB | SEQ ID NO: 249 |
| 270 | GFPT2 | SEQ ID NO: 250 |
| 271 | BNIP3 | SEQ ID NO: 251 |
| 272 | BST1 | SEQ ID NO: 252 |
| 273 | UNK_U50535 | SEQ ID NO: 253 |
| 274 | CX3CR1 | SEQ ID NO: 254 |
| 275 | CKTSF1B1 | SEQ ID NO: 255 |
| 276 | VNN2 | SEQ ID NO: 256 |
| 277 | KIAA0064 | SEQ ID NOS: 257 and 381 |
| 278 | GRO1 | SEQ ID NO: 258 |
| 279 | KIAA0655 | SEQ ID NO: 259 |
| 280 | RB1 | SEQ ID NO: 260 |
| 281 | KIAA0073 | SEQ ID NO: 261 |
| 282 | CAPN4 | SEQ ID NO: 262 |
| 283 | UNK_U92818 | SEQ ID NO: 263 |
| 284 | MGST1L1 | SEQ ID NO:264 |
| 285 | C1NH | SEQ ID NO: 265 |
| 286 | H1FX | SEQ ID NO: 266 |
| 287 | IL1B | SEQ ID NO 267 |
| 288 | F3 | SEQ ID NO: 268 |
| 289 | DUSP4 | SEQ ID NO: 269 |
| 290 | HUMRTVLH3 | SEQ ID NO: 270 |
| 291 | UNK_AL049250 | SEQ ID NO: 271 |
| 292 | UNK_AB002308 | SEQ ID NO: 272 |
| 293 | EDN1 | SEQ ID NOS: 273 and 348 (J05008) |
| 295 | FRAT2 | SEQ ID NOS: 274 and 349 (AF062739) |
| 296 | UNK_AI817548 | SEQ ID NOS: 275 and 382 |
| 297 | IL8 | SEQ ID NO: 276 |
| 298 | FSCN2 | SEQ ID NOS: 277, 350 (AI189621), and 383 |
| 300 | CAMP | SEQ ID NO: 278 |
| 301 | FCGR1A | SEQ ID NOS: 279 and 351 (M63835) |
| 302 | MAT1A | SEQ ID NOS: 280 and 384 |
| 303 | CYP4B1 | SEQ ID NO: 281 |
| 304 | MUC3 | SEQ ID NO: 282 |
| 305 | B7 | SEQ ID NO: 283 |
| 306 | SCYA20 | SEQ ID NO: 284 |
| 307 | IL1RN | SEQ ID NO: 285 |
| 308 | ZNF261 | SEQ ID NO: 286 |
| 309 | LGALS2 | SEQ ID NOS: 287, 352 (AL022315), and 385 |
| 310 | TGN51 | SEQ ID NOS: 288 and 386 |

In one embodiment, the BLAST search of the NCBI human genome database is conducted by using CPSs. Gene(s) that aligns to a given CPS with at least 95% sequence identity can be identified. In many cases, the identified gene(s) has at least 96%, 97%, 98%, 99%, or more sequence identity with the CPS. The results of the BLAST search are detailed below.

CPS 1 corresponds to DKFZP564E1962 (TXNDC) which encodes thioredoxin domain-containing. This gene has LocusID: 81542, and is located on chromosome 14 with reported cytogenetic location 14q21.3. The gene resides in genomic locus NT_{—}025892 (NCBI Genome Annotation). The gene product is a member of the thioredoxin family.

CPS 1 also has 86-90% sequence identity with an intron sequence of GK003 which encodes GK003 protein. GK003 has LocusID: 57002, and is located on chromosome 7 with reported cytogenetic location 7p15.2. In addition, fragments of CPS 1 align with a chromosomal region on chromosome 13 and an intron sequence of OATPRP4 with 87-95% sequence identity. The chromosomal region on chromosome 13 is located near ING1 which encodes inhibitor of growth family, member 1, and has LocusID: 3621. OATPRP4 encodes organic anion transporter polypeptide-related protein 4, and has LocusID: 81796 with reported cytogenetic location 8q13.1.

CPS 2 corresponds to ETR101 which encodes immediate early protein. This gene has LocusID: 9592, and is located on chromosome 19 with reported cytogenetic location 19 p13.13. The gene resides in genomic locus NT_{—}031915 (NCBI Genome Annotation). Expression of immediate early protein can be induced by TPA stimulation in promyelocytic leukemia cell line HL-60 and in other leukemia cell lines.

A fragment of CPS 2 (nucleotides 309 to 492 of CPS 2) shows 98% sequence identity with an intron sequence of LOC169782. LOC169782 encodes a protein similar to transcription factor IIIA (Factor A) (TFIIIA), and has reported cytogenetic location 9p24.1.

CPS 3 corresponds to PFN1 which encodes profilin 1. This gene has LocusID: 5216, and is located on chromosome 17 with reported cytogenetic location 17 p13.3. The gene resides in genomic locus NT_{—}033299 (NCBI Genome Annotation). Profilin 1 is a ubiquitous actin monomer-binding protein belonging to the profilin family. It is thought to regulate actin polymerization in response to extracellular signals. Deletion of PFN 1 gene is associated with Miller-Dieker syndrome.

Nucleotides 7 to 406 of CPS 3 align with various regions in the human genome with 86-93% sequence identity. These regions include LOC163511, COAS3, a region near LOC149010, a region near LOC200030, an intron sequence of DKFZp434D177, FLJ20719, LOC199970, and LOC206456. LOC163511 encodes a protein similar to profilin I, and has reported cytogenetic location 1q23.2. COAS3 encodes chromosome 1 amplified sequence 3, and has LocusID: 200025 with reported cytogenetic location 1q12. LOC149010 encodes a protein similar to hypothetical protein DKFZp434D177, and has reported cytogenetic location 1q12. LOC200030 encodes a protein similar to hypothetical protein DJ328E19.C1.1, and has reported cytogenetic location 1q12. DKFZp434D177 encodes hypothetical protein DKFZp434D177, and has LocusID: 84224 with reported cytogenetic location 1p36.12. FLJ20719 encodes hypothetical protein FLJ20719, and has LocusID: 55672 with reported cytogenetic location 1p31. LOC199970 has reported cytogenetic location 1p11.1. LOC206456 encodes a protein similar to chain P, structure of bovine beta-actin-profilin complex with actin bound Atp phosphates solvent accessible. LOC206456 is located on chromosome 6.

CPS 4 corresponds to APOBEC 1L (APOBEC3C) which encodes a protein similar to APOBECI protein. APOBECIL gene has LocusID: 27350, and is located on chromosome 22 with reported cytogenetic location 22q13.1-q13.2. APOBECIL gene product is similar to phorbolin (DJ742C19.2), and may catalyze hydrolytic deamination of cytidine nucleotides. The gene product contains a cytidine deaminase zinc-binding domain.

CPS 4 also has 91% sequence identity with LOC200316 which encodes a protein similar to phorbolin 3 (APOBECI-like). LOC200316 has LocusID: 200316 with reported cytogenetic location 22q13.1. In addition, CPS 4 aligns with two other regions on chromosome 22 with 89-92% sequence identity.

CPS 5 corresponds to CKAP1 which encodes cytoskeleton-associated protein 1. This gene has LocusID: 1155, and is located on chromosome 19 with reported cytogenetic location 19q13.11-q13.12. The gene resides in genomic locus NT_{—}011296 (NCBI Genome Annotation). Cytoskeleton-associated protein 1 associates with microtubules. It contains a glycine domain which plays a role in association with microtubules.

Affymetrix annotation suggests that CPS 6 corresponds to KIAA0220 which has LocusID: 23117 and is located at chromosome 16 p12.1. Blast search of the Entrez human genome sequence database shows that CPS 6 has 99% sequence identity to a region on chromosome 16. This region is near LOC255565, and resides in genomic locus NT_{—}035368 (NCBI Genome Annotation).

In addition, fragments of CPS 6 align with various chromosomal regions with at least 90% sequence identity. These regions include LOC220555, LOC124302, a chromosomal region near LOC220567, a chromosomal region near LOC254081, and a chromosomal region near LOC197363. LOC220555 encodes a protein similar to nuclear pore complex interacting protein, and has reported cytogenetic location 16p 11.2. LOC124302 encodes a protein similar to nuclear pore complex interacting protein, and has reported cytogenetic location 18 p11.1. LOC220567 encodes a protein similar to apolipoprotein B48 receptor, and is located at chromosome 16q13. LOC254081 encodes a protein similar to group X secretory phospholipase A2 precursor (phosphatidylcholine 2-acylhydrolase GX) (GX sPLA2) (sPLA2-X), and is located on chromosome 16. LOC197363 encodes a protein similar to ataxin 2 related protein (isoform 1), and is located on chromosome 16.

CPS 7 corresponds to CORO1A which encodes coronin, actin binding protein, 1A. This gene has LocusID: 11151, and is located on chromosome 16 with reported cytogenetic location 16q13. The gene resides in genomic locus NT_{—}033291 (NCBI Genome Annotation). The gene product (coronin 1A) binds to actin, and may be involved in mitosis, cell motility, formation of phagocytic vacuoles and phagocytosis. Coronin 1A has at least five WD domains.

CPS 8 corresponds to GNL1 which encodes guanine nucleotide binding protein-like 1. This gene has LocusID: 2794, and is located on chromosome 6 with reported cytogenetic location 6p21.3. The gene resides in genomic locus NT_{—}007592 (NCBI Genome Annotation). The GNL1 gene, identified in the human major histocompatibility complex class I region, shows a high degree of similarity with its mouse counterpart. The GNL1 gene is located less than 2 kb centromeric to HLA-E, in the same transcriptional orientation. GNL1 is telomeric to HLA-B and HLA-C.

CPS 9 corresponds to AKR1A1 which encodes aldo-keto reductase family 1, member Al (aldehyde reductase). This gene has LocusID: 10327, and is located on chromosome 1 with reported cytogenetic location 1p33-p32. The gene resides in genomic locus NT_{—}032972 (NCBI Genome Annotation). Aldehyde reductase (aldo-keto reductase family 1, member A1) reduces carbonyl-containing substrates, and may metabolize xenobiotics. It is a NADPH-dependent member of the aldo-keto reductase superfamily.

CPS 10 corresponds to ID2 which encodes inhibitor of DNA binding 2, dominant negative helix-loop-helix protein. This gene has LocusID: 3398, and is located on chromosome 2 with reported cytogenetic location 2p25. The gene resides in genomic locus NT_{—}005334 (NCBI Genome Annotation). The gene product is a member of the Id helix-loop-helix family of proteins, and may negatively regulate cell differentiation.

CPS 10 also has 95% sequence identity with an intron sequence of PTPRG. PTPRG encodes protein tyrosine phosphatase, receptor type, G, and has LocusID: 5793 with reported cytogenetic location 3p21-p14. The protein encoded by PTPRG gene is a member of the protein tyrosine phosphatase (PTP) family. PTPs are known to be signaling molecules that regulate a variety of cellular processes including cell growth, differentiation, mitotic cycle, and oncogenic transformation. The PTP encoded by PTPRG gene possesses an extracellular region, a single transmembrane region, and two tandem intracytoplasmic catalytic domains, and thus represents a receptor-type PTP. The extracellular region of this PTP contains a carbonic anhydrase-like (CAH) domain, which is also found in the extracellular region of PTPRBETA/ZETA. PTPRG gene is located in a chromosomal region that is frequently deleted in renal cell carcinoma and lung carcinoma, and thus is thought to be a candidate tumor suppressor gene.

In addition, CPS 10 aligns with a chromosomal region near LOC140282 with 88% sequence identity. LOC140282 encodes a protein similar to translationally controlled tumor protein (TCTP) (p23) (Histamine-releasing factor) (HRF), and has reported cytogenetic location 21 q21.3.

Affymetrix annotation suggests that CPS 11 corresponds to PSG7 which encodes pregnancy specific beta-1-glycoprotein 7 and has LocusID: 5676. PSG7 is located at chromosome 19q13.2.

Blast search of the Entrez human genome sequence database shows that CPS 11 aligns with a region 3′ to PSG1 with at least 98% sequence identity. PSG1 encodes pregnancy specific beta-1-glycoprotein, and has LocusID: 5669 with reported cytogenetic location 19q13.2. The gene product is a member of the pregnancy-specific glycoprotein (PSG) and CEA families.

Moreover, CPS 11 has 92-95% sequence identity with various regions on chromosome 19. They include PSG2, PSG9, a region near PSG3, and a region near PSG7. PSG2 encodes pregnancy specific beta-1-glycoprotein 2, and has LocusID: 5670 with reported cytogenetic location 19q13.1-q13.2. PSG9 encodes pregnancy specific beta-1-glycoprotein 9, and has LocusID: 5678 with reported cytogenetic location 19q13.2. PSG3 encodes pregnancy specific beta-1-glycoprotein 3, and has LocusID: 5671 with reported cytogenetic location 19q13.2. PSG7 encodes pregnancy specific beta-1-glycoprotein 7, and has LocusID: 5676 with reported cytogenetic location 19q13.2.

CPS 13 corresponds to IDH2 which encodes isocitrate dehydrogenase 2 (NADP+), mitochondrial. This gene has LocusID: 3418, and is located on chromosome 15 with reported cytogenetic location 15q26.1. The gene resides in genomic locus NT_{—}033276 (NCBI Genome Annotation). Mitochondrial NADP(+)-specific isocitrate dehydrogenase 2 can decarboxylate isocitrate into alpha-ketoglutarate.

CPS 14 corresponds to RER1 which encodes a protein similar to S. cerevisiae RER1. This gene has LocusID: 11079, and is located on chromosome 1 with reported cytogenetic location 1pter-q24. The gene resides in genomic locus NT_{—}004350 (NCBI Genome Annotation).

CPS 15 corresponds to TUBB which encodes tubulin, beta polypeptide. This gene has LocusID: 7280, and is located on chromosome 6 with reported cytogenetic location 6p21.3. The gene resides in genomic locus NT_{—}034880 (NCBI Genome Annotation). Beta-tubulin polymerizes to form microtubules, and is a member of a family of structural proteins.

CPS 16 corresponds to HADH2 which encodes hydroxyacyl-Coenzyme A dehydrogenase, type II. This gene has LocusID: 3028, and is located on chromosome X with reported cytogenetic location Xp 11.2. The gene resides in genomic locus NT_{—}011799 (NCBI Genome Annotation). The gene product can bind to amyloid-beta peptide.

CPS 17 corresponds to NDUFB5 which encodes NADH dehydrogenase (ubiquinone) 1 beta subcomplex, 5 (16 kD, SGDH). This gene has LocusID: 4711, and is located on chromosome 3 with reported cytogenetic location 3q27.1. The gene resides in genomic locus NT_{—}022396 (NCBI Genome Annotation). The gene product is a subunit of NADH-ubiquinone oxidoreductase (complex I). It can transport electrons from NADH to ubiquinone.

CPS 17 also shows 77% sequence identity with a chromosomal region near LOC205833. LOC205833 has reported cytogenetic location 4q34.3.

CPS 18 corresponds to APRT which encodes adenine phosphoribosyltransferase. This gene has LocusID: 353, and is located on chromosome 16 with reported cytogenetic location 16q24. The gene resides in genomic locus NT_{—}010404 (NCBI Genome Annotation). Adenine phosphoribosyltransferase belongs to the purine/pyrimidine phosphoribosyltransferase family. This enzyme catalyzes the formation of AMP and inorganic pyrophosphate from adenine and 5-phosphoribosyl-1-pyrophosphate (PRPP). It can also produce adenine as a by-product of the polyamine biosynthesis pathway. A homozygous deficiency in this enzyme may cause 2,8-dihydroxyadenine urolithiasis.

CPS 19 corresponds to PSME2 which encodes proteasome (prosome, macropain) activator subunit 2 (PA28 beta). This gene has LocusID: 5721, and is located on chromosome 14 with reported cytogenetic location 14q11.2. The gene resides in genomic locus NT_{—}025892 (NCBI Genome Annotation).

CPS 19 also aligns with LOC220462 with 97% sequence identity. LOC220462 encodes a protein similar to proteasome activator complex subunit 2 (Proteasome activator 28-beta subunit) (PA28beta) (PA28b) (Activator of multicatalytic protease subunit 2) (11S regulator complex beta subunit) (REG-beta). LOC220462 has reported cytogenetic location 13q14.11. In addition, CPS 19 has about 90-96% sequence identity with LOC257093, LOC 166868, and an intron sequence of LOC152940. LOC257093 encodes a protein similar to proteasome activator complex subunit 2 (Proteasome activator 28-beta subunit), and is located on chromosome 5. LOC 166868 encodes a protein similar to PA28beta, and has reported cytogenetic location 4 p14. LOC152940 has reported cytogenetic location 4q31.3-q32.1. Furthermore, fragments of CPS 17 show 86-97% sequence identity with LOC220220 and LOC206704. LOC220220 encodes a protein similar to PA28beta, and has reported cytogenetic location 10p11.23. LOC206704 also encodes a protein similar to PA28beta, and is located on chromosome 8p21.1.

CPS 20 corresponds to MTCP1 which encodes mature T-cell proliferation 1. This gene has LocusID: 4515, and is located on chromosome X with reported cytogenetic location Xq28. The gene resides in genomic locus NT_{—}025965 (NCBI Genome Annotation). The gene product may be involved in the leukemogenic process of mature T cell proliferation.

CPS 21 corresponds to NDUFB3 which encodes NADH dehydrogenase (ubiquinone) 1 beta subcomplex, 3 (12 kD, B12). This gene has LocusID: 4709, and is located on chromosome 2 with reported cytogenetic location 2q31.3. The gene resides in genomic locus NT_{—}005370 (NCBI Genome Annotation). The multisubunit NADH:ubiquinone oxidoreductase (complex I) is an enzyme complex in the electron transport chain of mitochondria. NDUFB3 gene product is a subunit of NADH-ubiquinone oxidoreductase (complex I), and can transport electrons from NADH to ubiquinone.

CPS 21 also has about 96% sequence identity with NDUFB3P4. NDUFB3P4 encodes NADH dehydrogenase (ubiquinone) 1 beta subcomplex, 3 (12 kD, B12) pseudogene 4. It has LocusID: 93995, and is located on chromosome 14. The gene is located in an intron of WARS which encodes tryptophanyl-tRNA synthetase and has LocusID: 7453 with reported cytogenetic location 14q32.31.

Moreover, CPS 21 has about 86-91% sequence identity with NDUFB3P3, and intron sequence of KIAA0893, and an intron sequence of PPP6C. NDUFB3P3 encodes NADH dehydrogenase (ubiquinone) 1 beta subcomplex, 3 (12 kD, B12) pseudogene 3. It has LocusID: 93996, and is located on chromosome 14. KIAA0893 encodes KIAA0893 protein, and has LocusID: 22911 with reported cytogenetic location 1p13.2. PPP6C encodes protein phosphatase 6, catalytic subunit, and has LocusID: 5537 with reported cytogenetic location 9q34.11.

CPS 23 corresponds to CTSL which encodes cathepsin L. This gene has LocusID: 1514, and is located on chromosome 9 with reported cytogenetic location 9q21-q22. The gene resides in genomic locus NT_{—}023935 (NCBI Genome Annotation). The protein encoded by CTSL gene is a lysosomal cysteine proteinase that plays a role in intracellular protein catabolism. Its substrates include collagen and elastin, as well as alpha-1 protease inhibitor, a major controlling element of neutrophil elastase activity. The encoded protein has been implicated in several pathologic processes, including myofibril necrosis in myopathies and in myocardial ischemia, and in the renal tubular response to proteinuria. This protein is a member of the peptidase C1 family. At least two transcript variants encoding the same protein have been found for CTSL gene.

Fragments of CPS 23 have 84-88% sequence identity with LOC118945, LOC119215, and LOC219343. LOC118945, LOC119215, and LOC219343 encode proteins similar to cathepsin L precursor (Major excreted protein) (MEP), and are located at chromosome 10q23.32, 10q21.1 and 10q23.2, respectively.

Affymetrix annotation suggests that CPS 24 corresponds to FGL2 which encodes fibrinogen-like 2 and has LocusID: 10875. FGL2 has the reported cytogenetic location at chromosome 7q11.23.

Blast search of the Entrez human genome database shows that CPS 24 aligns with the non-protein-coding strand of KIAA1505 with at least 98% sequence identity. KIAA1505 encodes KIAA1505 protein, and has LocusID: 57639 with reported cytogenetic location 7 p12.3. KIAA1505 resides in genomic locus NT_{—}007933 (NCBI Genome Annotation).

Affymetrix annotation suggests that CPS 25 corresponds to DKFZP564C1940 which is also known as LRP10. LRP10 encodes low density lipoprotein receptor-related protein 10, and has LocusID: 26020. LRP10 is located at chromosome 14q11.1.

Blast search of the Entrez human genome sequence database shows that fragments of CPS 26 have 84-98% sequence identity with various regions on chromosomes 1, 2, 4, 5, 7, 9, 10, 11, 12, 17, 18, X, or Y. For instance, nucleotides 164-283 and 359-589 have 95% sequence identity with an intron sequence of LOC121292. LOC121292 is located at chromosome 12q24.31, and resides in genomic locus NT_{—}035241 (NCBI Genome Annotation). Nucleotides 253-323 and 359-589 of CPS 26 align with a region near LOC138924 with at least 95% sequence identity. LOC138924 encodes a protein similar to peptidyl-Pro cis trans isomerase, and is located at chromosome 9q21.31 and in genomic locus NT_{—}008580 (NCBI Genome Annotation). Nucleotides 374-588 of CPS 26 have 93% sequence identity with a region between DIM1 and FLJ21172. DIMI encodes a protein similar to S. pombe dim1+, and has LocusID: 10907 with reported cytogenetic location 18q23. FLJ21172 encodes hypothetical protein FLJ21172, and has LocusID: 79863 with reported cytogenetic location 18q23. Nucleotides 634-1116 of CPS 26 have 86% sequence identity with an intron sequence of MYO1D. MYO1D encodes myosin ID, and has LocusID: 4642 and reported cytogenetic location 17q11-q12.

CPS 27 corresponds to SEMA3F which encodes sema domain, immunoglobulin domain (Ig), short basic domain, secreted, (semaphorin) 3F. This gene has LocusID: 6405, and is located on chromosome 3 with reported cytogenetic location 3p21.3. The gene resides in genomic locus NT_{—}006014 (NCBI Genome Annotation). The semaphorins are a family of proteins that are involved in signaling. A typical family member has a secretion signal, a 500-amino acid sema domain, and 16 conserved cysteine residues. Sequence comparisons have grouped the secreted semaphorins into 3 general classes. Members of the semaphorin III family, including human semaphorin III, chicken collapsin, and mouse semaphorins A, D, and E, have a basic domain at the C terminus. SEMA3F gene product is a secreted member of the semaphorin III family.

CPS 28 corresponds to BST2 which encodes bone marrow stromal cell antigen 2. This gene has LocusID: 684, and is located on chromosome 19 with reported cytogenetic location 19 p13.2. The gene resides in genomic locus NT_{—}011295 (NCBI Genome Annotation). Bone marrow stromal cells are involved in the growth and development of B-cells. Bone marrow stromal cell antigen 2 may play a role in pre-B-cell growth and in rheumatoid arthritis.

Blast search of the Entrez human genome sequence database shows that CPS 29 overlaps or includes a chromosomal region between SLC7A1 and KIAA0774. SLC7A1 encodes solute carrier family 7 (cationic amino acid transporter, y+system), member 1. SLC7A1 has LocusID: 6541, and is located on chromosome 13 with reported cytogenetic location 13q12-q14. KIAA0774 encodes KIAA0774 protein, and has LocusID: 23281. KIAA0774 is located on chromosome 13 with reported cytogenetic location 13q12.2. Both SLC7A1 and KIAA0774 have genomic locus NT_{—}009799 (NCBI Genome Annotation). CPS 29 is located 3′ to the protein-coding regions of both genes. CPS 29 matches with the protein-coding strand of SLC7A1. SLC7A1 gene product can has strong similarity to murine Rec-1 (Atrc1), and can transport arginine, lysine and ornithine across the plasma membrane.

CPS 30 corresponds to UBE2L6 which encodes ubiquitin-conjugating enzyme E2L 6. This gene has LocusID: 9246, and is located on chromosome 11 with reported cytogenetic location 11q12. The gene resides in genomic locus NT_{—}033903 (NCBI Genome Annotation). The gene product is a member of the ubiquitin-conjugating enzyme family, and can ubiquitinate cellular proteins and mark them for degradation. The gene product can also bind to Hect domains of E3 proteins.

CPS 31 corresponds to SLC 11A1 which encodes solute carrier family 11 (proton-coupled divalent metal ion transporters), member 1. This gene has LocusID: 6556, and is located on chromosome 2 with reported cytogenetic location 2q35. The gene resides in genomic locus NT_{—}005403 (NCBI Genome Annotation). The gene product is similar to murine Bcg (Nramp1), and may control antimicrobial activity of macrophages.

CPS 32 corresponds to CDKN1C which encodes cyclin-dependent kinase inhibitor 1C (p57, Kip2). This gene has LocusID: 1028, and is located on chromosome 11 with reported cytogenetic location 11p15.5. The gene resides in genomic locus NT_{—}009368 (NCBI Genome Annotation). Cyclin-dependent kinase inhibitor 1C is a tight-binding inhibitor of several G1 cyclin/Cdk complexes and a negative regulator of cell proliferation. Mutations of CDKN1C are implicated in sporadic cancers and Beckwith-Wiedemann syndrome, suggesting that CDKN1C may be a tumor suppressor candidate.

CPS 32 also has 98% sequence identity with a chromosomal region near LOC256784. LOC256784 encodes a protein similar to cyclin-dependent kinase inhibitor 1C (Cyclin-dependent kinase inhibitor P57) (P57KIP2), and is located on chromosome 11 with genomic locus NT_{—}009368 (NCBI Genome Annotation).

CPS 33 corresponds to GAIP (RGS19) which encodes regulator of G-protein signaling 19. This gene has LocusID: 10287, and is located on chromosome 20 with reported cytogenetic location 20q13.3. The gene resides in genomic locus NT_{—}011333 (NCBI Genome Annotation). G proteins mediate a number of cellular processes. The protein encoded by this gene belongs to the RGS (regulators of G-protein signaling) family and can interact with G protein, GAI3. G-protein signaling 19 is a guanosine triphosphatase-activating protein that may function to down-regulate Galpha i/Galpha q-linked signaling.

CPS 34 corresponds to TACC2 which encodes transforming, acidic coiled-coil containing protein 2. This gene has LocusID: 10579, and is located on chromosome 10 with reported cytogenetic location 10q26. The gene resides in genomic locus NT_{—}030764 (NCBI Genome Annotation). Transforming acidic coiled-coil proteins are a conserved family of centrosome- and microtubule-interacting proteins that are implicated in cancer. The member encoded by TACC2 gene concentrates at centrosomes throughout the cell cycle, and it is a candidate breast tumor suppressor and biomarker for tumor progression.

CPS 35 corresponds to CLNS1A which encodes chloride channel, nucleotide-sensitive, 1A. This gene has LocusID: 1207, and is located on chromosome 11 with reported cytogenetic location 11q13.5-q14. The gene resides in genomic locus NT_{—}033927 (NCBI Genome Annotation). The gene product is associated with a swelling-induced chloride channel, and may be involved in aqueous humor formation in the eye

CPS 35 also has 88% sequence identity with an intron sequence of TDRKH. TDRKH encodes tudor and KH domain-containing protein, and has LocusID: 11022. It is located at chromosome 1q21. In addition, nucleotides 66-459 of CPS 35 have 89% sequence identity with a region near LOC221349. LOC221349 encodes a protein similar to chloride conductance regulatory protein ICln (I(Cln)) (Chloride channel, nucleotide sensitive 1A) (Chloride ion current inducer protein) (ClCI) (Reticulocyte PICln), and has reported cytogenetic location 6p11.2. Nucleotides 1-66 of CPS 35 aligns with LOC152922 with 98% sequence identity. LOC152922 encodes a protein similar to chloride conductance regulatory protein ICln (I(Cln)) (Chloride channel, nucleotide sensitive 1A) (Chloride ion current inducer protein) (ClCI) (Reticulocyte PICln), and is located at chromosome 4q32.3.

CPS 36 corresponds to DPYSL2 which encodes dihydropyrimidinase-like 2. This gene has LocusID: 1808, and is located on chromosome 8 with reported cytogenetic location 8p22-p21. The gene resides in genomic locus NT_{—}023666 (NCBI Genome Annotation). The gene product is a member of the dihydropyrimidinase family.

CPS 37 corresponds to FCGR3A which encodes Fc fragment of IgG, low affinity IIIa, receptor for (CD16). This gene has LocusID: 2214, and is located on chromosome 1 with reported cytogenetic location 1q23. The gene resides in genomic locus NT_{—}004668 (NCBI Genome Annotation). The gene product is type III Fc gamma receptor, and can associate with zeta chain of the T-cell receptor complex (CD3Z). The gene product is a member of the immunoglobulin superfamily

CPS 38 corresponds to UQCRC1 which encodes ubiquinol-cytochrome c reductase core protein I. This gene has LocusID: 7384, and is located on chromosome 3 with reported cytogenetic location 3p21.3. The gene resides in genomic locus NT_{—}005990 (NCBI Genome Annotation). The gene product, core I protein, is a subunit of the ubiquinol-cytochrome c oxidoreductase in the mitochondrial respiratory chain.

CPS 39 corresponds to GZMA which encodes granzyme A (granzyme 1, cytotoxic T-lymphocyte-associated serine esterase 3). This gene has LocusID: 3001, and is located on chromosome 5 with reported cytogenetic location Sq11-q12. The gene resides in genomic locus NT_{—}006431 (NCBI Genome Annotation). Cytolytic T lymphocytes (CTL) and natural killer (NK) cells share the ability to recognize, bind, and lyse specific target cells. They are thought to protect their host by lysing cells bearing on their surface “nonself” antigens, usually peptides or proteins resulting from infection by intracellular pathogens. GZMA gene product is a T cell- and natural killer cell-specific serine protease that may function as a common component necessary for lysis of target cells by cytotoxic T lymphocytes and natural killer cells.

CPS 41 corresponds to PSMB10 which encodes proteasome (prosome, macropain) subunit, beta type, 10. This gene has LocusID: 5699, and is located on chromosome 16 with reported cytogenetic location 16q22.1. The gene resides in genomic locus NT_{—}010478 (NCBI Genome Annotation). The gene can replace beta subunit PSMB7 when cells are stimulated by interferon g.

CPS 42 corresponds to IDS which encodes iduronate 2-sulfatase (Hunter syndrome). This gene has LocusID: 3423, and is located on chromosome X with reported cytogenetic location Xq28. The gene resides in genomic locus NT_{—}019686 (NCBI Genome Annotation). Iduronate-2-sulfatase is involved in the lysosomal degradation of heparan sulfate and dermatan sulfate. Mutations in this X-chromosome gene that result in enzymatic deficiency may lead to the sex-linked Mucopolysaccharidosis Type II, also known as Hunter Syndrome. Iduronate-2-sulfatase has a sequence homology with human arylsulfatases A, B, and C, and human glucosamine-6-sulfatase. A splice variant of this gene has been described.

Affymetrix annotation suggests that CPS 43 corresponds to AHNAK which encodes AHNAK nucleoprotein (desmoyokin) and has LocusID: 195. The gene is located at chromosome 11 q12-q 13.

Blast search of the Entrez human genome sequence database indicates that CPS 43 aligns with a region 3′ to LOC221087 with 100% sequence identity. LOC221087 is hypothetical gene supported by M80899. The gene is located on chromosome 11q13.1, and resides in genomic locus NT_{—}033241 (NCBI Genome Annotation).

CPS 44 corresponds to PTPN7 which encodes protein tyrosine phosphatase, non-receptor type 7. This gene has LocusID: 5778, and is located on chromosome 1 with reported cytogenetic location 1q32.1. The gene resides in genomic locus NT_{—}034408 NCBI Genome Annotation). The gene product is a member of the protein tyrosine phosphatase (PTP) family. PTPs are known to be signaling molecules that regulate a variety of cellular processes including cell growth, differentiation, mitotic cycle, and oncogenic transformation. PTPN7 gene is expressed in a variety of hematopoietic cells, and is an early response gene in lymphokine stimulated cells. The noncatalytic N-terminus of PTPN7 gene product can interact with MAP kinases and suppress the MAP kinase activities. The gene product is also shown to be involved in the regulation of T cell antigen receptor (TCR) signaling, which is thought to function through dephosphorylating the molecules related to MAP kinase pathway. At least three alternatively spliced transcript variants of this gene, which encode at least two distinct isoforms, have been reported.

CPS 45 corresponds to DUSP2 which encodes dual specificity phosphatase 2. This gene has LocusID: 1844, and is located on chromosome 2 with reported cytogenetic location 2q11. The gene resides in genomic locus NT_{—}026970 (NCBI Genome Annotation). The protein encoded by DUSP2 is a member of the dual specificity protein phosphatase subfamily. These phosphatases can inactivate their target kinases by dephosphorylating both the phosphoserine/threonine and phosphotyrosine residues. They negatively regulate members of the mitogen-activated protein (MAP) kinase superfamily (MAPK/ERK, SAPK/JNK, p38), which are associated with cellular proliferation and differentiation. Different members of the family of dual specificity phosphatases show distinct substrate specificities for various MAP kinases, different tissue distribution and subcellular localization, and different modes of inducibility of their expression by extracellular stimuli. DUSP2 gene product can inactivate ERK1 and ERK2, is expressed in hematopoietic tissues, and is localized in the nucleus.

Affymetrix annotation suggests that CPS 46 corresponds to PSMB9 which encodes proteasome (prosome, macropain) subunit, beta type, 9 (large multifunctional protease 2). PSMB9 has LocusID: 5698, and is located at chromosome 6p21.3.

Blast search of the Entrez human genome sequence database indicates that CPS 46 shows 100% sequence identity with three regions near PSMB8. These regions are within genomic locus NT_{—}007592 (NCBI Genome Annotation). PSMB8 encodes proteasome (prosome, macropain) subunit, beta type, 8 (large multifunctional protease 7). It has LocusID: 5696 and reported cytogenetic location 6p21.3.

Blast search of the Entrez human genome sequence database shows that CPS 47 overlaps or includes an intron sequence of HAN11. HAN11 encodes WD-repeat protein, and has LocusID: 10238 with reported cytogenetic location 17q21.33. HAN11 resides in genomic locus NT_{—}035428 (NCBI Genome Annotation). HAN11 gene product has WD-repeats, and is similar to plant an11.

Blast search of the Entrez human genome sequence database also shows that CPS 48 overlaps or includes LILRB1. LILRB1 encodes leukocyte immunoglobulin-like receptor, subfamily B (with TM and ITIM domains), member 1. LILRB1 has LocusID: 10859, and is located on chromosome 19 with reported cytogenetic location 19q13.4. LILRB1 resides in genomic locus NT_{—}011225 (NCBI Genome Annotation). The gene product (leukocyte immunoglobulin-like receptor B1) contains immunoreceptor tyrosine-based inhibitory motifs, and can bind to cellular and viral MHC class I antigens.

CPS 48 also aligns with LILRB2 with 91% sequence identity. LILRB2 encodes leukocyte immunoglobulin-like receptor, subfamily B (with TM and ITIM domains), member 2. The gene has LocusID: 10288 and reported cytogenetic location 19q13.4.

CPS 49 corresponds to SDR1 which encodes short-chain dehydrogenase/reductase 1. This gene has LocusID: 9249, and is located on chromosome 1 with reported cytogenetic location 1p36.1. The gene resides in genomic locus NT_{—}022041 (NCBI Genome Annotation). Short-chain dehydrogenase/reductase 1 can reduce all-trans-retinal during bleached visual pigment regeneration.

Affymetrix annotation suggests that CPS 50 corresponds to POLR2L which encodes polymerase (RNA) II (DNA directed) polypeptide L, 7.6 kDa. POLR2L has LocusID: 5441, and is located at chromosome 11p15.

CPS 51 corresponds to SCYA2 which encodes small inducible cytokine A2 (monocyte chemotactic protein 1). This gene has LocusID: 6347, and is located on chromosome 17 with reported cytogenetic location 17q11.2-q21.1. The gene resides in genomic locus NT_{—}010799 (NCBI Genome Annotation). Cytokine A 2 is a chemotactic factor for monocytes.

CPS 52 corresponds to PAI2 (SERPINB2) which encodes serine (or cysteine) proteinase inhibitor, clade B (ovalbumin), member 2. This gene has LocusID: 5055, and is located on chromosome 18 with reported cytogenetic location 18q21.3. The gene resides in genomic locus NT_{—}033907 (NCBI Genome Annotation). The gene product is also known as plasminogen activator inhibitor II, and may function as a serine protease inhibitor. It is considered a member of the serpin family of serine protease inhibitors.

Blast search of the Entrez human genome sequence database shows that CPS 53 overlaps or includes LOC158972. LOC158972 has reported cytogenetic location Xq28, and is located in genomic locus NT_{—}026504 (NCBI Genome Annotation).

CPS 53 also aligns with a chromosomal region near LOC93052 with 93% sequence identity. LOC93052 has reported cytogenetic location Xq28.

Blast search of the Entrez human genome sequence database shows that CPS 54 overlaps or includes KIAA1564. KIAA1564 encodes KIAA1564 protein, and has LocusID: 57680 with reported cytogenetic location 14q11.1. KIAA1564 resides in genomic locus NT_{—}025892 (NCBI Genome Annotation). CPS 54 matches with the non-protein-coding strand of KIAA1564.

Affymetrix annotation indicates that CPS 55 corresponds to JMJ. Nucleotides 1997 to 198161 of AL021938 (SEQ ID NO:52) have 99% sequence identity with JMJ. JMJ encodes jumonji homolog (mouse), and has LocusID: 3720. The gene is located on chromosome 6 with reported cytogenetic location 6p24-p23 and genomic locus NT_{—}007592 (NCBI Genome Annotation). JMJ is an ortholog of the mouse jumonji gene, which encodes a nuclear protein involved in mouse embryogenesis, including neural tube formation. Overexpression of mouse jumonji negatively regulates cell proliferation. The jumonji proteins contain a DNA-binding domain, called an AT-rich interaction domain (ARID), and share regions of similarity with human retinoblastoma-binding protein-2 and the human SMCX protein. Nucleotides 1997 to 198161 of AL021938 match with the non-protein-coding strand of JMJ.

Nucleotides 1 to 66 of CPS 55 have 100% sequence identity with a chromosomal region in genomic locus NT_{—}010463 (NCBI Genome Annotation). This chromosomal region is located 3′ to LOC222499 which has reported cytogenetic location 16q21.

CPS 56 corresponds to UGP2 which encodes UDP-glucose pyrophosphorylase 2. This gene has LocusID: 7360, and is located on chromosome 2 with reported cytogenetic location 2p14-p13. The gene resides in genomic locus NT_{—}005375 (NCBI Genome Annotation). The enzyme encoded by UGP2 is an intermediary in mammalian carbohydrate interconversions. It can transfer a glucose moiety from glucose-1-phosphate to MgUTP and form UDP-glucose and MgPPi.

Nucleotides 155 to 433 of CPS 56 have 0.96% sequence identity with LOC253415. LOC253415 encodes a protein similar to UDP-glucose pyrophosphorylase 2 (UTP-glucose-1-phosphate uridyltransferase) (UDP-glucose diphosphorylase) (UGPase 2). LOC253415 is located on chromosome 2.

CPS 57 corresponds to KIAA0410. This gene has LocusID: 9818, and is located on chromosome 13 with reported cytogenetic location 13q12.12. The gene resides in genomic locus NT_{—}009799 (NCBI Genome Annotation).

CPS 58 corresponds to NDUFS7 which encodes NADH dehydrogenase (ubiquinone) Fe-S protein 7 (20 kD) (NADH-coenzyme Q reductase). This gene has LocusID: 4727, and is located on chromosome 19 with reported cytogenetic location 19p13. The gene resides in genomic locus NT_{—}011268 (NCBI Genome Annotation).

CPS 59 corresponds to KIAA0645. This gene has LocusID: 9681, and is located on chromosome 22 with reported cytogenetic location 22q12.3. The gene resides in genomic locus NT_{—}011520 (NCBI Genome Annotation).

CPS 60 corresponds to GSTP1 which encodes glutathione S-transferase pi. This gene has LocusID: 2950, and is located on chromosome 11 with reported cytogenetic location 11q13. The gene resides in genomic locus NT_{—}033241 (NCBI Genome Annotation). Glutathione S-transferases (GSTs) are a family of enzymes that play a role in detoxification by catalyzing the conjugation of many hydrophobic and electrophilic compounds with reduced glutathione. The soluble GSTs are categorized into 4 main classes: alpha, mu, pi, and theta. The glutathione S-transferase pi gene (GSTP1) is a polymorphic gene encoding active, functionally different GSTP1 variant proteins that are thought to function in xenobiotic metabolism and play a role in susceptibility to cancer, and other diseases.

Nucleotides 180 to 558 have 86% sequence identity with a chromosomal region near DGKA. DGK encodes diacylglycerol kinase, alpha (80 kD), and is located at chromosome 12q13.3 with LocusID: 1606.

CPS 61 corresponds to DECR1 which encodes 2,4-dienoyl CoA reductase 1, mitochondrial. This gene has LocusID: 1666, and is located on chromosome 8 with reported cytogenetic location 8q21.3. The gene resides in genomic locus NT_{—}034898 (NCBI Genome Annotation). The gene product is an accessory enzyme which participates in the beta-oxidation and metabolism of unsaturated fatty enoyl-CoA esters.

CPS 62 corresponds to PLXNC1 which encodes plexin C1. This gene has LocusID: 10154, and is located on chromosome 12 with reported cytogenetic location 12q23.3. The gene resides in genomic locus NT_{—}009575 (NCBI Genome Annotation). Plexin C1 can function as a receptor for virally-encoded semphorin. It is a member of the plexin family.

CPS 63 corresponds to TUBA2 which encodes tubulin, alpha 2. This gene has LocusID: 7278, and is located on chromosome 13 with reported cytogenetic location 13q11. The gene resides in genomic locus NT_{—}009799 (NCBI Genome Annotation). Microtubules of the eukaryotic cytoskeleton perform essential and diverse functions and are composed of a heterodimer of alpha and beta tubulin. The genes encoding these microtubule constituents are part of the tubulin superfamily, which is composed of six distinct families. Genes from the alpha, beta and gamma tubulin families are found in all eukaryotes. The alpha and beta tubulins represent the major components of microtubules, while gamma tubulin plays a critical role in the nucleation of microtubule assembly. There are multiple alpha and beta tubulin genes and they are conserved among and between species. TUBA2 gene is an alpha tubulin gene that encodes a protein similar to the mouse testis-specific Tuba3 and Tuba7 gene products. TUBA2 gene is located in the 13q11 region, which is associated with the genetic diseases Clouston hidrotic ectodermal dysplasia and Kabuki syndrome. Alternative splicing has been observed for this gene and at least two variants have been identified.

CPS 63 has 95-96% sequence identity with H2-ALPHA and LOC112714. H2-ALPHA encodes alpha-tubulin isotype H2-alpha, and has LocusID: 113457 with reported cytogenetic location 2q22.1. LOC112714 encodes a protein similar to alpha tubulin, and has LocusID: 112714 with reported cytogenetic location 2q14.2.

In addition, CPS 63 shows 85-90% sequence identity with a chromosomal region near MGC16703. MGC16703 encodes alpha tubulin-like, and is located at chromosome 22q11.21 with LocusID: 113691. Fragments of CPS have 83-91% sequence identity with regions on chromosomes 1 and 22.

CPS 64 corresponds to NME1 which encodes non-metastatic cells 1, protein (NM23A) expressed in. This gene has LocusID: 4830, and is located on chromosome 17 with reported cytogenetic location 17q21.3. The gene resides in genomic locus NT_{—}010783 (NCBI Genome Annotation). NME1 was reported to have reduced mRNA transcript levels in highly metastatic cells. NME1 encodes the “A” isoform of nucleoside diphosphate kinase (NDK). NDK exists as a hexamer composed of the “A” (encoded by NME1) and “B” (encoded by NME2) isoforms. Mutations in NME1 have been identified in aggressive neuroblastomas. NME1 gene product may have a role in the transcriptional regulation of c-myc expression.

Blast search of the Entrez human genome sequence database shows that CPS 65 overlaps or includes a chromosomal region between SLC11A1 and NLI-IF. Both genes are located on chromosome 2 with reported cytogenetic location 2q35 and genomic locus NT_{—}005403 (NCBI Genome Annotation). SLC11A1 encodes solute carrier family 11 (proton-coupled divalent metal ion transporters), member 1, and has LocusID: 6556. SLC11A1 gene product is similar to murine Bcg (Nramp1), and may control antimicrobial activity of macrophages. NLI-IF encodes nuclear LIM interactor-interacting factor, and has LocusID: 58190. NLI-IF gene product is similar to a region of S. cerevisiae plasma membrane phosphatase Psr2p. CPS 65 is located 3′ to the protein-coding sequence of SLC11A1 and 5′ to the protein-coding sequence of NL1-IF.

Affymetrix annotation suggests that CPS 66 corresponds to RNAHP which encodes RNA helicase-related protein and has LocusID: 11325. The gene has reported cytogenetic location at chromosome 17q22.

Blast search of the Entrez human genome sequence database shows that CPS 66 aligns with a chromosomal region at chromosome 16q13 with at least 95% sequence identity. This region resides in genomic locus NT_{—}010498 (NCBI Genome Annotation), and is located near MT1G. MT1G encodes metallothionein 1G, and has LocusID: 4495.

In addition, CPS 66 has 83-92% sequence identity with various regions on chromosomes 1, 4, 9, 16 and 20. These regions include MT1H, MT2P1, LOC255917, LOC127544, LOC149450, a region near MGC10731, and a region near MMP24. MT1H encodes metallothionein 1H, and has LocusID: 4496 with reported cytogenetic location 16q13. MT2P1 encodes metallothionein 2 pseudogene 1 (processed), and is located at chromosome 4p11-q21 (LocusID: 4503). MGC10731 has LocusID: 79363, and is located at chromosome 1p36.13. LOC255917 encodes a protein similar to Metallothionein-IE (MT-IE), and is located on chromosome 9. LOC127544 encodes a protein similar to dj1174N9.1 (novel protein with IBR domain), and is located at chromosome 1p34.3. LOC149450 has reported cytogenetic location 1q42.3. MMP24 encodes matrix metalloproteinase 24 (membrane-inserted), and is located at chromosome 20q11.2 with LocusID: 10893.

CPS 67 corresponds to ZNF198 which encodes zinc finger protein 198. This gene has LocusID: 7750, and is located on chromosome 13 with reported cytogenetic location 13q11-q12. The gene resides in genomic locus NT_{—}009799 (NCBI Genome Annotation). Zinc-finger protein 198 contains zinc fingers.

Nucleotides 185-221 of CPS 67 have 100% sequence identity with an intron sequence of LOC205936. LOC205936 is located at chromosome 4 p16.2, and resides in genomic locus NT_{—}006051 (NCBI Genome Annotation).

CPS 68 corresponds to PRCP which encodes prolylcarboxypeptidase (angiotensinase C). This gene has LocusID: 5547, and is located on chromosome 11 with reported cytogenetic location 11q14. The gene resides in genomic locus NT_{—}033927 (NCBI Genome Annotation). Prolylcarboxypeptidase (angiotensinase C) is a serine carboxypeptidase and can remove residues linked to proline.

CPS 70 corresponds to DKFZP586A0522 which encodes DKFZP586A0522 protein. This gene has LocusID: 25840, and is located on chromosome 12 with reported cytogenetic location 12q11. The gene resides in genomic locus NT_{—}009782 (NCBI Genome Annotation). The gene product include a region with low sequence similarity to a region of S. cerevisiae Coq5p. DKFZP586A0522 overlaps with LOC196529 which is encodes a protein similar to DKFZP586A0522 protein.

CPS 71 corresponds to SFRS11 which encodes splicing factor, arginine/serine-rich 11. This gene has LocusID: 9295, and is located on chromosome 1 with reported cytogenetic location 1p21-p34. The gene resides in genomic locus NT_{—}004464 (NCBI Genome Annotation). The gene product contains arginine/serine-rich domain and an RRM domain, and may have a role in pre-mRNA splicing.

Nucleotides 1 to 234 of CPS 71 have 89% sequence identity with an intron sequence of PEPP2. PEPP2 encodes phosphoinositol 3-phosphate-binding protein-2, and has LocusID: 54477 with reported cytogenetic location 12p12.

CPS 72 corresponds to RAC2 which encodes ras-related C3 botulinum toxin substrate 2 (rho family, small GTP binding protein Rac2). This gene has LocusID: 5880, and is located on chromosome 22 with reported cytogenetic location 22q13.1. The gene resides in genomic locus NT_{—}011520 (NCBI Genome Annotation). The protein encoded by RAC2 is a GTPase which belongs to the RAS superfamily of small GTP-binding proteins. Members of this superfamily appear to regulate a diverse array of cellular events, including the control of cell growth, cytoskeletal reorganization, and the activation of protein kinases. The RAC2 gene product may be a target for ADP ribosylation by the C3 subunit of botulinum toxin.

Nucleotides 400 to 487 of CPS 72 have 95% sequence identity with STK17A. STK17A encodes serine/threonine kinase 17a (apoptosis-inducing), and has LocusID: 9263 with reported cytogenetic location 7p12-p14.

CPS 73 corresponds to ZFP103 which encodes zinc finger protein 103 homolog (mouse). This gene has LocusID: 7844, and is located on chromosome 2 with reported cytogenetic location 2p11.2. The gene resides in genomic locus NT_{—}015805 NCBI Genome Annotation). The gene product contains a zinc-finger domain, and may be associated with membranous protein sorting.

CPS 74 corresponds to LOC51580 which encodes H-2K binding factor-2. This gene has LocusID: 51580, and is located on chromosome 9. The gene resides in genomic locus NT_{—}006316 (NCBI Genome Annotation). The gene product is a member of the recombination signal-sequence binding protein family. It is a transcription factor that binds to the NFkB site of MHC class I genes.

CPS 75 corresponds to NRD1 which encodes nardilysin (N-arginine dibasic convertase). This gene has LocusID: 4898, and is located on chromosome 1 with reported cytogenetic location 1p32.2-p32.1. The gene resides in genomic locus NT_{—}004424 (NCBI Genome Annotation). N-arginine dibasic convertase (NRD convertase) is a zinc-dependent endopeptidase. It is a member of the insulinase family.

Blast search of the Entrez human genome sequence database shows that CPS 76 overlaps or includes the 3′ untranslated region of FLJ20154. FLJ20154 encodes hypothetical protein FLJ20154, and is located at chromosome 10q24.32 with LocusID: 54838. FLJ20154 resides in genomic locus NT_{—}030059 (NCBI Genome Annotation).

CPS 76 also has 85-88% sequence identity with an intron sequence of KIAA0103, a chromosome 13 region near LOC160822, and a chromosome 7 region near BAP29. KIAA0103 has LocusID: 9694 and reported cytogenetic location 8q23.1. LOC160822 encodes a protein similar to hypothetical protein FLJ12457, and is located at chromosome 13q31.1. BAP29 encodes B-cell receptor-associated protein BAP29, and is located at chromosome 7q22.2 with LocusID: 55973. Moreover, fragments of CPS 76 align with various other regions on chromosome 1, 6, 15 and 19 with 89-90% sequence identity.

Affymetrix annotation suggests that CPS 77 corresponds to KIAA0906, also known as NUP210 which encodes nucleoporin 210. The gene has LocusID: 23225, and is located at chromosome 3p25.1.

Blast search of the Entrez human genome sequence database shows that CPS 77 aligns with FLJ22389 with at least 99% sequence identity. FLJ22389 has LocusID: 79985, and is located on chromosome 3 with reported cytogenetic location 3p25.1. The gene resides in genomic locus NT_{—}005927 (NCBI Genome Annotation).

Affymetrix annotation suggests that CPS 78 corresponds to MT1F which encodes metallothionein 1F (functional) and has LocusID: 4494. The gene is located at 16q13.

Blast search of the Entrez human genome sequence database shows that CPS 78 has 100% sequence identity with a chromosomal region located 3′ to the protein-coding sequence of MT1G. This chromosomal region, as well as MT1G, are within genomic locus NT_{—}010498 (NCBI Genome Annotation) on chromosome 16. MT1G encodes metallothionein 1G, and has LocusID: 4495 with reported cytogenetic location 16q13.

Nucleotides 1 to 67 of CPS 78 have 90-95% sequence identity to various regions in genomic locus NT_{—}010498. These regions include MT2A, LOC221228, MT1G and MT1L. MT2A encodes metallothionein 2A, and has LocusID: 4502 with reported cytogenetic location 16q13. LOC221228 is a hypothetical gene supported by AF495759. MT1G encodes metallothionein IG. MT1L encodes metallothionein IL, and has LocusID: 4500 with reported cytogenetic location 16q13.

In addition, CPS 78, and certain fragments thereof, have 89-93% sequence identity with MT2P1, LOC127544, LOC255917, a chromosomal region near MMP24, a chromosomal region near MGC 10731, and a chromosomal region near LOC 149450. MT2P1 encodes metallothionein 2 pseudogene 1 (processed), and has LocusID: 4503 with reported cytogenetic location 4p11-q21. LOC127544 encodes a protein similar to dJ1174N9.1 (novel protein with IBR domain), and is located at chromosome 1p34.3. LOC255917 encodes a protein similar to metallothionein-IE (MT-1E), and is located on chromosome 9. MMP24 encodes matrix metalloproteinase 24 (membrane-inserted), and is located at chromosome 20q11.2 with LocusID: 10893. MGC10731 encodes hypothetical protein MGC10731, and has LocusID: 79363 with reported cytogenetic location 1p36.13. LOC149450 is located at chromosome 1q42.3.

CPS 79 corresponds to P85SPR (ARHGEF7) which encodes Rho guanine nucleotide exchange factor (GEF) 7. This gene has LocusID: 8874, and is located on chromosome 13 with reported cytogenetic location 13q34. The gene resides in genomic locus NT_{—}009952 (NCBI Genome Annotation). Rho GTPases are involved in numerous cellular processes that are initiated by extracellular stimuli that work through G protein coupled receptors. The protein encoded by P85SPR belongs to a family of cytoplasmic proteins that activate the Ras-like family of Rho proteins by exchanging bound GDP for GTP. The encoded protein may form a complex with G proteins and stimulate Rho-dependent signals. The protein can induce membrane ruffling. The gene product is also involved in Pak recruitment to Cdc42- and Rac1-driven focal complexes. Multiple alternatively spliced transcript variants encoding different isoforms have been described for this gene.

CPS 80 corresponds to UNK_N53547 (MGC5508) which encodes hypothetical protein MGC5508. This gene has LocusID: 79073, and is located on chromosome 11 with reported cytogenetic location 11q13.1. The gene resides in genomic locus NT_{—}033903 (NCBI Genome Annotation).

CPS 81 corresponds to GPRK5 which encodes G protein-coupled receptor kinase 5. This gene has LocusID: 2869, and is located on chromosome 10 with reported cytogenetic location 10q24-qter. The gene resides in genomic locus NT_{—}008902 (NCBI Genome Annotation). G protein-coupled receptor kinases (GRKs) play a role in phosphorylating and regulating the activity of a variety of G protein-coupled receptors. G protein-coupled receptor kinase 5 can phosphorylate agonist-stimulated G protein-coupled receptors.

CPS 83 corresponds to SCYA7 which encodes small inducible cytokine A7 (monocyte chemotactic protein 3). This gene has LocusID: 6354, and is located on chromosome 17 with reported cytogenetic location 17q11.2-q12. The gene resides in genomic locus NT_{—}010799 (NCBI Genome Annotation). Monocyte chemotactic protein 3 is a secreted chemokine which attracts macrophages during inflammation and metastasis. It is a member of the C—C subfamily of chemokines which are characterized by having two adjacent cysteine residues. The protein is an in vivo substrate of matrix metalloproteinase 2, an enzyme which degrades components of the extracellular matrix. SCYA7 gene is part of a cluster of C—C chemokine family members on chromosome 17q.

CPS 84 corresponds to TUBA3 which encodes tubulin, alpha 3. This gene has LocusID: 7846, and is located on chromosome 12 with reported cytogenetic location 12q12-12q14.3. The gene resides in genomic locus NT_{—}009526 (NCBI Genome Annotation). There are multiple alpha and beta tubulin genes, which are conserved among species. TUBA3 encodes alpha tubulin and is similar to mouse and rat Tuba1 gene. Northern blotting studies have shown the gene expression in morphologically differentiated neurologic cells. TUBA3 is one of three alpha-tubulin genes in a cluster on chromosome 12q.

CPS 84 also has 97% sequence identity with a chromosomal region located 3′ to LOC 134262. LOC 134262 encodes a protein similar to alphaTub84B gene product. The gene is located at 5 μl 1, and resides in genomic locus NT_{—}023098.

CPS 85 corresponds to SCML2 which encodes sex comb on midleg-like 2 (Drosophila). This gene has LocusID: 10389, and is located on chromosome X with reported cytogenetic location Xp22. The gene resides in genomic locus NT_{—}011586 (NCBI Genome Annotation). The gene product is similar to Drosophila Scm.

CPS 85 also aligns with SCML1 with 94% sequence identity. SCML1 encodes sex comb on midleg-like 1 (Drosophila). The gene has LocusID: 6322, and is located on chromosome X with reported cytogenetic location Xp22.2-p22.1. The gene resides in genomic locus NT_{—}011586.

CPS 87 corresponds to ILIR1 which encodes interleukin 1 receptor, type I. This gene has LocusID: 3554, and is located on chromosome 2 with reported cytogenetic location 2q12. The gene resides in genomic locus NT_{—}022171 (NCBI Genome Annotation). Type I interleukin-1 receptor contains immmunoglobulin domains, and can bind to all three forms of interleukin-1 (IL1A, IL1B, and IL1RN).

CPS 88 corresponds to UNK_AL008729 (LOC221692) which encodes a protein similar to KIAA1733 protein. This gene is located on chromosome 6 with reported cytogenetic location 6p22.3. The gene resides in genomic locus NT_{—}007592 (NCBI Genome Annotation). LOC221692 is located within LOC51256 which has LocusID: 51256. CPS 88 matches with the non-protein-coding strand of LOC51256.

CPS 89 corresponds to KIAA0191 which encodes KIAA0191 protein. This gene has LocusID: 23318, and is located on chromosome 1 with reported cytogenetic location 1p32.3. The gene resides in genomic locus NT_{—}004424 (NCBI Genome Annotation).

CPS 90 corresponds to EGFL5 which encodes EGF-like-domain, multiple 5. This gene has LocusID: 1955, and is located on chromosome 9 with reported cytogenetic location 9q32-q33.3. The gene resides in genomic locus NT_{—}017568 (NCBI Genome Annotation).

CPS 91 corresponds to DUSPI which encodes dual specificity phosphatase 1. This gene has LocusID: 1843, and is located on chromosome 5 with reported cytogenetic location 5q34. The gene resides in genomic locus NT_{—}023132 (NCBI Genome Annotation). The expression of DUSPI gene can be induced in human skin fibroblasts by oxidative/heat stress and growth factors. The bacterially expressed and purified DUSPI protein has intrinsic phosphatase activity, and can inactivate mitogen-activated protein (MAP) kinase in vitro by the concomitant dephosphorylation of both its phosphothreonine and phosphotyrosine residues. DUSPI protein can also suppress the activation of MAP kinase by oncogenic ras in extracts of Xenopus oocytes. Thus, DUSPI may play a role in the human cellular response to environmental stress as well as in the negative regulation of cellular proliferation.

CPS 92 corresponds to FBP1 which encodes fructose-1,6-bisphosphatase 1. This gene has LocusID: 2203, and is located on chromosome 9 with reported cytogenetic location 9q22.3. The gene resides in genomic locus NT_{—}008476 (NCBI Genome Annotation). Fructose-1,6-bisphosphatase 1 is a gluconeogenesis regulatory enzyme. It can catalyze the hydrolysis of fructose 1,6-bisphosphate to fructose 6-phosphate and inorganic phosphate. Fructose-1,6-diphosphatase deficiency is associated with hypoglycemia and metabolic acidosis.

CPS 93 corresponds to HRB which encodes HIV-1 Rev binding protein. This gene has LocusID: 3267, and is located on chromosome 2 with reported cytogenetic location 2q36. The gene resides in genomic locus NT_{—}005403 (NCBI Genome Annotation). HIV-1 Rev binding protein can interact with the activation domains of the HIV-1 Rev protein, and may be related to nucleoporins, a class of proteins that mediate nucleocytoplasmic transport. HIV-1 Rev binding protein binds to the Rev activation domain when Rev is assembled onto its RNA target and can significantly enhance Rev activity when over-expressed. The HRB gene can expressed as a major 2.8-kb and a minor 4.6-kb mRNA.

CPS 94 corresponds to NAGA which encodes N-acetylgalactosaminidase, alpha-. This gene has LocusID: 4668, and is located on chromosome 22 with reported cytogenetic location 22q13-qter. The gene resides in genomic locus NT_{—}011520 (NCBI Genome Annotation). The lysosomal enzyme alpha-N-acetylgalactosaminidase can cleave alpha-N-acetylgalactosaminyl moieties from glycoconjugates. Mutations in NAGA have been implicated as the cause of Schindler disease types I and II (type II also known as Kanzaki disease).

CPS 95 corresponds to GLRX which encodes glutaredoxin (thioltransferase). This gene has LocusID: 2745, and is located on chromosome 5 with reported cytogenetic location 5q14. The gene resides in genomic locus NT_{—}023148 (NCBI Genome Annotation). Glutaredoxin can function as a glutathione-dependent hydrogen donor for ribonucleotide reductase.

Fragments of CPS 95 also align with AF358259, a chromosomal region near GABRA6 and a chromosomal region near GLRXP with 86-90% sequence identity. AF358259 encodes glutaredoxin pseudogene 2, and has LocusID: 171418 with reported cytogenetic location 14q32.13-q32.2. GABRA6 encodes gamma-aminobutyric acid (GABA) A receptor, alpha 6. It has LocusID: 2559, and is located at chromosome 5q34. GLRXP encodes glutaredoxin (thioltransferase) pseudogene, and is located at chromosome 20q11.2 with LocusID: 170522. In addition, nucleotides 1 to 29 of CPS 95 has 100% sequence identity with LOC257079. LOC257079 encodes a protein similar to glutaredoxin (Thioltransferase) (TTase), and is located on chromosome 5.

CPS 96 corresponds to UNK_W28281 (GABARAPL1) which encodes GABA(A) receptor-associated protein like 1. This gene has LocusID: 23710, and is located on chromosome 12 with reported cytogenetic location 12p13.1. The gene resides in genomic locus NT_{—}035207 (NCBI Genome Annotation).

SEQ ID NO: 360, which can be used as a probe sequence for detecting the expression level of UNK_W28281, also aligns with GABARAPL3 with about 95% sequence identity. GABARAPL3 encodes GABA(A) receptors associated protein like 3. It has LocusID: 23766, and is located on chromosome 15 with reported cytogenetic location 15q25.1.

CPS 97 corresponds to UNK_AL096740 (UBE3B) which encodes ubiquitin protein ligase. This gene has LocusID: 89910, and is located on chromosome. 12 with reported cytogenetic location 12q24.11. The gene resides in genomic locus NT_{—}009770 (NCBI Genome Annotation).

CPS 98 corresponds to TBXAS1 which encodes thromboxane A synthase 1 (platelet, cytochrome P450, subfamily V). This gene has LocusID: 6916, and is located on chromosome 7 with reported cytogenetic location 7q34-q35. The gene resides in genomic locus NT_{—}007914 (NCBI Genome Annotation). The gene product is a member of the cytochrome P450 superfamily of enzymes. The cytochrome P450 proteins are monooxygenases which catalyze many reactions involved in drug metabolism and synthesis of cholesterol, steroids and other lipids. TBXAS1 gene product is considered a member of the cytochrome P450 superfamily on the basis of sequence similarity rather than functional similarity. It is an endoplasmic reticulum membrane protein, and can catalyze the conversion of prostglandin H2 to thromboxane A2, a potent vasoconstrictor and inducer of platelet aggregation. TBXAS1 gene product may play a role in several pathophysiological processes including hemostasis, cardiovascular disease, and stroke. The gene expresses at least two transcript variants.

CPS 99 corresponds to DPYD which encodes dihydropyrimidine dehydrogenase. This gene has LocusID: 1806, and is located on chromosome 1 with reported cytogenetic location 1p22. The gene resides in genomic locus NT_{—}034389 (NCBI Genome Annotation). Dihydropyrimidine dehydrogenase is a pyrimidine catabolic enzyme which is involved in the initial and rate-limiting step in the pathway of uracil and thymidine catabolism and also in the pathway leading to the formation of beta-alanine. The DPYD gene is a large gene of approximately 150 kb consisting of at least 23 exons encoding a protein of approximately 111-kDa. Genetic deficiency of DPYD enzyme results in an error in pyrimidine metabolism associated with thymine-uraciluria and an increased risk of toxicity in cancer patients receiving 5-flourouracil chemotherapy.

Affymetrix annotation suggests that CPS 100 corresponds to ECGF1 which encodes endothelial cell growth factor 1 (platelet-derived). Nucleotides 4 to 120 of M63193 (SEQ ID NO: 94) align with ECGF1. This gene has LocusID: 1890, and is located on chromosome 22 with reported cytogenetic location 22q13.33. The gene resides in genomic locus NT_{—}011526 (NCBI Genome Annotation).

CPS 100 aligns with SCO2 with at least 98% sequence identity. SCO2 encodes SCO cytochrome oxidase deficient homolog 2 (yeast). It has LocusID: 9997, and is located on chromosome 22 with reported cytogenetic location 22q13.33. The gene resides in genomic locus NT_{—}011526. Mammalian cytochrome c oxidase (COX) catalyzes the transfer of reducing equivalents from cytochrome c to molecular oxygen and pumps protons across the inner mitochondrial membrane. In yeast, two related COX assembly genes, yeast SCO1 and SCO2 (synthesis of cytochrome c oxidase), enable subunits 1 and 2 to be incorporated into the holoprotein. SCO2 is the human homolog of the yeast SCO2 gene.

Affymetrix annotation suggests that CPS 101 corresponds to TIMP1 which encodes tissue inhibitor of metalloproteinase 1 (erythroid potentiating activity, collagenase inhibitor). The gene has LocusID: 7076, and is located at chromosome Xp11.3-p11.23.

Blast search of the Entrez human genome sequence database shows that CPS 101 aligns with an intron sequence of SYN1 with at least 97% sequence identity. SYN1 encodes synapsin I, and has LocusID: 6853 with reported cytogenetic location Xp11.23. The gene resides in genomic locus NT_{—}011568. CPS 101 matches with the non-protein-coding strand of SYN 1.

CPS 102 corresponds to GSN which encodes gelsolin (amyloidosis, Finnish type). This gene has LocusID: 2934, and is located on chromosome 9 with reported cytogenetic location 9q33. The gene resides in genomic locus NT_{—}017568 (NCBI Genome Annotation). Gelsolin is a calcium-dependent protein which may function to sever and cap actin filaments.

CPS 103 corresponds to SECTM1 which encodes secreted and transmembrane 1. This gene has LocusID: 6398, and is located on chromosome 17 with reported cytogenetic location 17q25. The gene resides in genomic locus NT_{—}025911 (NCBI Genome Annotation). The gene product is a transmembrane and secreted protein with characteristics of a type 1a transmembrane protein. It is found in a perinuclear Golgi-like pattern and thought to be involved in hematopoietic or immune system processes. The gene product may have a role in hematopoiesis or immune processes.

CPS 104 corresponds to OLR1 which encodes oxidised low density lipoprotein (lectin-like) receptor 1. This gene has LocusID: 4973, and is located on chromosome 12 with reported cytogenetic location 12 p13.2-p12.3. The gene resides in genomic locus NT_{—}035207 (NCBI Genome Annotation). Lectin-like oxidized low density lipoprotein receptor is a member of the C-type lectin receptor family, and may be involved in degradation of oxidized LDL by vascular endothelial cells.

CPS 105 corresponds to D6S49E (LST1) which encodes leukocyte specific transcript 1. This gene has LocusID: 7940, and is located on chromosome 6 with reported cytogenetic location 6p21.3. The gene resides in genomic locus NT_{—}007592 (NCBI Genome Annotation). The gene product is expressed in leukocytes and induced by IFN-gamma. It possibly functions in the immune response of monocytes and T cells.

CPS 106 corresponds to JUNB which encodes jun B proto-oncogene. This gene has LocusID: 3726, and is located on chromosome 19 with reported cytogenetic location 19p13.2. The gene resides in genomic locus NT_{—}011176 (NCBI Genome Annotation). The gene product may participate in AP-1 transcriptional activation.

CPS 107 corresponds to PFC which encodes properdin P factor, complement. This gene has LocusID: 5199, and is located on chromosome X with reported cytogenetic location Xp11.3-p11.23. The gene resides in genomic locus NT_{—}011568 (NCBI Genome Annotation). The gene product contains a related type-I repeat sequence, and may play a role in complement-mediated clearance.

CPS 108 corresponds to POLR2E which encodes polymerase (RNA) II (DNA directed) polypeptide E (25 kD). This gene has LocusID: 5434, and is located on chromosome 19 with reported cytogenetic location 19p13.3. The gene resides in genomic locus NT_{—}011277 (NCBI Genome Annotation). This gene encodes a subunit of RNA polymerase II, the polymerase responsible for synthesizing messenger RNA in eukaryotes. The encoded subunit is shared by the other two DNA-directed RNA polymerases and is present in two-fold molar excess over the other polymerase subunits. An interaction between this subunit and a hepatitis virus transactivating protein has been demonstrated, suggesting that interaction between transcriptional activators and the polymerase can occur through this subunit. A pseudogene is located on chromosome 11.

CPS 109 corresponds to THBS1 which encodes thrombospondin 1. This gene has LocusID: 7057, and is located on chromosome 15 with reported cytogenetic location 1Sq15. The gene resides in genomic locus NT_{—}030828 (NCBI Genome Annotation). Thrombospondin-1 is a member of a family of adhesive molecules. It has a role in blood clotting and in angiogenesis.

CPS 110 corresponds to HK2 which encodes hexokinase 2. This gene has LocusID: 3099, and is located on chromosome 2 with reported cytogenetic location 2p13. The gene resides in genomic locus NT_{—}022184 (NCBI Genome Annotation). Hexokinases phosphorylate glucose to produce glucose-6-phosphate, thus committing glucose to the glycolytic pathway. HK2 gene encodes hexokinase 2, the predominant form found in skeletal muscle. The gene product localizes to the outer membrane of mitochondria. Expression of this gene is insulin-responsive, and studies in rat suggest that it is involved in the increased rate of glycolysis seen in rapidly growing cancer cells.

CPS 110 also aligns with a chromosomal region near LOC139132 with about 95% sequence identity. Both the chromosomal region and LOC139132 reside in genomic locus NT_{—}011597. LOC139132 encodes a protein similar to WW domain binding protein 11 (SH3 domain-binding protein SNP70) (Npw38-binding protein NpwBP), and is located at Xq13.2.

Affymetrix annotation suggests that CPS 111 corresponds to INSIG1 which encodes insulin induced gene 1. The gene has LocusID: 3638 and is located at chromosome 7q36.

Blast search of the Entrez human genome sequence database shows that CPS 111 has about 80-85% sequence identity with a chromosomal region near LOC131742. This chromosomal region and LOC131742 reside in genomic locus NT_{—}022554. LOC131742 has reported cytogenetic location 3p12.1-p11.2.

CPS 112 corresponds to HCK which encodes hemopoietic cell kinase. This gene has LocusID: 3055, and is located on chromosome 20 with reported cytogenetic location 20q11-q12. The gene resides in genomic locus NT_{—}028392 (NCBI Genome Annotation). The gene product can function as a non-receptor protein tyrosine kinase.

Affymetrix annotation suggests that CPS 113 corresponds to HP10390, alos known as TMEM4 which encodes transmembrane protein 4. The gene has LocusID: 10330, and is located at chromosome 12q15.

Blast search of the Entrez human genome sequence database shows that CPS 113 aligns with two regions with 100% sequence identity. The first region is located in an intron of TIMELESS. The second region is located 5′ to the polypeptide-coding sequence of TIMELESS. TIMELESS encodes timeless homolog (Drosophila). It has LocusID: 8914, and is located on chromosome 12 with reported cytogenetic location 12q12-q13. The gene resides in genomic locus NT_{—}009458 (NCBI Genome Annotation). The gene product may be involved in circadian oscillation autoregulation.

CPS 114 corresponds to UNK_U51712 (LAGY) which encodes lung cancer-associated Y protein. This gene has LocusID: 84525, and is located on chromosome 4 with reported cytogenetic location 4q11-q12. The gene resides in genomic locus NT_{—}022853 (NCBI Genome Annotation). Multiple alternatively spliced transcript variants have been described for this gene.

CPS 115 corresponds to which encodes KIAA0217 which encodes KIAA0217 protein. This gene has LocusID: 23185, and is located on chromosome 10 with reported cytogenetic location 10p15.3. The gene resides in genomic locus NT_{—}024115 (NCBI Genome Annotation).

CPS 116 corresponds to NCF1 which encodes neutrophil cytosolic factor 1 (47 kD, chronic granulomatous disease, autosomal 1). This gene has LocusID: 4687, and is located on chromosome 7 with reported cytogenetic location 7q11.23. The gene resides in genomic locus NT_{—}034886 (NCBI Genome Annotation). NCF1 gene product can produce a burst of superoxide which is delivered to the lumen of the neutrophil phagosome. Mutations in NCF1, as well as in other NADPH oxidase subunits, can result in chronic granulomatous disease.

CPS 116 also aligns with LOC220830 and LOC256379 with 95-99% sequence identity. Both genes reside in genomic locus NT_{—}007758 on chromosome 7. Both genes encode proteins similar to neutrophil cytosolic factor 1 (47 kD, chronic granulomatous disease, autosomal 1).

CPS 117 corresponds to KIAA1113 (TRIM33) which encodes tripartite motif-containing 33. This gene has LocusID: 51592, and is located on chromosome 1 with reported cytogenetic location 1p13.1. The gene resides in genomic locus NT_{—}019273 (NCBI Genome Annotation). The protein encoded by this gene is thought to be a transcriptional corepressor. The protein is a member of the tripartite motif family. The tripartite motif includes three zinc-binding domains, a RING, a B-box type 1 and a B-box type 2, and a coiled-coil region. At least three alternatively spliced transcript variants for this gene have been described.

CPS 118 corresponds to DKFZP566H073 which encodes DKFZP566H073 protein. This gene has LocusID: 26001, and is located on chromosome 17 with reported cytogenetic location 17p13.3. The gene resides in genomic locus NT_{—}033299 (NCBI Genome Annotation). The gene product contains a C3HC4 type (RING) zinc finger, and can mediate protein-protein interactions.

CPS 119 corresponds to ATP2B1 which encodes ATPase, Ca++ transporting, plasma membrane 1. This gene has LocusID: 490, and is located on chromosome 12 with reported cytogenetic location 12q21-q23. The gene resides in genomic locus NT_{—}009729 (NCBI Genome Annotation).

Nucleotides 305 to 338 of CPS 119 align with an intron sequence of FLJ14075 with 100% sequence identity. FLJ14075 encodes hypothetical protein FLJ14075, and has LocusID: 79954 with reported cytogenetic location 2p25.1.

CPS 120 corresponds to IFITM2 which encodes interferon induced transmembrane protein 2 (1-8D). This gene has LocusID: 10581, and is located on chromosome 11 with reported cytogenetic location 11p15.5. The gene resides in genomic locus NT_{—}009407 (NCBI Genome Annotation). The expression of this gene can be induced by interferon.

CPS 121 corresponds to MNDA which encodes myeloid cell nuclear differentiation antigen. This gene has LocusID: 4332, and is located on chromosome 1 with reported cytogenetic location 1q22. The gene resides in genomic locus NT_{—}004982 (NCBI Genome Annotation). The myeloid cell nuclear differentiation antigen can be detected in nuclei of cells of the granulocyte-monocyte lineage. A 200-amino acid region of the protein is similar to a region in the proteins encoded by a family of interferon-inducible mouse genes, designated Ifi-201, Ifi-202, and Ifi-203. The MNDA mRNA, which contains an interferon-stimulated response element in the 5-prime untranslated region, can be upregulated in human monocytes exposed to interferon alpha. MNDA is located within 2,200 kb of FCER1A, APCS, CRP, and SPTA1. In its pattern of expression or regulation, MNDA resembles IFI16, suggesting that these genes participate in blood cell-specific responses to interferons.

CPS 122 corresponds to BTN3A2 which encodes butyrophilin, subfamily 3, member A2. This gene has LocusID: 11118, and is located on chromosome 6 with reported cytogenetic location 6p22.1. The gene resides in genomic locus NT_{—}007592 (NCBI Genome Annotation).

Nucleotides 213 to 250 of CPS 122 align with BTN3A3 and BTN3A1 with 94% and 97% sequence identity, respectively. BTN3A3 encodes butyrophilin, subfamily 3, member A3, and has LocusID: 10384. BTN3A1 encodes butyrophilin, subfamily 3, member A1, and has LocusID: 11119. Both genes are located at chromosome 6p22.1.

CPS 123 corresponds to KIAA0776 which encodes KIAA0776 protein. This gene has LocusID: 23376, and is located on chromosome 6 with reported cytogenetic location 6q16.3. The gene resides in genomic locus NT_{—}019424 (NCBI Genome Annotation).

CPS 124 corresponds to D6S2245E (HSD17B8) which encodes hydroxysteroid (17-beta) dehydrogenase 8. This gene has LocusID: 7923, and is located on chromosome 6 with reported cytogenetic location 6p21.3. The gene resides in genomic locus NT_{—}007592 (NCBI Genome Annotation). The protein encoded by this gene is similar to mouse Ke6 and is a member of the short-chain dehydrogenase superfamily. An alternatively spliced transcript of this gene has been detected.

CPS 125 corresponds to KIAA0628. This gene has LocusID: 9831, and is located on chromosome 8 with reported cytogenetic location 8q24.3. The gene resides in genomic locus NT_{—}023684 (NCBI Genome Annotation). CPS 125 is in the 3′ UTR of the gene.

CPS 126 corresponds to SELL which encodes selectin L (lymphocyte adhesion molecule 1). This gene has LocusID: 6402, and is located on chromosome 1 with reported cytogenetic location 1q23-q25. The gene resides in genomic locus NT_{—}034405 (NCBI Genome Annotation). Selectin L is a cell surface component that is a member of a family of adhesion/homing receptors which are involved in leukocyte-endothelial cell interactions. Selectin L is composed of multiple domains including one domain homologous to lectins, one to epidermal growth factor, and two to the consensus repeat units found in C3/C4 binding proteins. The protein can attach lymphocytes to lymph node high endothelial venules.

CPS 127 corresponds to MPV17 which encodes MpV17 transgene, murine homolog, glomerulosclerosis. This gene has LocusID: 4358, and is located on chromosome 2 with reported cytogenetic location 2p23-p21. The gene resides in genomic locus NT_{—}005204 (NCBI Genome Annotation). The gene product is similar to murine Mpv17. It is a predicted membrane protein and may be associated with nephrotic syndrome.

CPS 128 corresponds to FABP5 which encodes fatty acid binding protein 5 (psoriasis-associated). This gene has LocusID: 2171, and is located on chromosome 8 with reported cytogenetic location 8q21.13. The gene resides in genomic locus NT_{—}007972 (NCBI Genome Annotation). The gene product can be found in epidermal cells, and was identified as being upregulated in psoriasis tissue. Fatty acid binding proteins (FABPs) are a family of small, conserved, cytoplasmic proteins that bind long-chain fatty acids and other hydrophobic ligands. It is thought that FABPs roles include fatty acid uptake, transport, and metabolism.

Nucleotides 1 to 260 of CPS 128 have 100% sequence identity with an intron sequence of STX3A. STX3A encodes syntaxin 3A, and has LocusID: 6809 with reported cytogenetic location 11q12.3. STX3A resides in genomic locus NT_{—}033903. The alignment between CPS 128 and STX3A is in the non-protein-coding of the gene. Nucleotides 1 to 260 of CPS 128, or fragments thereof, also align to various regions on chromosomes 7, 13 and 15 with 95-97% sequence identity. In addition, nucleotides 1-260 of CPS 128, or fragments thereof, align to various region on chromosomes 2, 4, 13, 15 and 22 with sequence identity 88-93%.

CPS 129 corresponds to CASP1 which encodes caspase 1, apoptosis-related cysteine protease (interleukin 1, beta, convertase). This gene has LocusID: 834, and is located on chromosome 11 with reported cytogenetic location 11q23. The gene resides in genomic locus NT_{—}009151 (NCBI Genome Annotation). The gene product is a member of the cysteine-aspartic acid protease (caspase) family. Sequential activation of caspases plays a central role in the execution-phase of cell apoptosis. Caspases exist as inactive proenzymes which undergo proteolytic processing at conserved aspartic residues to produce 2 subunits, large and small, that dimerize to form the active enzyme. The CASP1 gene product can proteolytically cleave and activate the inactive precursor of interlukin-1, a cytokine involved in the processes such as inflammation, septic shock, and wound healing. CASP1 gene has been shown to induce cell apoptosis and may function in various developmental stages. It may have a role in the pathogenesis of Huntington disease. Alternative splicing of this gene results in at least five transcript variants encoding distinct isoforms.

Nucleotides 1 to 487 of CPS 129, or fragments thereof, aligns with various chromosome 11 regions with sequence identity 86-95%. These regions include LOC120332 and LOC160131, both of which have reported cytogenetic location 11q22.3 and encode proteins similar to caspase 1, isoform beta precursor (interleukin 1-beta convertase) (interleukin 1-B converting enzyme) (IL1B-convertase).

CPS 130 corresponds to PTPN6 which encodes protein tyrosine phosphatase, non-receptor type 6. This gene has LocusID: 5777, and is located on chromosome 12 with reported cytogenetic location 12 p13. The gene resides in genomic locus NT_{—}035206 (NCBI Genome Annotation). The protein encoded by this gene is a member of the protein tyrosine phosphatase (PTP) family. PTPs are known to be signaling molecules that regulate a variety of cellular processes including cell growth, differentiation, mitotic cycle, and oncogenic transformation. N-terminal part of PTPN6 gene product contains two tandem Src homolog (SH2) domains, which act as protein phospho-tyrosine binding domains and mediate the interaction of the gene product with its substrates. PTPN6 gene product can be expressed in hematopoietic cells, and may function as a regulator of multiple signaling pathways in hematopoietic cells. The gene product has been shown to interact with, and dephosphorylate a wide spectrum of phospho-proteins involved in hematopoietic cell signaling. At least three alternatively spliced variants of this gene, which encode distinct isoforms, have been reported.

CPS 130 also aligns with a chromosomal region near REA with at least 98% sequence identity. REA encodes repressor of estrogen receptor activity. It has LocusID: 11331 and reported cytogenetic location 12p13.

CPS 131 corresponds to SKAP-HOM (SCAP2) which encodes src family associated phosphoprotein 2. This gene has LocusID: 8935, and is located on chromosome 7 with reported cytogenetic location 7p21-p15. The gene resides in genomic locus NT_{—}007819 (NCBI Genome Annotation). The protein encoded by this gene belongs to the src family kinases. The encoded protein is similar to the src family associated phosphoprotein 1 and may function as an adaptor protein. The encoded protein has coiled-coil and SH3 domains, and is thought to play a role in the src signaling pathway in various cells. In a mouse study the SCAP2 gene is implicated in the processes of myeloid differentiation and growth arrest.

CPS 133 corresponds to GRO2 which encodes GRO2 oncogene. This gene has LocusID: 2920, and is located on chromosome 4 with reported cytogenetic location 4q21. The gene resides in genomic locus NT_{—}006216 (NCBI Genome Annotation). GRO2 is similar to GRO1. The gene product is a chemotactic agent for polymorphonuclear leukocytes.

CPS 133 also aligns with GRO1 with 85% sequence identity. GRO1 encodes GRO1 oncogene (melanoma growth stimulating activity, alpha), and has LocusID: 2919 with reported cytogenetic location 4q21. The gene resides in genomic locus NT_{—}006216.

CPS 134 corresponds to RRAS which encodes related RAS viral (r-ras) oncogene homolog. This gene has LocusID: 6237, and is located on chromosome 19 with reported cytogenetic location 19q13.3-qter. The gene resides in genomic locus NT_{—}011109 (NCBI Genome Annotation). The gene product is a member of the ras family of GTP binding proteins.

CPS 135 corresponds to KIAA0022 which encodes KIAA0022 gene product. This gene has LocusID: 9936, and is located on chromosome 2 with reported cytogenetic location 2q24.2. The gene resides in genomic locus NT_{—}005151 (NCBI Genome Annotation).

CPS 136 corresponds to FLII which encodes flightless I homolog (Drosophila). This gene has LocusID: 2314, and is located on chromosome 17 with reported cytogenetic location 17 p11.2. The gene resides in genomic locus NT_{—}030843 (NCBI Genome Annotation). The gene product is a homolog of Drosophila flightless I. It has a gelsolin-like actin-binding domain and a leucine-rich interaction domain.

CPS 137 corresponds to PRF1 which encodes perforin 1 (pore forming protein). This gene has LocusID: 5551, and is located on chromosome 10 with reported cytogenetic location 10q22. The gene resides in genomic locus NT_{—}024037 (NCBI Genome Annotation). Perforin is a cytolytic, channel-forming protein which plays a role in clearing virally infected host cells and tumor cells.

CPS 138 corresponds to KIAA0102 which encodes KIAAO102 gene product. This gene has LocusID: 9789, and is located on chromosome 11 with reported cytogenetic location 11q13.3. The gene resides in genomic locus NT_{—}033927 (NCBI Genome Annotation).

CPS 138 also aligns with a chromosomal region near PTAFR with 99% sequence identity. PTAFR encodes platelet-activating factor receptor, and has LocusID: 5724 and reported cytogenetic location 1p35-p34.3. It resides in genomic locus NT_{—}028053. Platelet-activating factor receptor is a G protein-coupled receptor.

In addition, CPS 138 has 86-94% sequence identity with various regions in chromosomes 5, 10, 11 and 15. For instance, CPS 138 aligns with a chromosome 15 region with 94% sequence identity. The chromosome 15 region includes LOC255320 which encodes a protein similar to microsomal signal peptidase 25 kDa subunit (SPase 25 kDa subunit) (SPC25).

CPS 139 corresponds to UNK_AA176780 (HSA249128) which encodes DIPB protein. This gene has LocusID: 54765, and is located on chromosome 11 with reported cytogenetic location 11p11.2. The gene resides in genomic locus NT_{—}009237 (NCBI Genome Annotation). The gene product is member of the B-box zinc finger family, and contains a region of low similarity to a region of murine Mid2. CPS 139 aligns with the region 3′ to the protein-coding strand of UNK_AA176780.

CPS 140 corresponds to PYGL which encodes phosphorylase, glycogen; liver (Hers disease, glycogen storage disease type VI). This gene has LocusID: 5836, and is located on chromosome 14 with reported cytogenetic location 14q21-q22. The gene resides in genomic locus NT_{—}025892 (NCBI Genome Annotation).

CPS 141 corresponds to DBP which encodes D site of albumin promoter (albumin D-box) binding protein. This gene has LocusID: 1628, and is located on chromosome 19 with reported cytogenetic location 19q13.3. The gene resides in genomic locus NT_{—}011109 (NCBI Genome Annotation). Albumin D-site-binding protein is a transcription factor which may play a role in the diurnal regulation of liver-specific genes. It is a member of the PAR (proline and acidic amino acid-rich) b/ZIP family.

CPS 143 corresponds to RANBP2L1 which encodes RAN binding protein 2-like 1. This gene has LocusID: 84220, and is located on chromosome 2 with reported cytogenetic location 2412.3. The gene resides in genomic locus NT_{—}022135 (NCBI Genome Annotation). RAN is a small GTP-binding protein of the RAS superfamily that is associated with the nuclear membrane and is thought to control a variety of cellular functions through its interactions with other proteins. RANBP2L1 gene shares sequence similarity with RANBP2, a large RAN-binding protein localized at the cytoplasmic side of the nuclear pore complex. It is believed that this RANBP2 gene family member arose from a duplication event 3 Mb distal to RANBP2. Alternative splicing has been observed for this locus and two variants are described. Additional splicing is suggested.

CPS 143 also aligns with LOC220692 with 100% sequence identity. LOC220692 encodes protein similar to RAN-binding protein 2-like 1, isoform 1 (sperm membrane protein BS-63). The gene is located at chromosome 2q12.3 and resides in genomic locus NT_{—}022135 (NCBI Genome Annotation).

In addition, CPS 143 has 94-96% sequence identity with KIAA0336, a chromosomal region near LOC256197, and an intron of LOC150821. KIAA0336 encodes KIAA0336 gene product, and has LocusID: 9648 and reported cytogenetic location 2q12.2. LOC256197 encodes a protein similar to ribosomal protein L22, and is located on chromosome 2. LOC150821 encodes a protein similar to KIAA0336 gene product, and is located at chromosome 2p 11.1.

CPS 144 corresponds to TNFSF10 which encodes tumor necrosis factor (ligand) superfamily, member 10. This gene has LocusID: 8743, and is located on chromosome 3 with reported cytogenetic location 3q26. The gene resides in genomic locus NT_{—}025667 (NCBI Genome Annotation). Tumor necrosis factor (TNF) family cytokines function as mediators of immune regulation and the inflammatory response. TNFSF10 gene product is a Type II glycoprotein of the tumor necrosis factor ligand superfamily, and can mediate cell death.

CPS 145 corresponds to KIAA0854 which encodes KIAA0854 protein. This gene has LocusID: 22882, and is located on chromosome 8 with reported cytogenetic location 8q24.13. The gene resides in genomic locus NT_{—}023663 (NCBI Genome Annotation).

CPS 146 corresponds to SDS which encodes serine dehydratase. This gene has LocusID: 10993, and is located on chromosome 12 with reported cytogenetic location 12q24.12. The gene resides in genomic locus NT_{—}009601 (NCBI Genome Annotation). Serine dehydratase catalyzes the PLP-dependent alpha,beta-elimination of L-serine to pyruvate and ammonia. It is one of three enzymes that are regarded as metabolic exits of the serine-glycine pool. Serine dehydratase can be found in the liver.

CPS 147 corresponds to KIAA0576 (COASTER) which encodes coactivator for steroid receptors. This gene has LocusID: 26036, and is located on chromosome 6 with reported cytogenetic location 6p1.1. The gene resides in genomic locus NT_{—}007592 (NCBI Genome Annotation).

Affymetrix annotation suggests that CPS 148 corresponds to FCGR3B which encodes Fc fragment of IgG, low affinity IIIb, receptor for (CD16). The gene has LocusID: 2215, and is located at chromosome 1q23.

Blast search of the Entrez human genome sequence database shows that CPS 148 aligns with FCGR3A with at least 97% sequence identity. FCGR3A encodes Fc fragment of IgG, low affinity IIIa, receptor for (CD16). The gene has LocusID: 2214, and is located on chromosome 1 with reported cytogenetic location 1q23. The gene resides in genomic locus NT_{—}004668 (NCBI Genome Annotation). The gene product is a Type III Fc gamma receptor and a member of the immunoglobulin superfamily. It can associate with zeta chain of the T-cell receptor complex (CD3Z).

CPS 149 corresponds to FCER1A which encodes Fc fragment of IgE, high affinity I, receptor for; alpha polypeptide. The gene product is the alpha subunit of the high affinity IgE receptor, and may be involved in triggering allergic responses. This gene has LocusID: 2205, and is located on chromosome 1 with reported cytogenetic location 1q23. The gene resides in genomic locus NT_{—}004982 (NCBI Genome Annotation). The IgE receptor contains 3 subunits: alpha, beta (MIM 147138), and gamma (MIM 147139). The alpha subunit can be glycosylated.

CPS 150 corresponds to CD44 which encodes CD44 antigen (homing function and Indian blood group system). This gene has LocusID: 960, and is located on chromosome 11 with reported cytogenetic location 11 p13. The gene resides in genomic locus NT_{—}009237 (NCBI Genome Annotation).

CPS 151 corresponds to ID1 which encodes inhibitor of DNA binding 1, dominant negative helix-loop-helix protein. This gene has LocusID: 3397, and is located on chromosome 20 with reported cytogenetic location 20q11. The gene resides in genomic locus NT_{—}028392 (NCBI Genome Annotation). The gene product is a member of the Id helix-loop-helix family of proteins, and may negatively regulate cell differentiation.

Blast search of the Entrez human genome sequence database shows that CPS 152 overlaps KIAA0963. CPS 152 aligns with the non-protein-coding strand of KIAA0963. KIAA0963 encodes KIAA0963 protein, and has LocusID: 22904 with reported cytogenetic location 19p13.3. KIAA0963 resides in genomic locus NT_{—}011277 (NCBI Genome Annotation).

CPS 153 corresponds to ADTB2 (AP2B1) which encodes adaptor-related protein complex 2, beta 1 subunit. This gene has LocusID: 163, and is located on chromosome 17 with reported cytogenetic location 17q11.2-q12. The gene resides in genomic locus NT_{—}010799 (NCBI Genome Annotation). The beta adaptin subunit is part of the clathrin coat assembly complex which links clathrin to receptors in coated pits and vesicles. These vesicles are involved in endocytosis and Golgi processing. The beta 1 subunit is one of the assembly proteins which binds to clathrin and initiates coat formation.

CPS 154 corresponds to MADH3 which encodes MAD, mothers against decapentaplegic homolog 3 (Drosophila). This gene has LocusID: 4088, and is located on chromosome 15 with reported cytogenetic location 15q21-q22. The gene resides in genomic locus NT_{—}010265 (NCBI Genome Annotation). The gene product is similar to murine Madh3. It is a member of the Smad family of proteins, and may affect transcription in response to TGF-beta signaling pathways.

CPS 155 corresponds to ARPCIB which encodes actin related protein 2/3 complex, subunit 1B (41 kD). This gene has LocusID: 10095, and is located on chromosome 7 with reported cytogenetic location 7q11.21. The gene resides in genomic locus NT_{—}007933 (NCBI Genome Annotation). The gene product is involved in assembly of the actin cytoskeleton, and may have a role in protrusion of lamellipodia.

CPS 155 also aligns with two chromosome 12 regions with 99-100% sequence identity. These two regions reside in genomic locus NT_{—}035283 (NCBI Genome Annotation). The first region is located near LOC196489 which encodes a protein similar to FLJ00209 protein. The second region is located near LOC144584 which encodes a protein similar to vacuolar protein sorting 35 (yeast); maternal-embryonic 3. Both LOC196489 and LOC144584 have reported cytogenetic location 12q13.12.

Affymetrix annotation suggests that CPS 156 corresponds to TALDO 1 which encodes transaldolase 1 and has LocusID: 6888. The gene is located at chromosome 11p15.5-p15.4.

CPS 157 corresponds to UNK_AL035079 (CAT) which encodes catalase. This gene has LocusID: 847, and is located on chromosome 11 with reported cytogenetic location 11p13. The gene resides in genomic locus NT_{—}009237 (NCBI Genome Annotation). Catalase is a tetrameric hemoprotein that can detoxify hydrogen peroxide.

CPS 158 corresponds to STXBP1 which encodes syntaxin binding protein 1. This gene has LocusID: 6812, and is located on chromosome 9 with reported cytogenetic location 9q34.1. The gene resides in genomic locus NT_{—}029366 (NCBI Genome Annotation).

CPS 159 corresponds to UNG2 which encodes uracil-DNA glycosylase 2. This gene has LocusID: 10309, and is located on chromosome 5 with reported cytogenetic location 5p15.2-p13.1. The gene resides in genomic locus NT_{—}006431 (NCBI Genome Annotation). Uracil-DNA glycosylase 2 possesses uracil-DNA glycosylase activity.

CPS 160 corresponds to DUSP5 which encodes dual specificity phosphatase 5. This gene has LocusID: 1847, and is located on chromosome 10 with reported cytogenetic location 10q25. The gene resides in genomic locus NT_{—}030059 (NCBI Genome Annotation). The protein encoded by DUSP5 gene is a member of the dual specificity protein phosphatase subfamily. These phosphatases can inactivate their target kinases by dephosphorylating both the phosphoserine/threonine and phosphotyrosine residues. They can negatively regulate members of the mitogen-activated protein (MAP) kinase superfamily (MAPK/ERK, SAPK/JNK, p38), which are associated with cellular proliferation and differentiation. Different members of the family of dual specificity phosphatases show distinct substrate specificities for various MAP kinases, different tissue distribution and subcellular localization, and different modes of inducibility of their expression by extracellular stimuli. DUSP5 gene product can inactivate ERK1, and is expressed in a variety of tissues including pancreas and brain.

CPS 161 corresponds to PTPRO which encodes protein tyrosine phosphatase, receptor type, O. This gene has LocusID: 5800, and is located on chromosome 12 with reported cytogenetic location 12p13.3-p13.2. The gene resides in genomic locus NT_{—}009714 (NCBI Genome Annotation). The gene product is an integral membrane protein containing a transmembrane domain and an intracellular catalytic domain with a characteristic signature motif. Several alternatively spliced transcript variants, some of which encode different isoforms of the protein, have been described. The gene product also contains fibronectin type III-like repeats and putative glycosylation sites in the extracellular domain.

Affymetrix annotation suggests that CPS 162 corresponds to SLA which encodes Src-like-adaptor and has LocusID: 6503. The gene has reported cytogenetic location at chromosome 8q24.

Blast search of the Entrez human genome sequence database shows that CPS 162 aligns with an intron sequence of TG with at least 99% sequence identity. TG encodes thyroglobulin, and has LocusID: 7038 with reported cytogenetic location 8q24.2-q24.3. The gene resides in genomic locus NT_{—}008150 (NCBI Genome Annotation). CPS 162 matches with the non-protein-coding strand of TG. Thyroglobulin is a precursor of thyroid hormones.

CPS 163 corresponds to RFP2 which encodes ret finger protein 2. This gene has LocusID: 10206, and is located on chromosome 13 with reported cytogenetic location 13q14. The gene resides in genomic locus NT_{—}033922 (NCBI Genome Annotation). The protein encoded by RFP2 gene is a member of the tripartite motif (TRIM) family. The TRIM motif includes three zinc-binding domains, a RING, a B-box type 1 and a B-box type 2, and a coiled-coil region. The RFP2 gene product localizes to cytoplasmic bodies near the nucleus. The gene is located on chromosome 13 within the minimal deletion region for B-cell chronic lymphocytic leukemia. Alternative splicing of this gene has been described.

Nucleotides 3 to 255 of CPS 163 have 83% sequence identity with a chromosomal region near LOC162162. LOC162162 encodes a protein similar to spinocerebellar ataxia type 1, and has reported cytogenetic location 16q23.1.

CPS 164 corresponds to ADFP which encodes adipose differentiation-related protein (adipophilin). This gene has LocusID: 123, and is located on chromosome 9 with reported cytogenetic location 9p21.2. The gene resides in genomic locus NT_{—}023974 (NCBI Genome Annotation). Adipocyte differentiation-related protein is associated with the globule surface membrane material. The protein is a major constituent of the globule surface. Increase in mRNA levels is one of the earliest indications of adipocyte differentiation. The product is also a component of milk lipid globules.

CPS 164 aligns with a chromosome 1 region near LOC254424 with 94% sequence identity. LOC254424 encodes a protein similar to adipophilin (adipose differentiation-related protein) (ADRP).

CPS 165 corresponds to ADTD (AP3D1) which encodes adaptor-related protein complex 3, delta 1 subunit. This gene has LocusID: 8943, and is located on chromosome 19 with reported cytogenetic location 19p13.3. The gene resides in genomic locus NT_{—}011268 (NCBI Genome Annotation). Delta-adaptin is a component of the AP-3 complex which is involved in intracellular transport.

Affymetrix annotation suggests that CPS 166 corresponds to ADTG which is also known as AP101. The gene encodes adaptor-related protein complex 1, gamma 1 subunit. The gene has LocusID: 164, and is located at chromosome 16q23.

Blast search of the Entrez human genome sequence database shows that CPS 166 aligns to LOC255980 with at least 99% sequence identity. LOC255980 encodes a protein similar to hypothetical protein FLJ20151, and is located on chromosome 15. LOC255980 resides in genomic locus NT_{—}010265. It overlaps FLJ20151 which has LocusID: 54837 and reported cytogenetic location 15q21.3.

CPS 168 corresponds to UNK_X87344 (PSMB8) Which encodes proteasome (prosome, macropain) subunit, beta type, 8 (large multifunctional protease 7). This gene has LocusID: 5696, and is located on chromosome 6 with reported cytogenetic location 6p21.3. The gene resides in genomic locus NT_{—}007592 (NCBI Genome Annotation). Beta subunit 8 of the proteasome (prosome macropain) can replace beta subunit PSMB5 when cells are stimulated by interferon gamma, thereby altering proteasome specificity.

CPS 170 corresponds to CAPG which encodes capping protein (actin filament), gelsolin-like. This gene has LocusID: 822, and is located on chromosome 2 with reported cytogenetic location 2cen-q24. The gene resides in genomic locus NT_{—}015805 (NCBI Genome Annotation). The gene product is a macrophage capping protein. It can reversibly block the barbed ends. The gene product is a member of the gelsolin/villin protein family.

CPS 171 corresponds to ARL7 which encodes ADP-ribosylation factor-like 7. This gene has LocusID: 10123, and is located on chromosome 2 with reported cytogenetic location 2q37.2. The gene resides in genomic locus NT_{—}005414 (NCBI Genome Annotation). ADP-ribosylation factor-like 7 (ARL7) is a member of the ADP-ribosylation factor family of GTP-binding proteins. ARL7 is similar to ARL4 and ARL6, and each has a nuclear localization signal and a high guanine nucleotide exchange rate.

CPS 172 corresponds to RCV1 which encodes recoverin. This gene has LocusID: 5957, and is located on chromosome 17 with reported cytogenetic location 17p13.1. The gene resides in genomic locus NT_{—}010718 (NCBI Genome Annotation). Recoverin is a calcium-binding protein which can activate guanylate cyclase activity.

CPS 173 corresponds to GRO3 which encodes GRO3 oncogene. This gene has LocusID: 2921, and is located on chromosome 4 with reported cytogenetic location 4q21. The gene resides in genomic locus NT_{—}006216 (NCBI Genome Annotation). GRO3 is similar to human GRO 1, and may encode a mitogenic factor.

CPS 175 corresponds to UNK_AF070606 (ATP2B 1) which encodes ATPase, Ca++ transporting, plasma membrane 1. This gene has LocusID: 490, and is located on chromosome 12 with reported cytogenetic location 12q21-q23. The gene resides in genomic locus NT_{—}009729 (NCBI Genome Annotation). CPS 175 aligns with the 3′ UTR of the protein-coding strand of the gene.

CPS 176 corresponds to AOAH which encodes acyloxyacyl hydrolase (neutrophil). This gene has LocusID: 313, and is located on chromosome 7 with reported cytogenetic location 7 p14-p12. The gene resides in genomic locus NT_{—}007819 (NCBI Genome Annotation). Acyloxyacyl hydrolase is a 2-subunit lipase which can hydrolyze the secondary (acyloxyacyl-linked) fatty acyl chains from the lipid A region of bacterial endotoxins. Acyloxyacyl hydrolase may modulate host inflammatory responses to gram-negative bacterial invasion. The 2 subunits are encoded by a single mRNA.

CPS 177 corresponds to UNK_AJ224442 (WBSCR22) which encodes Williams Beuren syndrome chromosome region 22. This gene has LocusID: 114049, and is located on chromosome 7. The gene resides in genomic locus NT_{—}007758 (NCBI Genome Annotation).

CPS 178 corresponds to MSR1 which encodes macrophage scavenger receptor 1. This gene has LocusID: 4481, and is located on chromosome 8 with reported cytogenetic location 8p22. The gene resides in genomic locus NT_{—}015280 (NCBI Genome Annotation). This gene encodes the class A macrophage scavenger receptors, which include at least three different types (1, 2, 3) generated by alternative splicing of this gene. These receptors or isoforms are macrophage-specific trimeric integral membrane glycoproteins and have been implicated in many macrophage-associated physiological and pathological processes including atherosclerosis, Alzheimer's disease, and host defense. The isoforms type 1 and type 2 are functional receptors and are able to mediate the endocytosis of modified low density lipoproteins (LDLs). The isoform type 3 may have an altered intracellular processing and can be trapped within the endoplasmic reticulum. The isoform type 3 can inhibit the function of isoforms type 1 and type 2 when co-expressed, indicating a dominant negative effect and suggesting a mechanism for regulation of scavenger receptor activity in macrophages.

CPS 179 corresponds to XAP4 (C20orf18) which encodes chromosome 20 open reading frame 18. This gene has LocusID: 10616, and is located on chromosome 20 with reported cytogenetic location 20p13. The gene resides in genomic locus NT_{—}011387 (NCBI Genome Annotation). The gene contains at least 12 exons. Several alternatively spliced transcript variants have been described. The gene product is similar to mouse UIP28/UbcM4 interacting protein at the amino acid level, and contains a C3HC4 type (RING) zinc finger. It may interact with PKC and mediate protein-protein interactions.

CPS 180 corresponds to C1QR1 which encodes complement component 1, q subcomponent, receptor 1. This gene has LocusID: 22918, and is located on chromosome 20 with reported cytogenetic location 20p11.21. The gene resides in genomic locus NT_{—}011387 (NCBI Genome Annotation). The gene product is a type I membrane protein and can act as a receptor for complement protein C1q, mannose-binding lectin, and pulmonary surfactant protein A. The gene product is a functional receptor involved in ligand-mediated enhancement of phagocytosis.

CPS 181 corresponds to STAT4 which encodes signal transducer and activator of transcription 4. This gene has LocusID: 6775, and is located on chromosome 2 with reported cytogenetic location 2q32.2-q32.3. The gene resides in genomic locus NT_{—}022197 (NCBI Genome Annotation). The gene product is a member of the STAT family of transcription factors. In response to cytokines and growth factors, STAT family members may be phosphorylated by the receptor associated kinases, and then form homo- or heterodimers that translocate to the cell nucleus where they act as transcription activators. STAT4 gene product may be involved in mediating responses to IL12 in lymphocytes and regulating the differentiation of T helper cells.

CPS 182 corresponds to UNK_AL049309 (SFRS12) which encodes splicing factor, arginine/serine-rich 12. This gene has LocusID: 140890, and is located on chromosome 5 with reported cytogenetic location 5q12.3. The gene resides in genomic locus NT_{—}006431 (NCBI Genome Annotation).

CPS 183 corresponds to RNASE2 which encodes ribonuclease, RNase A family, 2 (liver, eosinophil-derived neurotoxin). This gene has LocusID: 6036, and is located on chromosome 14 with reported cytogenetic location 14q24-q31. The gene resides in genomic locus NT_{—}025892 (NCBI Genome Annotation). The gene product is a member of ribonuclease superfamily, and has neurotoxic and ribonuclease activities.

CPS 183 also aligns with LOC122661 and RNASE3 with about 86-93% sequence identity. LOC122661 encodes a protein similar to nonsecretory ribonuclease precursor (Ribonuclease US) (Eosinophil-derived neurotoxin) (RNase UpI-2) (Ribonuclease 2) (RNase 2). It is located at chromosome 14q11.1, and resides in genomic locus NT_{—}025892. RNASE3 encodes ribonuclease, RNase A family, 3 (eosinophil cationic protein). RNASE3 has LocusID: 6037 and reported cytogenetic location 14q24-q31. It also resides in genomic locus NT_{—}025892. RNASE3 gene product is a member of the ribonuclease superfamily, and has both neurotoxic and ribonuclease activities.

CPS 184 corresponds to NCF4 which encodes neutrophil cytosolic factor 4 (40 kD). This gene has LocusID: 4689, and is located on chromosome 22 with reported cytogenetic location 22q13.1. The gene resides in genomic locus NT_{—}011520 (NCBI Genome Annotation). The protein encoded by this gene is a cytosolic element of nicotinamide adenine dinucleotide phosphate-oxidase. Upon neutrophil stimulation, the encoded protein and other cytosolic elements are sent to the cell membrane from the cytosol to form a complex which produces phagocytic oxygen radicals. Two motifs found in this protein, an SH3 domain and a PC motif, are significant to protein-protein interactions. Through interaction with the SH3 domain, NCF4 gene product is responsible for the downregulation of NADPH-oxidase. Alternative splicing has been observed in this gene. CPS 184 aligns to the 3′ UTR of the gene.

CPS 185 corresponds to ANXA4 which encodes annexin A4. This gene has LocusID: 307, and is located on chromosome 2 with reported cytogenetic location 2p13. The gene resides in genomic locus NT_{—}022184 (NCBI Genome Annotation). Annexin A4 belongs to the annexin family of calcium-dependent phospholipid binding proteins. Several members of the annexin family have been implicated in membrane-related events along exocytotic and endocytotic pathways. Annexin A4 may interact with ATP, have in vitro anticoagulant activity, and inhibit phospholipase A2 activity. Annexin A4 can be detected in epithelial cells.

CPS 186 corresponds to ME2 which encodes malic enzyme 2, NAD(+)-dependent, mitochondrial. This gene has LocusID: 4200, and is located on chromosome 18 with reported cytogenetic location 18q21. The gene resides in genomic locus NT_{—}033905 (NCBI Genome Annotation). The gene product is a homotetrameric protein which can catalyze the oxidative decarboxylation of malate to pyruvate.

CPS 183 also has 91% sequence identity with a chromosome 9 region near LOC169570. LOC169570 has reported cytogenetic location 9 p13.1.

CPS 187 corresponds to IF135 which encodes interferon-induced protein 35. This gene has LocusID: 3430, and is located on chromosome 17 with reported cytogenetic location 17q21. The gene resides in genomic locus NT_{—}035490 (NCBI Genome Annotation). Interferon-induced protein 35 associates with B-ATF transcription factor on interferon treatment. It contains a leucine-zipper motif.

CPS 188 corresponds to VAMP5 which encodes vesicle-associated membrane protein 5 (myobrevin). This gene has LocusID: 10791, and is located on chromosome 2 with reported cytogenetic location 2p11.2. The gene resides in genomic locus NT_{—}015805 (NCBI Genome Annotation). The gene product is a member of the synaptobrevin/VAMP family of proteins.

CPS 188 also has 87% sequence identity with an intron sequence of NSF. NSF encodes N-ethylmaleimide-sensitive factor. It has LocusID: 4905 and reported cytogenetic location 17q21.

CPS 189 corresponds to IMPA2 which encodes inositol(myo)-1(or 4)-monophosphatase 2. This gene has LocusID: 3613, and is located on chromosome 18 with reported cytogenetic location 18 p11.2. The gene resides in genomic locus NT_{—}010859 (NCBI Genome Annotation).

CPS 190 corresponds to GZMK which encodes granzyme K (serine protease, granzyme 3; tryptase II). This gene has LocusID: 3003, and is located on chromosome 5 with reported cytogenetic location 5q11-q12. The gene resides in genomic locus NT_{—}006431 (NCBI Genome Annotation). This gene product is a member of a group of related serine proteases from the cytoplasmic granules of cytotoxic lymphocytes. Cytolytic T lymphocytes (CTL) and natural killer (NK) cells can recognize, bind, and lyse specific target cells. They are thought to protect their host by lysing cells bearing on their surface “nonself” antigens, usually peptides or proteins resulting from infection by intracellular pathogens.

CPS 191 corresponds to BGN which encodes biglycan. This gene has LocusID: 633, and is located on chromosome X with reported cytogenetic location Xq28. The gene resides in genomic locus NT_{—}025965 (NCBI Genome Annotation). The protein encoded by this gene is a small cellular or pericellular matrix proteoglycan that is related in structure to two other small proteoglycans, decorin and fibromodulin. The encoded protein and decorin are thought to be the result of a gene duplication. Decorin contains one attached glycosaminoglycan chain, while biglycan probably contains two chains. Biglycan is thought to function in connective tissue metabolism by binding to collagen fibrils and transferring growth factor-beta. It may promote neuronal survival. This gene is a candidate gene for the Happle syndrome.

CPS 192 corresponds to AIF1 which encodes allograft inflammatory factor 1. This gene has LocusID: 199, and is located on chromosome 6 with reported cytogenetic location 6p21.3. The gene resides in genomic locus NT_{—}007592 (NCBI Genome Annotation). This gene is induced by cytokines and interferon. Its protein product is thought to be involved in negative regulation of growth of vascular smooth muscle cells, which contributes to the anti-inflammatory response to vessel wall trauma. The gene expresses at least three transcripts.

CPS 193 corresponds to CBP2 (SERPINH2) which encodes serine (or cysteine) proteinase inhibitor, clade H (heat shock protein 47), member 2. This gene has LocusID: 872, and is located on chromosome 11 with reported cytogenetic location 11q13.5. The gene resides in genomic locus NT_{—}033927 (NCBI Genome Annotation). The gene product is also known as Colligin-2, which is a collagen-binding protein that acts as a heat shock protein.

CPS 193 also has 92% sequence identity with a chromosome 9 region near pshsp47. Pshsp47 encodes serine (or cysteine) proteinase inhibitor, clade H (heat shock protein 47), member 2 pseudogene. It has LocusID: 158172 and reported cytogenetic location 9p 11.2.

Affymetrix annotation suggests that CPS 195 corresponds to IQGAP1 which encodes IQ motif containing GTPase activating protein 1. The gene has LocusID: 8826 with reported cytogenetic location 15q26.1.

Blast search of the Entrez human genome sequence database shows that nucleotides 395 to 5984 of L33075 align with IQGAP1 with 98% sequence identity. IQGAP1 encodes IQ motif containing GTPase activating protein 1. The gene has LocusID: 8826, and is located on chromosome 15 with reported cytogenetic location 15q26.1. The gene resides in genomic locus NT_{—}033276. The gene product contains a GTPase activating domain and multiple calmodulin binding domains, and can bind to actin cytoskeleton and inhibit GTPase activity of ras family of GTP binding proteins Cdc42Hs and rac.

CPS 196 corresponds to KIAA0823 (PPP1R16B) which encodes protein phosphatase 1, regulatory (inhibitor) subunit 16B. This gene has LocusID: 26051, and is located on chromosome 20 with reported cytogenetic location 20q11.23. The gene resides in genomic locus NT_{—}011362 (NCBI Genome Annotation).

CPS 197 corresponds to UNK_A1018098 (MGC15523) which encodes hypothetical protein MGC15523. This gene has LocusID: 124565, and is located on chromosome 17 with reported cytogenetic location 17q25.3. The gene resides in genomic locus NT_{—}010661 (NCBI Genome Annotation).

Affymetrix annotation suggests that CPS 198 corresponds to CD163. CD163 encodes CD163 antigen, and has LocusID: 9332. The gene is located at chromosome 12p13.3.

CPS 199 corresponds to HK3 which encodes hexokinase 3 (white cell). This gene has LocusID: 3101, and is located on chromosome 5 with reported cytogenetic location 5q35.2. The gene resides in genomic locus NT_{—}023132 (NCBI Genome Annotation). Hexokinases phosphorylate glucose to produce glucose-6-phosphate, thus committing glucose to the glycolytic pathway. HK3 gene encodes hexokinase 3 which is similar to hexokinases 1 and 2. Hexokinase 3 can be inhibited by its product glucose-6-phosphate.

CPS 200 corresponds to FOS which encodes v-fos FBJ murine osteosarcoma viral oncogene homolog. This gene has LocusID: 2353, and is located on chromosome 14 with reported cytogenetic location 14q24.3. The gene resides in genomic locus NT_{—}026437 (NCBI Genome Annotation). The Fos gene family has 4 members: FOS, FOSB, FOSL1, and FOSL2. These genes encode leucine zipper proteins that can dimerize with proteins of the JUN family, thereby forming the transcription factor complex AP-1. As such, the FOS proteins have been implicated as regulators of cell proliferation, differentiation, and transformation. In some cases, expression of the FOS gene has also been associated with apoptotic cell death. FOS gene product may also be involved in alteration of DNA methylation.

CPS 201 corresponds to DIFF48 (C6orf32) which encodes chromosome 6 open reading frame 32. This gene has LocusID: 9750, and is located on chromosome 6 with reported cytogenetic location 6p22.3-p21.32. The gene resides in genomic locus NT_{—}007592 (NCBI Genome Annotation). The protein encoded by DIFF48 can stimulate the formation of a non-mitotic multinucleate syncytium from proliferative cytotrophoblasts during trophoblast differentiation. An alternatively spliced transcript variant of this gene has been described.

CPS 202 corresponds to UNK_L23134 (GZMM) which encodes granzyme M (lymphocyte met-ase 1). This gene has LocusID: 3004, and is located on chromosome 19 with reported cytogenetic location 19p13.3. The gene resides in genomic locus NT_{—}011227 (NCBI Genome Annotation). Human natural killer (NK)-cells and activated lymphocytes express and store a distinct subset of neutral serine proteases together with proteoglycans and other immune effector molecules in large cytoplasmic granules. These serine proteases are collectively termed granzymes and include at least four distinct gene products: granzyme A, granzyme B, granzyme H, and Met-ase which is also known as granzyme M.

CPS 203 corresponds to LMO2 which encodes LIM domain only 2 (rhombotin-like 1). This gene has LocusID: 4005, and is located on chromosome 11 with reported cytogenetic location 11p13. The gene resides in genomic locus NT_{—}009237 (NCBI Genome Annotation). LMO2 encodes a cysteine-rich, two LIM-domain protein that is involved in yolk sac erythropoiesis. The LMO2 protein has a role in hematopoietic development. The LMO2 transcription start site is located approximately 25 kb downstream from the 11p13 T-cell translocation cluster (11p13 ttc), where a number T-cell acute lymphoblastic leukemia-specific translocations occur. The LMO2 protein is a member of the rhombotin family.

CPS 204 corresponds to PDI2 (PADI2) which encodes peptidyl arginine deiminase, type II. This gene has LocusID: 11240, and is located on chromosome 1 with reported cytogenetic location 1p35.2-p35.1. The gene resides in genomic locus NT_{—}034376 (NCBI Genome Annotation). The gene product is similar to rat skeletal muscle peptidyl arginine deiminase, type II. It may convert arginine residues within proteins to citrulline residues.

CPS 205 corresponds to UNK_AL031282 (FLJ13052) which encodes NAD kinase. This gene has LocusID: 65220, and is located on chromosome 1 with reported cytogenetic location 1p36.33-p36.21. The gene resides in genomic locus NT_{—}004350 (NCBI Genome Annotation).

CPS 205 also aligns with a chromosomal region near MMP23B with at least 98% sequence identity. MMP23B encodes matrix metalloproteinase 23B, and has LocusID: 8510 and reported cytogenetic location 1p36.3. The gene resides in genomic locus NT_{—}004350. CPS 205 aligns with the non-protein-coding strand of MMP23B.

CPS 206 corresponds to GABRG2 which encodes gamma-aminobutyric acid (GABA) A receptor, gamma 2. This gene has LocusID: 2566, and is located on chromosome 5 with reported cytogenetic location 5q31.1-q33.1. The gene resides in genomic locus NT_{—}030698 (NCBI Genome Annotation). The gamma-aminobutyric acid (GABA) A receptor, gamma 2 is found as an inhibitory neurotransmitter receptor in the brain.

CPS 207 corresponds to KIAA0404 which encodes KIAA0404 protein. This gene has LocusID: 23130, and is located on chromosome 11 with reported cytogenetic location 11q13.1. The gene resides in genomic locus NT_{—}033241 (NCBI Genome Annotation).

CPS 208 corresponds to UNK_U12471 (THBS1) which encodes thrombospondin 1. This gene has LocusID: 7057, and is located on chromosome 15 with reported cytogenetic location 15q15. The gene resides in genomic locus NT_{—}030828 (NCBI Genome Annotation). Thrombospondin-1 has a role in blood clotting and in angiogenesis. It is a member of a family of adhesive molecules.

Affymetrix annotation suggests that CPS 209 corresponds to IGHA1 which encodes immunoglobulin heavy constant alpha 1. The gene has LocusID: 3493 and reported cytogenetic location 14q32.33.

CPS 210 corresponds to ABCB1 which encodes ATP-binding cassette, sub-family B (MDR/TAP), member 1. This gene has LocusID: 5243, and is located on chromosome 7 with reported cytogenetic location 7q21.1. The gene resides in genomic locus NT_{—}007933 (NCBI Genome Annotation). The membrane-associated protein encoded by this gene is a member of the superfamily of ATP-binding cassette (ABC) transporters. ABC proteins transport various molecules across extra- and intra-cellular membranes. ABC genes are divided into seven distinct subfamilies (ABC 1, MDR/TAP, MRP, ALD, OABP, GCN20, White). This protein encoded by ABCB 1 gene is a member of the MDR/TAP subfamily. Members of the MDR/TAP subfamily are involved in multidrug resistance. The ABCB gene product is an ATP-dependent drug efflux pump for xenobiotic compounds with broad substrate specificity. It is responsible for decreased drug accumulation in multidrug-resistant cells and often mediates the development of resistance to anticancer drugs. It can also function as a transporter in the blood-brain barrier. The ABCB gene product is also known as P glycoprotein 1.

CPS 211 corresponds to NRIPI which encodes nuclear receptor interacting protein 1. This gene has LocusID: 8204, and is located on chromosome 21 with reported cytogenetic location 21q11.2. The gene resides in genomic locus NT_{—}011512 (NCBI Genome Annotation). Nuclear receptor interacting protein 1 is a nuclear protein that can interact with the hormone-dependent activation domain AF2 of nuclear receptors. Also known as RIP140, this protein modulates transcriptional activity of the estrogen receptor.

CPS 213 corresponds to GLB1 which encodes galactosidase, beta 1. This gene has LocusID: 2720, and is located on chromosome 3 with reported cytogenetic location 3p21.33. The gene resides in genomic locus NT_{—}005580 (NCBI Genome Annotation). The gene product can catalyze cleavage of the terminal galactose.

CPS 214 corresponds to MSC which encodes musculin (activated B-cell factor-1). This gene has LocusID: 9242, and is located on chromosome 8 with reported cytogenetic location 8q21. The gene resides in genomic locus NT_{—}034895 (NCBI Genome Annotation). The gene product contains a bHLH motif, a putative nuclear localization signal, a glycine-rich region, and a stretch of acidic residues. The gene product is capable of binding an E-box element either as a homodimer or as a heterodimer with E2A in vitro, and forms heterodimers with E2A proteins in vivo. It also contains a transcriptional repression domain and is capable of inhibiting the transactivation capability of E47, an E2A protein, in mammalian cells. MSC is thought to be a downstream target of the B-cell receptor signal transduction pathway.

CPS 216 corresponds to UNK_U95006 (MGC14480) which encodes hypothetical protein MGC14480. This gene has LocusID: 201254, and is located on chromosome 17 with reported cytogenetic location 17q25.3. The gene resides in genomic locus NT_{—}035479 (NCBI Genome Annotation).

Nucleotides 400 to 561 of CPS 216 also have 84% sequence identity with an intron sequence of EGI. EGI encodes endothelial-derived gene 1, and has LocusID: 80306 and reported cytogenetic location 4p 16.

CPS 217 corresponds to NK4 which encodes natural killer cell transcript 4. This gene has LocusID: 9235, and is located on chromosome 16 with reported cytogenetic location 16p13.3. The gene resides in genomic locus NT_{—}010552 (NCBI Genome Annotation). The gene product may play a role in cell adhesion. It contains an RGD motif.

CPS 217 also has 96% sequence identity with LOC124213 which encodes a protein similar to natural killer cell transcript 4. LOC124213 is located at chromosome 16p13.11, and also resides in genomic locus NT_{—}010552.

CPS 218 corresponds to UNK_Z24724 (FLJ20986) which encodes hypothetical protein FLJ20986. This gene has LocusID: 79572, and is located on chromosome 3 with reported cytogenetic location 3q29. The gene resides in genomic locus NT_{—}005535 (NCBI Genome Annotation). The alignment between CPS 218 and UNK_Z24724 is within an intron of GP5 which encodes glycoprotein V (platelet) and has LocusID: 2814.

CPS 219 corresponds to GRN which encodes granulin. This gene has LocusID: 2896, and is located on chromosome 17 with reported cytogenetic location 17q21.32. The gene resides in genomic locus NT_{—}010755 (NCBI Genome Annotation). Granulin is a putative growth factor. It is cysteine rich and contains multiple granulin repeats.

CPS 220 corresponds to EREG which encodes epiregulin. This gene has LocusID: 2069, and is located on chromosome 4 with reported cytogenetic location 4q13.3. The gene resides in genomic locus NT_{—}006216 (NCBI Genome Annotation). Epiregulin is a member of the epidermal growth factor family. Epiregulin can function as a ligand of EGFR (epidermal growth factor receptor), as well as a ligand of most members of the ERBB (v-erb-b2 oncogene homolog) family of tyrosine-kinase receptors. Epiregulin may promote cell proliferation.

CPS 221 corresponds to KIAA0382 (ARHGEF12) which encodes Rho guanine nucleotide exchange factor (GEF) 12. This gene has LocusID: 23365, and is located on chromosome 11 with reported cytogenetic location 11q23.3. The gene resides in genomic locus NT_{—}033899 (NCBI Genome Annotation). Rho GTPases play a role in numerous cellular processes that are initiated by extracellular stimuli that work through G protein coupled receptors. Rho guanine nucleotide exchange factor (GEF) 12 may form a complex with G proteins and stimulate Rho-dependent signals. This protein is observed to form myeloid/lymphoid fusion partner in acute myeloid leukemia.

CPS 222 corresponds to RNASE6 which encodes ribonuclease, RNase A family, k6. This gene has LocusID: 6039, and is located on chromosome 14 with reported cytogenetic location 14q11.1. The gene resides in genomic locus NT_{—}025892 (NCBI Genome Annotation). Ribonuclease k6 may function in host defense. It is a member of eosinophil-derived neurotoxin ribonuclease A superfamily.

CPS 223 corresponds to KIAA0442 which encodes autism-related protein 1. This gene has LocusID: 26053, and is located on chromosome 7 with reported cytogenetic location 7p13. The gene resides in genomic locus NT_{—}007758 (NCBI Genome Annotation).

Blast search of the Entrez human genome sequence database shows that nucleotides 35 to 1907 of CPS 225 align with various chromosomal regions with 89-90% sequence identity. These regions include LOC255718, LOC140070, and a chromosome 2 region between GPR39 and LOC255521. LOC255718 is located on chromosome 2 and has genomic locus NT_{—}034486 (NCBI Genome Annotation). LOC140070 encodes a protein similar to Protein CDC27Hs (Cell division cycle protein 27 homolog) (H-NUC). It is located on chromosome Yq11.1 and has genomic locus NT_{—}011878. GPR39 and LOC255521 reside in genomic locus NT_{—}034487. GPR39 encodes G protein-coupled receptor 39, and has LocusID: 2863 and reported cytogenetic location 2q21-q22.

Nucleotides 1317 to 1481 of CPS 225 have 98% sequence identity with a chromosome 12 region near LOC256746. LOC256746 encodes phosphodiesterase 3A, cGMP-inhibited. Nucleotides 1381 to 1418 and 1461 to 1502 have 100% sequence identity with an intron sequence of LOC 126500. LOC 126500 encodes a protein similar to hypothetical zinc finger protein KIAA 1473. It is located on chromosome 19p 13.11, and resides in genomic locus NT_{—}033317. Other fragments of CPS 225 have 84-93% sequence identity with regions on chromosomes 7, 11, 14, 18 and 21.

Blast search of the Entrez human genome sequence database shows that CPS 226 aligns to a region located in an intron of ATP8A. CPS 226 aligns with the non-protein-coding strand of ATP8A2. ATP8A2 encodes ATPase, aminophospholipid transporter-like, Class I, type 8A, member 2. ATP8A2 gene has LocusID: 51761, and is located on chromosome 13 with reported cytogenetic location 13q12-13. ATP8A2 resides in genomic locus NT_{—}009799 (NCBI Genome Annotation). ATP8A2 gene product may be a tumor suppressor. Loss of its function can convert cells to tumorigenic phenotype.

CPS 227 corresponds to ARHC which encodes ras homolog gene family, member C. This gene has LocusID: 389, and is located on chromosome 1 with reported cytogenetic location 1p21-p13. The gene resides in genomic locus NT_{—}019273 (NCBI Genome Annotation). The gene product can regulate reorganization of the actin cytoskeleton. CPS 227 aligns with the 3′ UTR region of ARHC. The alignment is also 3′ to MOV10 which encodes Mov10, Moloney leukemia virus 10, homolog (mouse). MOV10 has LocusID: 4343 and reported cytogenetic location 1p12. It resides in genomic locus NT_{—}019273.

CPS 228 corresponds to CREM which encodes cAMP responsive element modulator. This gene has LocusID: 1390, and is located on chromosome 10 with reported cytogenetic location 10p12.1-p11.1. The gene resides in genomic locus NT_{—}033896 (NCBI Genome Annotation). Cyclic AMP-responsive element modulator is a regulator of the transcription of cAMP-inducible genes.

CPS 229 corresponds to ISG15 which encodes interferon-stimulated protein, 15 kDa. This gene has LocusID: 9636, and is located on chromosome 1 with reported cytogenetic location 1p36.33. The gene resides in genomic locus NT_{—}004350 (NCBI Genome Annotation). The expression of ISG15 can be induced by interferon.

CPS 230 corresponds to CCR7 which encodes chemokine (C-C motif) receptor 7. This gene has LocusID: 1236, and is located on chromosome 17 with reported cytogenetic location 17q12-q21.2. The gene resides in genomic locus NT_{—}024901 (NCBI Genome Annotation). The gene product is a G protein-coupled receptor, and can bind to CC chemokine ELC and mediate intracellular calcium flux.

CPS 231 corresponds to F11 which encodes coagulation factor XI (plasma thromboplastin antecedent). This gene has LocusID: 2160, and is located on chromosome 4 with reported cytogenetic location 4q35. The gene resides in genomic locus NT_{—}022792 (NCBI Genome Annotation). Alternative splicing of the gene results in at least two transcripts. One transcript encodes the circulating plasma factor XI and an alternate transcript lacking exon 5 encodes the platelet factor XI. The plasma factor XI is present in plasma as a zymogen. The platelet factor XI is localized to platelets and megakaryocytes, and may play a role both in the maintenance of normal hemostasis and as a substitute for plasma factor XI.

Blast search of the Entrez human genome sequence database shows that CPS 232 aligns with a chromosome 4 region near LOC166760. This chromosome 14 region resides in genomic locus NT_{—}006344 (NCBI Genome Annotation). LOC166760 has reported cytogenetic location 4p 15.32.

CPS 233 corresponds to UNK_AC002073 (MGC17330) which encodes hypothetical protein MGC17330. This gene has LocusID: 113791, and is located on chromosome 22 with reported cytogenetic location 22q11.2-q22. The gene resides in genomic locus NT_{—}011520 (NCBI Genome Annotation).

CPS 234 corresponds to STK17A which encodes serine/threonine kinase 17a (apoptosis-inducing). This gene has LocusID: 9263, and is located on chromosome 7 with reported cytogenetic location 7p12-p14. The gene resides in genomic locus NT_{—}007819 (NCBI Genome Annotation). The alignment of CPS 234 and STK17A resides in an intron of FLJ10803. FLJ10803 encodes hypothetical protein FLJ10803, and has LocusID: 55744 and reported cytogenetic location 7p 15.1.

CPS 235 corresponds to BLVRA which encodes biliverdin reductase A. This gene has LocusID: 644, and is located on chromosome 7 with reported cytogenetic location 7p14-cen. The gene resides in genomic locus NT_{—}007819 (NCBI Genome Annotation).

CPS 236 corresponds to SPP1 which encodes secreted phosphoprotein 1 (osteopontin, bone sialoprotein I, early T-lymphocyte activation 1). This gene has LocusID: 6696, and is located on chromosome 4 with reported cytogenetic location 4q21-q25. The gene resides in genomic locus NT_{—}006204 (NCBI Genome Annotation). The gene product is a bone and blood vessel extracellular matrix protein involved in calcification and atherosclerosis.

CPS 237 corresponds to STX1A which encodes syntaxin 1A (brain). This gene has LocusID: 6804, and is located on chromosome 7 with reported cytogenetic location 7q11.23. The gene resides in genomic locus NT_{—}007758 (NCBI Genome Annotation). Syntaxin 1A (brain) may be involved in intracellular transport and neurotransmitter release.

CPS 238 corresponds to TKTL1 which encodes transketolase-like 1. This gene has LocusID: 8277, and is located on chromosome X with reported cytogenetic location Xq28. The gene resides in genomic locus NT_{—}025965 (NCBI Genome Annotation). Transketolase-like 1 is a thiamine pyrophosphate-dependent enzyme of pentose phosphate pathway.

CPS 239 corresponds to IER3 which encodes immediate early response 3. This gene has LocusID: 8870, and is located on chromosome 6 with reported cytogenetic location 6p21.3. The gene resides in genomic locus NT_{—}007592 (NCBI Genome Annotation). This gene functions in the protection of cells from Fas or tumor necrosis factor type alpha-induced apoptosis. Alternative splicing of this gene results in at least two transcript variants.

CPS 241 corresponds to PLAU which encodes plasminogen activator, urokinase. This gene has LocusID: 5328, and is located on chromosome 10 with reported cytogenetic location 10q24. The gene resides in genomic locus NT_{—}033890 (NCBI Genome Annotation). Urokinase plasminogen activator is a serine protease that cleaves plasminogen to form plasmin.

CPS 242 corresponds to ASS which encodes argininosuccinate synthetase. This gene has LocusID: 445, and is located on chromosome 9 with reported cytogenetic location 9q34.1. The gene resides in genomic locus NT_{—}008338 (NCBI Genome Annotation). The protein encoded by this gene catalyzes the penultimate step of the arginine biosynthetic pathway. There are approximately 10 to 14 copies of this gene including the pseudogenes scattered across the human genome, among which the one located on chromosome 9 appears to be the only functional gene for argininosuccinate synthetase. Mutations in the chromosome 9 copy of ASS cause citrullinemia. At least two alternatively spliced transcript variants of this gene have been reported.

CPS 242 also has 87-94% sequence identity with various regions on chromosome 2, 4, 5, 6, 7, 9, 11, 12 and X. These regions include LOC167449, LOC222906, ASSP2, ASSP3, ASSP4, ASSP5, LOC158452, LOC253843, LOC120341, LOC167519, and an intron of intron of LOC90957. LOC167449 encodes a protein similar to argininosuccinate synthetase and resides in genomic locus NT_{—}006431 with reported cytogenetic location 5q11.2. LOC222906 encodes a protein similar to argininosuccinate synthetase, and resides in genomic locus NT_{—}007819 with reported cytogenetic location 7p21.3. ASSP2 (LocusID: 447) encodes argininosuccinate synthetase pseudogene 2, and resides in genomic locus NT_{—}007592 with reported cytogenetic location 6p22.1. ASSP3 (LocusID: 448) encodes argininosuccinate synthetase pseudogene 3, and resides in genomic locus NT_{—}033256 with reported cytogenetic location 9q11-q22. ASSP4 (LocusID: 449) encodes argininosuccinate synthetase pseudogene 4, and resides in genomic locus NT_{—}025302 with reported cytogenetic location Xpter-p22. ASSP5 (LocusID: 450) encodes argininosuccinate synthetase pseudogene 5, and resides in genomic locus NT_{—}028405 with reported cytogenetic location Xq22-q26. LOC158452 encodes a protein similar to argininosuccinate synthetase, and resides in genomic locus NT_{—}023935 with reported cytogenetic location 9q21.31. LOC253843 encodes a protein similar to argininosuccinate synthetase, and resides in genomic locus NT_{—}006258 on chromosome 4. LOC120341 encodes a protein similar to argininosuccinate synthetase, and resides in genomic locus NT_{—}009151 with reported cytogenetic location 11q23.1. LOC167519 encodes a protein similar to argininosuccinate synthetase, and resides in genomic locus NT_{—}034778 with reported cytogenetic location 5q31.3. LOC90957 (LocusID: 90957) encodes DEAH-box RNA/DNA helicase AAM73547, and resides in genomic locus NT_{—}005367 with reported cytogenetic location 2p22.3.

Affymetrix annotation suggests that CPS 244 corresponds to DUXI which encodes double homeobox, 1. The gene has LocusID: 26584.

Blast search of the Entrez human genome sequence database shows that nucleotides 1 to 689 of AJ001481 (SEQ ID NO: 227) align with two chromosome 4 regions with 85-88% sequence identity. The first region is located 3′ to LOC131308. LOC131308 encodes a protein similar to FSHD Region Gene 2 protein. It has reported cytogenetic location 3p14.1 and resides in genomic locus NT_{—}022665. The second region is located near TRAP95. TRAP95 encodes thyroid hormone receptor-associated protein, 95-kD subunit. TRAP95 has LocusID: 10025 and reported cytogenetic location 19p13.3. It resides in genomic locus NT_{—}011277. TRAP95 gene product is a subunit of TRAP thyroid hormone receptor-associated protein complex, and functions as a coactivator for nuclear receptors.

CPS 245 corresponds to GPA33 which encodes glycoprotein A33 (transmembrane). This gene has LocusID: 10223, and is located on chromosome 1 with reported cytogenetic location 1q23.2. The gene resides in genomic locus NT_{—}004668 (NCBI Genome Annotation). The glycoprotein encoded by this gene is a cell surface antigen that is expressed in human colon cancers. The sequence of the extracellular region of the encoded protein contains 2 domains characteristic of the CD2 subgroup of the immunoglobulin (Ig) superfamily. The encoded protein may play a role in cell adhesion.

CPS 246 corresponds to PAI1 (SERPINE1) which encodes serine (or cysteine) proteinase inhibitor, clade E (nexin, plasminogen activator inhibitor type 1), member 1. This gene has LocusID: 5054, and is located on chromosome 7 with reported cytogenetic location 7q21.3-q22. The gene resides in genomic locus NT_{—}007933 (NCBI Genome Annotation). The gene product may regulate fibrinolysis. It is a member of the serpin family of serine protease inhibitors.

CPS 247 corresponds to CYP1B1 which encodes cytochrome P450, subfamily I (dioxin-inducible), polypeptide 1 (glaucoma 3, primary infantile). This gene has LocusID: 1545, and is located on chromosome 2 with reported cytogenetic location 2p21. The gene resides in genomic locus NT_{—}005367 (NCBI Genome Annotation). This gene product is a member of the cytochrome P450 superfamily of enzymes. The cytochrome P450 proteins are monooxygenases which catalyze many reactions involved in drug metabolism and synthesis of cholesterol, steroids and other lipids. The enzyme encoded by CYP1B1 gene localizes to the endoplasmic reticulum and metabolizes procarcinogens such as polycyclic aromatic hydrocarbons and 17beta-estradiol. Mutations in this gene have been associated with primary congenital glaucoma.

CPS 249 corresponds to HU-K5 (MGLL) which encodes monoglyceride lipase. This gene has LocusID: 11343, and is located on chromosome 3 with reported cytogenetic location 3q21.3. The gene resides in genomic locus NT_{—}005588 (NCBI Genome Annotation). The gene product may function in regulating levels of lysophospholipids.

Affymetrix annotation suggests that CPS 250 corresponds to DORA. DORA is also known as IGSF6 which encodes immunoglobulin superfamily, member 6. The gene has LocusID: 10261, and is located at chromosome 16p12-p13.

Blast search of the Entrez human genome sequence database shows that CPS 250 has at least 97% sequence identity with an intron sequence of DREVI. DREVI encodes CGI-81 protein. DREV1 has LocusID: 51108, and is located on chromosome 16 with reported cytogenetic location 16p 13-p 12. The gene resides in genomic locus NT_{—}010441 (NCBI Genome Annotation).

CPS 253 corresponds to MD-2 which encodes MD-2 protein. This gene has LocusID: 23643, and is located on chromosome 8 with reported cytogenetic location 8q13.2. The gene resides in genomic locus NT_{—}008209 (NCBI Genome Annotation). The MD-2 protein appears to associate with toll-like receptor 4 on the cell surface and confer responsiveness to lipopolysaccyaride (LPS), thus providing a link between the receptor and LPS signaling.

CPS 254 corresponds to UNK_U07563 (RRP4) which encodes homolog of yeast RRP4 (ribosomal RNA processing 4), 3′-5′-exoribonuclease. This gene has LocusID: 23404, and is located on chromosome 9 with reported cytogenetic location 9q34. The gene resides in genomic locus NT_{—}008338 (NCBI Genome Annotation). The gene product, also known as ribosomal RNA processing 4, is similar to S. cerevisiae RRP4 which is a component of both the nuclear and cytoplasmic forms of the ribosomal RNA processing 3′-5′ exosome complex. UNK_U07563 or RRP4 gene product may function in ribosomal RNA processing.

Affymetrix annotation suggests that CPS 255 corresponds to SPINK1 which encodes serine protease inhibitor, Kazal type 1. The gene has LocusID: 6690 and reported cytogenetic location 5q32.

Blast search of the Entrez human genome sequence database shows that CPS 255 has at least 97% sequence identity with a chromosome 5 region between LOC256135 and KIAA0555. Both LOC256135 and KIAA0555 reside in genomic locus NT_{—}006859 (NCBI Genome Annotation). LOC256135 encodes a protein similar to N-formyl peptide receptor. KIAA0555 encodes KIAA0555 gene product, and has LocusID: 9832 and reported cytogenetic location 5q32.

CPS 256 corresponds to UNK_S82470 (also known as BB1 or LENG4) which encodes leukocyte receptor cluster (LRC) member 4. This gene has LocusID: 79143, and is located on chromosome 19 with reported cytogenetic location 19q13.4. The gene resides in genomic locus NT_{—}011148 (NCBI Genome Annotation).

Affymetrix annotation suggests that CPS 257 corresponds to IGHG3. The gene encodes immunoglobulin heavy constant gamma 3 (G3m marker), and has LocusID: 3502. The gene is located at chromosome 14q32.33.

Blast search of the Entrez human genome sequence database shows that CPS 257 has at least 96% sequence identity with a chromosome 14 region near LOC122595. LOC122595 has reported cytogenetic location 14q32.33 and resides in genomic locus NT_{—}010168 (NCBI Genome Annotation).

CPS 260 corresponds to TNFRSF12 which encodes tumor necrosis factor receptor superfamily, member 12 (translocating chain-association membrane protein). This gene has LocusID: 8718, and is located on chromosome 1 with reported cytogenetic location 1p36.2. The gene resides in genomic locus NT_{—}028054 (NCBI Genome Annotation). The gene product contains a cytoplasmic death domain and transmembrane domains. It can induce apoptosis and activates NF-kappaB.

CPS 261 corresponds to UNK_AL031685 (KIAA0939) which encodes KIAA0939 protein. This gene has LocusID: 23315, and is located on chromosome 20 with reported cytogenetic location 20q13.13. The gene resides in genomic locus NT_{—}011362 (NCBI Genome Annotation).

CPS 262 corresponds to UNK_AL049963 (LOC64116) which encodes a protein up-regulated by BCG-CWS. This gene has LocusID: 64116, and is located on chromosome 4 with reported cytogenetic location 4q22-q24. The gene resides in genomic locus NT_{—}006383 (NCBI Genome Annotation).

Affymetrix annotation suggests that CPS 263 corresponds to APELIN which encodes apelin, a peptide ligand for APJ receptor. The gene has LocusID: 8862, and is located at chromosome Xq25-26.3.

Blast search of the Entrez human genome sequence database shows that CPS 263 aligns with OCRL with at least 97% sequence identity. OCRL refers to oculocerebrorenal syndrome of Lowe. This gene has LocusID: 4952, and is located on chromosome X with reported cytogenetic location Xq25-q26.1. The gene resides in genomic locus NT_{—}011786 (NCBI Genome Annotation). Mutations in this gene are linked to the disease oculocerebrorenal syndrome of Lowe. The encoded protein is a phospatidylinositol polyphosphate 5-phosphatase that can be found in golgi cistemae.

CPS 264 corresponds to VEGF which encodes vascular endothelial growth factor. This gene has LocusID: 7422, and is located on chromosome 6 with reported cytogenetic location 6p12. The gene resides in genomic locus NT_{—}007592 (NCBI Genome Annotation). Vascular endothelial growth factor can induce endothelial cell proliferation and vascular permeability.

CPS 265 corresponds to ELAVL2 which encodes ELAV (embryonic lethal, abnormal vision, Drosophila)-like 2 (Hu antigen B). This gene has LocusID: 1993, and is located on chromosome 9 with reported cytogenetic location 9p21. The gene resides in genomic locus NT_{—}023974 (NCBI Genome Annotation). The gene product can bind to 3′-untranslated regions of mRNA.

CPS 266 corresponds to PPP2R2A which encodes protein phosphatase 2 (formerly 2A), regulatory subunit B (PR 52), alpha isoform. This gene has LocusID: 5520, and is located on chromosome 8 with reported cytogenetic location 8p21.1. The gene resides in genomic locus NT_{—}023666 (NCBI Genome Annotation).

Nucleotides 163 to 505 of CPS 266 have 96% sequence identity with an intron sequence of TSC22. TSC22 encodes transforming growth factor beta-stimulated protein TSC-22. It has LocusID: 8848, and resides in genomic locus NT_{—}033922 with reported cytogenetic location 13q14. CPS 266 aligns with the non-protein-coding strand of the gene.

CPS 267 corresponds to UNK_AF001862 (FYB) which encodes FYN binding protein (FYB-120/130). This gene has LocusID: 2533, and is located on chromosome 5 with reported cytogenetic location 5 p13.1. The gene resides in genomic locus NT_{—}023195 (NCBI Genome Annotation). FYN-binding protein can modulate interleukin 2 production.

CPS 268 corresponds to OAS1 which encodes 2′,5′-oligoadenylate synthetase 1 (40-46 kD). This gene has LocusID: 4938, and is located on chromosome. 12 with reported cytogenetic location 12q24.1. The gene resides in genomic locus NT_{—}009770 (NCBI Genome Annotation). This gene product is a member of the 2′, 5′ oligoadenylate synthase family. It can be induced by interferons and catalyze the 2′, 5′ oligomers of adenosine in order to bind and activate RNase L. This gene family plays a significant role in the inhibition of cellular protein synthesis and viral infection resistance. Alternative splicing of OAS 1 gene produces at least two isoforms.

CPS 269 corresponds to CYBB which encodes cytochrome b-245, beta polypeptide (chronic granulomatous disease). This gene has LocusID: 1536, and is located on chromosome X with reported cytogenetic location Xp21.1. The gene resides in genomic locus NT_{—}011657 (NCBI Genome Annotation). Cytochrome b (−245) is composed of cytochrome b alpha (CYBA) and beta (CYBB) chain. It has been proposed as a primary component of the microbicidal oxidase system of phagocytes. CYBB deficiency is one of five described biochemical defects associated with chronic granulomatous disease (CGD). In this disorder, there is decreased activity of phagocyte NADPH oxidase. Neutrophils are able to phagocytize bacteria but cannot kill them in the phagocytic vacuoles. The cause of the killing defect may be an inability to increase the cell's respiration and consequent failure to deliver activated oxygen into the phagocytic vacuole.

CPS 270 corresponds to GFPT2 which encodes glutamine-fructose-6-phosphate transaminase 2. This gene has LocusID: 9945, and is located on chromosome 5 with reported cytogenetic location 5q34-q35. The gene resides in genomic locus NT_{—}006519 (NCBI Genome Annotation). The encoded protein is an enzyme of the hexosamine biosynthetic pathway.

Affymetrix annotation suggests that CPS 271 corresponds to BNIP3 which encodes BCL2/adenovirus E1B 19 kDa interacting protein 3. The gene has LocusID: 664, and is located at chromosome 14q11.2-q12.

Blast search of the Entrez human genome sequence database shows that CPS 271 has 100% sequence identity with an intron sequence of LOC159348. LOC159348 is located at chromosome 10q26.3 and has genomic locus NT_{—}024040 (NCBI Genome Annotation). Nucleotides 1 to 451 of CPS 270 have 97% sequence identity with a region 3′ to the protein-coding sequence of LOC254590. LOC254590 resides in genomic locus NT_{—}025892 on chromosome 14. In addition, nucleotides 4 to 450 align to a chromosome 15 region near CAPN3 with 81% sequence identity. CAPN3 encodes calpain 3, (p94), and has LocusID: 825 and reported cytogenetic location 15q15.1-q21.1. CAPN3 resides in genomic locus NT_{—}030828.

CPS 272 corresponds to BST1 which encodes bone marrow stromal cell antigen 1. This gene has LocusID: 683, and is located on chromosome 4 with reported cytogenetic location 4p 15. The gene resides in genomic locus NT_{—}006344 (NCBI Genome Annotation). Bone marrow stromal cell antigen 1 is a stromal cell line-derived glycosylphosphatidylinositol-anchored molecule that facilitates pre-B-cell growth. BST1 expression can be enhanced in bone marrow stromal cell lines derived from patients with rheumatoid arthritis. The polyclonal B-cell abnormalities in rheumatoid arthritis may be, at least in part, attributed to BST1 over-expression in the stromal cell population.

CPS 273 corresponds to UNK_U50535 (CG005) which encodes hypothetical protein from BCRA2 region. This gene has LocusID: 10443, and is located on chromosome 13 with reported cytogenetic location 13q12-q13. The gene resides in genomic locus NT_{—}009984 (NCBI Genome Annotation). The gene product has a region of low similarity to a region of rat 2′,3′-cyclic nucleotide 3′-phosphodiesterase

CPS 274 corresponds to CX3CR1 which encodes chemokine (CX3C) receptor 1. This gene has LocusID: 1524, and is located on chromosome 3 with reported cytogenetic location 3p21.3. The gene resides in genomic locus NT_{—}005498 (NCBI Genome Annotation). CX3C chemokine receptor is a G protein-coupled receptor. It can mediate leukocyte migration and adhesion, bind the CX3C chemokine fractalkine and signal through a pertussis toxin sensitive G-protein.

CPS 275 corresponds to CKTSF1B1 which encodes cysteine knot superfamily 1, BMP antagonist 1. This gene has LocusID: 26585, and is located on chromosome 15 with reported cytogenetic location 15q13-q15. The gene resides in genomic locus NT_{—}024680 (NCBI Genome Annotation). The gene product is a homolog of Xenopus laevis Gremlin which is a secreted protein that blocks signaling of bone morphogenetic protein (BMP) by preventing access to the BMP receptor.

CPS 276 corresponds to VNN2 which encodes vanin 2. This gene has LocusID: 8875, and is located on chromosome 6 with reported cytogenetic location 6q23-q24. The gene resides in genomic locus NT_{—}025741 (NCBI Genome Annotation). This gene product is a member of the Vanin family of proteins which share sequence similarity with each other, and also with biotimidase. The family includes secreted and membrane-associated proteins, a few of which have been reported to participate in hematopoietic cell trafficking. Members of this family possess pantetheinase activity, and may play a role in oxidative-stress response. Vanin 2 is a GPI-anchored cell surface molecule that plays a role in transendothelial migration of neutrophils. VNN2 gene lies in close proximity to, and in same transcriptional orientation as two other vanin genes on chromosome 6q23-q24. Two transcript variants encoding different isoforms have been described for this gene.

CPS 277 corresponds to KIAA0064 (SNX17) which encodes sorting nexin 17. This gene has LocusID: 9784, and is located on chromosome 2 with reported cytogenetic location 2p23-p22. The gene resides in genomic locus NT_{—}005204 (NCBI Genome Annotation).

CPS 278 corresponds to GRO1 which encodes GRO1 oncogene (melanoma growth stimulating activity, alpha). This gene has LocusID: 2919, and is located on chromosome 4 with reported cytogenetic location 4q21. The gene resides in genomic locus NT_{—}006216 (NCBI Genome Annotation). The gene product has melanoma growth stimulating activity. It may be a mitogenic factor involved in inflammatory processes.

CPS 278 has 88% sequence identity with GRO2 which encodes GRO2 oncogene. GRO2 has LocusID: 2920, and resides in genomic locus NT_{—}006216 with reported cytogenetic location 4q21.

CPS 279 corresponds to KIAA0655 (HIP12) which encodes huntingtin interacting protein 12. This gene has LocusID: 9026, and is located on chromosome 12 with reported cytogenetic location 12q24. The gene resides in genomic locus NT_{—}009464 (NCBI Genome Annotation).

Nucleotides 13 to 332 of CPS 279 aligns to an intron of DNAH 11 with 100% sequence identity. DNAH11 encodes dynein, axonemal, heavy polypeptide 11. It has LocusID: 8701, and
resides in genomic locus NT_{—}007819 with reported cytogenetic location 7p21. CPS 279 aligns with the non-protein-coding strand of DNAH11.

CPS 280 corresponds to RB1 which encodes retinoblastoma 1 (including osteosarcoma). This gene has LocusID: 5925, and is located on chromosome 13 with reported cytogenetic location 13q14.2. The gene resides in genomic locus NT_{—}033922 (NCBI Genome Annotation). Retinoblastoma protein 1 is a nuclear phosphoprotein with DNA binding activity. It can interact with histone deacetylase to repress transcription.

CPS 281 corresponds to KIAA0073 which encodes KIAA0073 protein. This gene has LocusID: 23398, and is located on chromosome 5 with reported cytogenetic location 5q12.3. The gene resides in genomic locus NT_{—}006431 (NCBI Genome Annotation).

CPS 282 corresponds to CAPN4 (CAPNS1) which encodes calpain, small subunit 1. This gene has LocusID: 826, and is located on chromosome 19 with reported cytogenetic location 19q13.13. The gene resides in genomic locus NT_{—}011296 (NCBI Genome Annotation). Calpains are a ubiquitous, well-conserved family of calcium-dependent, cysteine proteases. Calpain I and II are heterodimeric with distinct large subunits associated with common small subunits. CAPN4 (CAPNS I) gene encodes a small subunit common to both calpain I and II and is associated with myotonic dystrophy.

CPS 284 corresponds to MGSTIL1 (PTGES) which encodes prostaglandin E synthase. This gene has LocusID: 9536, and is located on chromosome 9 with reported cytogenetic location 9q34.3. The gene resides in genomic locus NT_{—}029366 (NCBI Genome Annotation). Prostaglandin (PG) E synthase is involved in eicosanoid and glutathione metabolism. It is a member of superfamily of membrane associated proteins.

CPS 285 corresponds to C1NH (SERPINGI) which encodes serine (or cysteine) proteinase inhibitor, clade G (C1 inhibitor), member 1, (angioedema, hereditary). This gene has LocusID: 710, and is located on chromosome 11 with reported cytogenetic location 11q12-q13.1. The gene resides in genomic locus NT_{—}033903 (NCBI Genome Annotation). The protein encoded by C1NH gene can inhibit activated C1r and C1s of the first complement component and thus regulate complement activation. Deficiency of the encoded protein may be associated with hereditary angioneurotic oedema (HANE). The encoded protein is a member of the serine protease inhibitor (serpin) superfamily.

Affymetrix annotation suggests that CPS 286 corresponds to H1FX. H1FX encodes H1 histone family, member X. It has LocusID: 8971.

CPS 287 corresponds to IL1B which encodes interleukin 1, beta. This gene has LocusID: 3553, and is located on chromosome 2 with reported cytogenetic location 2q14. The gene resides in genomic locus NT_{—}022135 (NCBI Genome Annotation). Interleukin 1 beta may initiate and amplify the immune and inflammatory responses.

CPS 288 corresponds to F3 which encodes coagulation factor III (thromboplastin, tissue factor). This gene has LocusID: 2152, and is located on chromosome 1 with reported cytogenetic location 1p22-p21. The gene resides in genomic locus NT_{—}021979 (NCBI Genome Annotation). Coagulation factor III which is a cell surface glycoprotein. This factor enables cells to initiate the blood coagulation cascades, and it functions as the high-affinity receptor for the coagulation factor VII. The resulting complex provides a catalytic event that is responsible for initiation of the coagulation protease cascades by specific limited proteolysis. There are at least three distinct domains of this factor: extracellular, transmembrane, and cytoplasmic. The factor functions in normal hemostasis, and is a component of the cellular immune response.

CPS 289 corresponds to DUSP4 which encodes dual specificity phosphatase 4. This gene has LocusID: 1846, and is located on chromosome 8 with reported cytogenetic location 8p12-p11. The gene resides in genomic locus NT_{—}030743 (NCBI Genome Annotation). The protein encoded by this gene is a member of the dual specificity protein phosphatase subfamily. These phosphatases inactivate their target kinases by dephosphorylating both the phosphoserine/threonine and phosphotyrosine residues. They negatively regulate members of the mitogen-activated protein (MAP) kinase superfamily (MAPK/ERK, SAPK/JNK, p38), which are associated with cellular proliferation and differentiation. DUSP4 gene product can inactivate ERK1, ERK2 and JNK, is expressed in a variety of tissues, and is localized in the nucleus. Two alternatively spliced transcript variants, encoding distinct isoforms, have been observed for this gene. In addition, multiple polyadenylation sites have been reported.

Affymetrix annotation suggests that CPS 290 corresponds to HUMRTVLH3. HUMRTVLH3 encodes endogenous retroviral protease. The gene has LocusID: 51354.

CPS 291 corresponds to UNK_AL049250 (BANP) which encodes BTG3 associated nuclear protein. This gene has LocusID: 54971, and resides in genomic locus NT_{—}010859 on chromosome 18 (NCBI Genome Annotation). The gene product can interact with CAF1, a component of the general transcription multisubunit complex. It is thought that BTG3 is involved in negative control of the cell cycle. The protein encoded by BTG3 gene binds to BTG3. Studies with mouse homolog suggest that this protein may also interact with a specific nuclear matrix/scaffold-associated region (MAR). Transcript variants encoding different isoforms have been described for this gene. The alignment between CPS 291 and BANP overlaps LOC124302 which encodes a protein similar to nuclear pore complex interacting protein. LOC124302 resides in genomic locus NT_{—}010859 with reported cytogenetic location 18p 11.1.

CPS 291 also aligns with other genes and chromosomal regions with at least 97% sequence identity. These genes and chromosomal regions include LOC92267, LOC146181, LOC146136, KIAA0220, LOC253666, and an intron of LOC220565, a region near LOC255019, a region near LOC83985, a region 5′ to the protein-coding sequence of NPIP, and a region 3′ to the protein-coding sequence of EIF3S8. LOC92267 encodes a protein similar to hypothetical protein FLJ12363. LOC92267 resides in NT_{—}010441 with reported cytogenetic location 16p12.1. CPS 291 aligns with the non-protein-coding strand of LOC92267. LOC146181 encodes a protein similar to Hypothetical protein KIAA0220, and resides in genomic locus NT_{—}010441 with reported cytogenetic location 16p12.2. LOC146136 encodes a protein similar to nuclear pore complex interacting protein, and resides in genomic locus NT_{—}010441 with reported cytogenetic location 16p12.1. KIAA0220 has LocusID: 23117, and resides in genomic locus NT_{—}035362 with reported cytogenetic location 16p12.1. LOC253666 has similarity to rat kidney-specific (KS) gene. LOC253666 resides in genomic locus NT_{—}010604 on chromosome 16. LOC220565 encodes a protein similar to PI-3-kinase-related kinase SMG-1, isoform 1; lambda/iota protein kinase C-interacting protein; phosphatidylinositol 3-kinase-related protein kinase. LOC220565 is located at chromosome 16q13, and resides in genomic locus NT_{—}033291. LOC255019 encodes a protein similar to nuclear pore complex interacting protein, and resides in genomic locus NT_{—}035360 on chromosome 16. LOC83985 encodes spinster-like protein, and has LocusID: 83985. LOC83985 resides in genomic locus NT_{—}035372 with reported cytogenetic location 16q13. NPIP (LocusID: 9284) encodes a nuclear pore complex interacting protein, and resides in genomic locus NT_{—}035359 with reported cytogenetic location 16p 13-p 11. EIF3 S8 encodes eukaryotic translation initiation factor 3, subunit 8 (110 kD). EIF3S8 has LocusID: 8663, and resides in genomic locus NT_{—}010589 with reported cytogenetic location 16p11.2. LOC146452 encodes a protein similar to KIAA0251 hypothetical protein. It resides in genomic locus NT_{—}010478 with reported cytogenetic location 16q22.3.

Nucleotides 1 to 196 of CPS 291 have about 86% sequence identity with LOC118735. LOC118735 encodes a protein similar to apoptosis response protein (prostate apoptosis response protein 4). LOC 118735 resides in NT_{—}030059 with reported cytogenetic location 10q24.2.

CPS 292 corresponds to UNK_AB002308 (KIAA0310) which encodes KIAA0310 gene product. This gene has LocusID: 9919, and is located on chromosome 9 with reported cytogenetic location 9q34.3. The gene resides in genomic locus NT_{—}033215 (NCBI Genome Annotation).

Affymetrix annotation suggests that CPS 293 corresponds to EDNI which encodes endothelin 1. The gene has LocusID: 1906, and is located at chromosome 6p24.1.

CPS 293 aligns with IL1B with at least 99% sequence identity. IL1B encodes interleukin 1, beta.

CPS 295 corresponds to FRAT2 which encodes frequently rearranged in advanced T-cell lymphomas 2. This gene has LocusID: 23401, and is located on chromosome 10 with reported cytogenetic location 10q23-q24.1. The gene resides in genomic locus NT_{—}03005 (NCBI Genome Annotation).

CPS 297 corresponds to IL8 which encodes interleukin 8. This gene has LocusID: 3576, and is located on chromosome 4 with reported cytogenetic location 4q13-q21. The gene resides in genomic locus NT_{—}006216 (NCBI Genome Annotation). Interleukin 8 is a cytokine that plays a role in chemoattraction and activation of neutrophils. It has similarity to several platelet-derived factors.

Affymetrix annotation suggests that CPS 298 corresponds to FSCN2. FSCN2 encodes fascin homolog 2, actin-bundling protein, retinal (Strongylocentrotus purpuratus). The gene has LocusID: 25794, and is located at chromosome 17q25.

CPS 300 corresponds to CAMP which encodes cathelicidin antimicrobial peptide. This gene has LocusID: 820, and is located on chromosome 3 with reported cytogenetic location 3p21.3. The gene resides in genomic locus NT_{—}022567 (NCBI Genome Annotation). Cathelicidin antimicrobial peptide is a precursor of a peptide with antibacterial activity.

CPS 301 corresponds to FCGR1A which encodes Fc fragment of IgG, high affinity Ia, receptor for (CD64). This gene has LocusID: 2209, and is located on chromosome 1 with reported cytogenetic location 1q21.2-q21.3. The gene resides in genomic locus NT_{—}032962 (NCBI Genome Annotation). The gene product, also known as Fc gamma R1, is a receptor for the Fc domain of IgG. It is a member of the immunoglobulin superfamily, and may have a role in immune response.

CPS 302 corresponds to MAT1A which encodes methionine adenosyltransferase I, alpha. This gene has LocusID: 0.4143, and is located on chromosome 10 with reported cytogenetic location 10q22. The gene resides in genomic locus NT_{—}033890 (NCBI Genome Annotation). Methionine adenosyltransferase I (alpha isoform) catalyzes the formation of S-adenosylmethionine from methionine and ATP. Both the beta and alpha isoforms may be encoded by MATIA. Methionine adenosyltransferase deficiency is known to be caused by recessive as well as dominant mutations, the latter identified in autosomal dominant persistant hypermethioninemia.

CPS 303 corresponds to CYP4B1 which encodes cytochrome P450, subfamily IVB, polypeptide 1. This gene has LocusID: 1580, and is located on chromosome 1 with reported cytogenetic location 1p34-p12. The gene resides in genomic locus NT_{—}004386 (NCBI Genome Annotation). This gene product is a member of the cytochrome P450 heme-binding monooxygenase superfamily, and can metabolize steroids, fatty acids and xenobiotics.

Affymetrix annotation suggests that CPS 304 corresponds to MUC3. MUC3 is also known as MUC3A, and encodes mucin 3A, intestinal. The gene has LocusID: 4584 and reported cytogenetic location 7q22.

Blast search of the Entrez human genome sequence database shows that CPS 304 aligns to regions 5′ to the protein-coding strand of MUC3B with 80-81% sequence identity. MUC3B encodes mucin 3B. It has LocusID: 57876 and reported cytogenetic location 7q22. The gene resides in genomic locus NT_{—}007933.

CPS 305 corresponds to B7 which encodes B7 protein. This gene has LocusID: 10233, and is located on chromosome 12 with reported cytogenetic location 12p13. The gene resides in genomic locus NT_{—}035206 (NCBI Genome Annotation). The gene product has similarity to the regulatory subunit of protein phosphatases. It contains leucine rich repeats, and may mediate protein-protein interactions.

CPS 306 corresponds to SCYA20 which encodes small inducible cytokine subfamily A (Cys-Cys), member 20. This gene has LocusID: 6364, and is located on chromosome 2 with reported cytogenetic location 2q33-q37. The gene resides in genomic locus NT_{—}005403 (NCBI Genome Annotation). The protein encoded by this gene is a chemotactic factor for lymphocytes.

CPS 307 corresponds to ILIRN which encodes interleukin 1 receptor antagonist. This gene has LocusID: 3557, and is located on chromosome 2 with reported cytogenetic location 2q14.2. The gene resides in genomic locus NT_{—}02213 (NCBI Genome Annotation). Interleukin 1 receptor antagonist binds to and inhibits the IL-1 receptor. It is a member of the interleukin-1 (IL-1) family.

CPS 308 corresponds to ZNF261 which encodes zinc finger protein 261. This gene has LocusID: 9203, and is located on chromosome X with reported cytogenetic location Xq13.1. The gene resides in genomic locus NT_{—}019696 (NCBI Genome Annotation). The gene product contains a putative zinc-binding motif (MYM).

CPS 309 corresponds to LGALS2 which encodes lectin, galactoside-binding, soluble, 2 (galectin 2). This gene has LocusID: 3957, and is located on chromosome 22 with reported cytogenetic location 22q13.1. The gene resides in genomic locus NT_{—}011520 (NCBI Genome Annotation). Galectin 2 is an actose-binding lectin involved in cell growth regulation.

CPS 310 corresponds to TGN51 (TGOLN2) which encodes trans-golgi network protein 2. This gene has LocusID: 10618, and is located on chromosome 2 with reported cytogenetic location 2p11.2. The gene resides in genomic locus NT_{—}015805 (NCBI Genome Annotation).

The biological mechanisms underlying the CCI-779 modulation of the expression levels of the CCI-779 activity genes have yet to be elucidated. Without being limited to any specific theory, the modulation may be attributed to the direct effect of CCI-779 on PBMCs or other blood cells. It may also be caused by the effect of CCI-779 on renal cell carcinoma tumors, which in turn induce the change of the gene expression profile in the peripheral blood cells.

Some of the CCI-779 activity genes are RCC disease genes that are differentially expressed in PBMCs of RCC patients relative to tumor-free humans. These genes include, for example, TUBB, CTSL, SCYA2, PAI2, UNK_AI679353, SCYA7, ILIR1, THBS1, NCF1, ATP2B1, GRO2, PRF1, DBP, FCGR3B, ADFP, GRO3, C1QR, RNASE2, CBP2, HK3, FOS, PDI2, UNK_U12471 (THBS1), EREG, SPP1, STX1A, TKTL1, DUX1, SPINK1, UNK_AL049963 (LOC64116), BNIP3, GRO1, IL1B, F3, UNK_AL049250, EDN1, FCGR1A, MUC3, B7, SCYA20, IL1RN, and ZNF261. Over the course of CCI-779 therapy, at least one subset of these genes, which are elevated or suppressed in RCC patients, return to the normal baseline levels.

Table 6 shows examples of RCC disease genes whose expression profiles in PBMCs can be modulated by CCI-779. Modulation of the expression profiles of these genes in ex vivo conditions is also indicated.

In one experiment, peripheral blood mononuclear cells isolated from tumor-free humans were treated with CCI-779 ex vivo. Comparison of the genes sensitive to CCI-779 treatment ex vivo with the CCI-779 activity genes of the present invention revealed a common set of genes. These genes represent surrogate markers of CCI-779 drug activity in vivo as well as ex vivo.

As appreciated by those skilled in the art, the above-described methodology can be employed to identify genes whose expression profiles in PBMCs can be modulated by other drugs.

TABLE 6
Modulation of RCC Disease Genes by CCI-779

| | | | RCC- | RCC | RCC 8 wks | RCC 16 wks | |
|---|---|---|---|---|---|---|---|
| | | | Free | Baseline | with CCI-779 | with CCI- | |
| | Entrez | | Average | Average | Average | 779 Average | |
| Gene Name | Accession | Gene Description | (n = 20) | (n = 21) | (n = 21) | (n = 21) | Ex Vivo Description |
| IL1R1 | M27492 | interleukin 1 receptor, type I | 6.20 | 17.86 | 11.29 | 4.00 | 2 fold induced by PHA, |
| | | | | | | | 2-fold downregulated |
| | | | | | | | by 300 nM CCI |
| EDN1 | J05008 | endothelin 1 | 14.55 | 104.62 | 111.95 | 16.00 | 2-fold downregulated |
| | | | | | | | by 300 nM CCI alone |
| | | | | | | | @ 24 h |
| ABL1 | M14752 | v-abl Abelson murine leukemia | 2.35 | 101.76 | 108.33 | 75.90 | 2-fold downregulated |
| | | viral oncogene homolog 1 | | | | | by 300 nM CCI alone |
| | | | | | | | @ 24 h |
| UNK_AF141349 | AF141349 | Tubulin, Beta | 6.55 | 14.00 | 17.33 | 22.48 | 2 fold induced by PHA, |
| | | | | | | | 2-fold downregulated |
| | | | | | | | by 300 nM CCI |
| UNK_AF141349 | AF141349 | Tubulin, Beta | 7.70 | 18.57 | 18.57 | 25.48 | 2 fold induced by PHA, |
| | | | | | | | 2-fold downregulated |
| | | | | | | | by 300 nM CCI |
| FCGR3B | X16863 | Fc fragment of IgG, low | 6.60 | 11.67 | 24.57 | 41.29 | 2-fold downregulated |
| | | affinity IIIb, receptor for | | | | | by 300 nM CCI alone |
| | | (CD16) | | | | | @ 24 h |
| UNK_M14087 | M14087 | Human HL14 gene encoding | 1.80 | 24.14 | 21.29 | 15.00 | 2-fold downregulated |
| | | beta-galactoside-binding lectin, | | | | | by 300 nM CCI alone |
| | | 3′ end, clone2 | | | | | @ 24 h |
| KIAA0168 | W28731 | KIAA0168 gene product | 1.80 | 18.90 | 17.71 | 14.05 | 2-fold downregulated |
| | | | | | | | by 300 nM CCI alone |
| | | | | | | | @ 24 h |
| UNK_M62896 | M62896 | Human lipocortin (LIP) 2 | 2.85 | 5.33 | 5.95 | 8.86 | 2-fold downregulated |
| | | pseudogene mRNA, complete | | | | | by 300 nM CCI alone |
| | | cds-like region | | | | | @ 24 h |
| MS4A3 | L35848 | membrane-spanning 4- | 1.95 | 7.05 | 6.90 | 8.52 | 2-fold downregulated |
| | | domains, subfamily A, member | | | | | by 300 nM CCI alone |
| | | 3 (hematopoietic cell-specific) | | | | | @ 24 h |
| SMARCA4 | U29175 | SWI/SNF related, matrix | 7.75 | 56.14 | 80.19 | 62.86 | 2-fold downregulated |
| | | associated, actin dependent | | | | | by 300 nM CCI alone |
| | | regulator of chromatin, | | | | | @ 24 h |
| | | subfamily a, member 4 | | | | | |
| BASP1 | AA135683 | brain acid-soluble protein 1 | 7.70 | 16.24 | 10.76 | 10.19 | 2 fold induced by PHA, |
| | | | | | | | 2-fold downregulated |
| | | | | | | | by 300 nM CCI |
| CHN2 | U07223 | chimerin (chimaerin) 2 | 3.75 | 11.29 | 11.86 | 10.86 | 2-fold downregulated |
| | | | | | | | by 300 nM CCI alone |
| | | | | | | | @ 24 h |
| FECH | D00726 | ferrochelatase (protoporphyria) | 2.15 | 26.71 | 28.10 | 24.24 | 2-fold downregulated |
| | | | | | | | by 300 nM CCI alone |
| | | | | | | | @ 24 h |
| SCYA2 | M28225 | small inducible cytokine A2 | 9.10 | 82.29 | 43.43 | 6.71 | 2-fold downregulated |
| | | (monocyte chemotactic protein | | | | | by 300 nM CCI alone |
| | | 1, homologous to mouse Sigje) | | | | | @ 24 h |
| AQP9 | AB008775 | aquaporin 9 | 9.40 | 24.57 | 19.76 | 15.76 | 2-fold downregulated |
| | | | | | | | by 300 nM CCI alone |
| | | | | | | | @ 24 h AND 2-fold |
| | | | | | | | induced by PHA, 2-fold |
| | | | | | | | downregulated by 300 nM |
| | | | | | | | CCI |
| TREX1 | AJ243797 | three prime repair exonuclease 1 | 2.90 | 9.29 | 12.90 | 16.19 | 2-fold downregulated |
| | | | | | | | by 300 nM CCI alone |
| | | | | | | | @ 24 h |
| LGALS3 | AB006780 | lectin, galactoside-binding, | 51.40 | 117.90 | 75.81 | 69.86 | 2-fold downregulated |
| | | soluble, 3 (galectin 3) | | | | | by 300 nM CCI alone |
| | | | | | | | @ 24 h |
| UNK_J04178 | J04178 | Human abnormal beta- | 1.85 | 16.24 | 23.19 | 16.38 | 2-fold downregulated |
| | | hexosaminidase alpha chain | | | | | by 300 nM CCI alone |
| | | (HEXA) mRNA, partial cds | | | | | @ 24 h |
| RNASE2 | X55988 | ribonuclease, RNase A family, | 11.30 | 20.81 | 36.24 | 57.24 | 2-fold downregulated |
| | | 2(liver, eosinophil-derived | | | | | by 300 nM CCI alone |
| | | neurotoxin) | | | | | @ 24 h |
| LILRB3 | AF025533 | leukocyte immunoglobulin-like | 18.25 | 34.14 | 39.62 | 59.90 | 2-fold downregulated |
| | | receptor, subfamily B (with | | | | | by 300 nM CCI alone |
| | | TM and ITIM domains), | | | | | @ 24 h AND 2-fold |
| | | member 3 | | | | | induced by PHA, 2-fold |
| | | | | | | | downregulated by 300 nM |
| | | | | | | | CCI |
| PAI2 | Y00630 | plasminogen activator | 84.55 | 287.95 | 160.10 | 33.95 | 2-fold downregulated |
| | | inhibitor, type II (arginine- | | | | | by 300 nM CCI alone |
| | | serpin) | | | | | @ 24 h AND 2-fold |
| | | | | | | | induced by PHA, 2-fold |
| | | | | | | | downregulated by 300 nM |
| | | | | | | | CCI |
| GRO2 | M36820 | GRO2 oncogene | 19.55 | 66.81 | 45.10 | 11.67 | 2-fold downregulated |
| | | | | | | | by 300 nM CCI alone |
| | | | | | | | @ 24 h |
| FCGR1A | M63835 | Fc fragment of IgG, high | 4.05 | 8.24 | 13.95 | 19.95 | 2-fold downregulated |
| | | affinity Ia, receptor for (CD64) | | | | | by 300 nM CCI alone |
| | | | | | | | @ 24 h |
| CTSL | X12451 | cathepsin L | 38.45 | 172.52 | 89.29 | 28.38 | 2 fold induced by PHA, |
| | | | | | | | 2-fold downregulated |
| | | | | | | | by 300 nM CCI |
| IL1RN | X52015 | interleukin 1 receptor | 16.60 | 83.90 | 88.67 | 36.29 | 2-fold downregulated |
| | | antagonist | | | | | by 300 nM CCI alone |
| | | | | | | | @ 24 h AND 2-fold |
| | | | | | | | induced by PHA, 2-fold |
| | | | | | | | downregulated by 300 nM |
| | | | | | | | CCI |
| UNK_AL096744 | AL096744 | Homo sapiens mRNA; cDNA | 5.65 | 27.95 | 29.67 | 23.52 | 2-fold downregulated |
| | | DKFZp566H033 (from clone | | | | | by 300 nM CCI alone |
| | | DKFZp566H033) | | | | | @ 24 h |
| IL1B | M15330 | interleukin 1, beta | 18.45 | 83.10 | 87.24 | 12.43 | 2-fold downregulated |
| | | | | | | | by 300 nM CCI alone |
| | | | | | | | @ 24 h |
| FABP5 | M94856 | fatty acid binding protein 5 | 7.20 | 31.29 | 21.33 | 7.90 | 2-fold downregulated |
| | | (psoriasis-associated) | | | | | by 300 nM CCI alone |
| | | | | | | | @ 24 h |
| SCYA7 | X72308 | small inducible cytokine A7 | 5.20 | 24.00 | 13.57 | 5.19 | 2-fold downregulated |
| | | (monocyte chemotactic protein 3) | | | | | by 300 nM CCI alone |
| | | | | | | | @ 24 h AND 2-fold |
| | | | | | | | induced by PHA, 2-fold |
| | | | | | | | downregulated by 300 nM |
| | | | | | | | CCI |
| NCF1 | M55067 | neutrophil cytosolic factor 1 | 8.70 | 10.48 | 20.86 | 46.62 | 2-fold downregulated |
| | | (47 kD, chronic granulomatous | | | | | by 300 nM CCI alone |
| | | disease, autosomal 1) | | | | | @ 24 h |
| SELP | M25322 | selectin P (granule membrane | 4.40 | 15.67 | 16.81 | 15.10 | 2-fold downregulated |
| | | protein 140 kD, antigen CD62) | | | | | by 300 nM CCI alone |
| | | | | | | | @ 24 h |
| SCYA20 | U64197 | small inducible cytokine | 2.35 | 7.00 | 11.05 | 2.67 | 2-fold downregulated |
| | | subfamily A (Cys-Cys), | | | | | by 300 nM CCI alone |
| | | member 20 | | | | | @ 24 h |
| GRO1 | X54489 | GRO1 oncogene (melanoma | 2.40 | 24.38 | 18.24 | 3.48 | 2-fold downregulated |
| | | growth stimulating activity, | | | | | by 300 nM CCI alone |
| | | alpha) | | | | | @ 24 h |
| SCYA2 | M26683 | small inducible cytokine A2 | 14.75 | 131.95 | 68.71 | 10.52 | 2-fold downregulated |
| | | (monocyte chemotactic protein | | | | | by 300 nM CCI alone |
| | | 1, homologous to mouse Sigje) | | | | | @ 24 h |
| TUBB | X79535 | tubulin, beta polypeptide | 8.70 | 19.00 | 31.57 | 41.52 | 2 fold induced by PHA, |
| | | | | | | | 2-fold downregulated |
| | | | | | | | by 300 nM CCI |

C. Monitoring CCI-779 Drug Activities

The CCI-779 activity genes identified in the present invention can be used to monitor CCI-779 drug activities in a patient who is subject to a CCI-779 treatment. Peripheral blood samples can be isolated at different stages of the CCI-779 treatment. The expression profile of one or more CCI-779 activity genes in these peripheral blood samples can be determined and compared to a reference expression profile. A change in the gene expression profile indicates in vivo activities of CCI-779. In one embodiment, the patent has RCC or another solid tumor.

Numerous methods are available for detecting gene expression profiles. In one aspect, the expression profiles of CCI-779 activity genes are determined by measuring the levels of RNA transcripts of these genes in peripheral blood samples. Suitable methods for this purpose include, but are not limited to, RT-PCT, Northern Blot, in situ hybridization, slot-blotting, nuclease protection assay, and nucleic acid arrays. The peripheral blood samples can be, without limitation, whole blood samples or samples containing enriched PBMCs.

In one embodiment, RNA isolated from peripheral blood samples can be first amplified to cDNA or cRNA. The amplification can be specific or non-specific. Suitable amplification methods include, but are not limited to, reverse transcriptase PCR, isothermal amplification, ligase chain reaction, and Qbeta replicase. The amplified nucleic acid products can be detected or quantitated, for instance, through hybridization to labeled probes.

Amplification primers and hybridization probes for a CCI-779 activity gene can be prepared from the gene sequence or its corresponding CPS using numerous methods. Gene sequences suitable for this purpose include, but are not limited to, exons, introns, or the 3′ or 5′ untranslated regions, or any combination thereof. In one embodiment, probes/primers are designed based on the sequence in or near the 3′ protein-coding region of a CCI-779 activity gene. For instance, the nucleotide sequence encoding the last 100 to 300 amino acid residues in the C-terminus region of the CCI-779 activity gene product can be selected to design probes or primers. Where a CCI-779 activity gene is a hypothetical or putative gene whose expression is supported only by EST or mRNA data, or where the genomic location(s) of a CCI-779 activity gene has not been determined or the gene may correspond to multiple genomic-loci, the probes/primers for the gene can be designed based on the corresponding CPS, or the oligonucleotide probes of the corresponding qualifier. Table 5 lists example sequences that are useful for designing probes/primers for detecting the expression profiles of CCI-779 activity genes.

The length of the probes/primers can be selected to achieve the desired hybridization or amplification effect. For instance, each probe can comprise at least 15, 20, 25, 50, 75, 100, 125, 150, 175, 200, 225, 250, 275, 300, 325, 350, 400 or more nucleotides. In one embodiment, each probe/primer has relatively high sequence complexity and does not have any ambiguous residue (undetermined “n” residues). In another embodiment, the probes/primers can hybridize to the target gene, such as its RNA transcripts or the complements thereof, under stringent or highly stringent conditions.

As used herein, “stringent conditions” are at least as stringent as, for example, conditions G-L shown in Table 7. “Highly stringent conditions” are at least as stringent as conditions A-F shown in Table 7. As used in Table 7, hybridization is carried out under the hybridization conditions (Hybridization Temperature and Buffer) for about four hours, followed by two 20-minute washes under the corresponding wash conditions (Wash Temp. and Buffer).

TABLE 7
Stringency Conditions

| Stringency | Polynucleotide | Hybrid | Hybridization | Wash Temp. |
|---|---|---|---|---|
| Condition | Hybrid | Length (bp)¹ | Temperature and Buffer^{H} | and Buffer^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{H}SSPE (1xSSPE is 0.15 M NaCl, 10 mM NaH₂PO₄, and 1.25 mM EDTA, pH 7.4) can be substituted for SSC (1xSSC is 0.15 M NaCl and 15 mM sodium citrate) in the hybridization and wash buffers. | | | | |
| T_{B}* − T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between | | | | |
| # 18 and 49 base pairs in length, Tₘ(° C.) = 81.5 + 16.6(log₁₀Na⁺) + 0.41(% G + C) − (600/N), where N is the number of bases in the hybrid, and Na⁺ is the molar concentration of sodium ions in the hybridization buffer (Na⁺ for 1xSSC = 0.165 M). | | | | |

In one embodiment, the probes/primers for a CCI-779 activity gene are selected from regions which significantly diverge from the sequences of other genes. Such regions can be determined by checking the probe/primer sequences against a human genome sequence database, such as the Entrez database at the NCBI. One algorithm suitable for this purpose is the BLAST algorithm. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence to increase the cumulative alignment score. Cumulative scores are calculated using, for nucleotide sequences, the parameters M (reward score for a pair of matching residues; always >0) and N (penalty score for mismatching residues; always <0). The BLAST algorithm parameters W, T, and X determine the sensitivity and speed of the alignment. These parameters can be adjusted for different purposes, as appreciated by one of ordinary skill in the art.

In one aspect, quantitative RT-PCR (such as TaqMan, ABI) is used for detecting and comparing the peripheral blood expression profiles of CCI-779 activity-genes. Quantitative RT-PCR involves reverse transcription (RT) of RNA to cDNA followed by relative quantitative PCR (RT-PCR).

In PCR, the number of molecules of the amplified target DNA increases by a factor approaching two with every cycle of the reaction until some reagent becomes limiting. Thereafter, the rate of amplification becomes increasingly diminished until there is not an increase in the amplified target between cycles. If one plots a graph on which the cycle number is on the X axis and the log of the concentration of the amplified target DNA is on the Y axis, one observes that a curved line of characteristic shape is formed by connecting the plotted points. Beginning with the first cycle, the slope of the line is positive and constant. This is said to be the linear portion of the curve. After some reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived may be determined for the respective tissues or cells. This direct proportionality between the concentration of the PCR products and the relative mRNA abundances is true in the linear range portion of the PCR reaction.

The final concentration of the target DNA in the plateau portion of the curve is determined by the availability of reagents in the reaction mix and is independent of the original concentration of target DNA. Therefore, the sampling and quantifying of the amplified PCR products can be carried out when the PCR reactions are in the linear portion of their curves. In addition, relative concentrations of the amplifiable cDNAs can be normalized to some independent standard, which may be based on either internally existing RNA species or externally introduced RNA species. The abundance of a particular mRNA species may also be determined relative to the average abundance of all mRNA species in the sample.

In one embodiment, the PCR amplification utilizes internal PCR standards that are approximately as abundant as the target. This strategy is effective if the products of the PCR amplifications are sampled during their linear phases. If the products are sampled when the reactions are approaching the plateau phase, then the less abundant product may become relatively over-represented. Comparisons of relative abundances made for many different RNA samples, such as is the case when examining RNA samples for differential expression, may become distorted in such a way as to make differences in relative abundances of RNAs appear less than they actually are. This can be improved if the internal standard is much more abundant than the target. If the internal standard is more abundant than the target, then direct linear comparisons may be made between RNA samples.

A problem inherent in clinical samples is that they are of variable quantity or quality. This problem can be overcome if the RT-PCR is performed as a relative quantitative RT-PCR with an internal standard in which the internal standard is an amplifiable cDNA fragment that is larger than the target cDNA fragment and in which the abundance of the mRNA encoding the internal standard is roughly 5-100 times higher than the mRNA encoding the target. This assay measures relative abundance, not absolute abundance of the respective mRNA species.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target cDNA fragment. In addition, the reverse transcriptase products of each RNA population isolated from the various samples can be normalized for equal concentrations of amplifiable cDNAs. While empirical determination of the linear range of the amplification curve and normalization of cDNA preparations are tedious and time-consuming processes, the resulting RT-PCR assays may, in certain cases, be superior to those derived from a relative quantitative RT-PCR with an internal standard.

Nucleic acid arrays can also be used to detect and compare the expression patterns of CCI-779 activity genes in peripheral blood samples isolated at different CCI-779 treatment stages. Probes suitable for detecting the CCI-779 activity genes can be stably attached to known discrete regions on a support substrate. These probes maintain their positions relative to the respective discrete regions during hybridization and subsequent washes. Construction of nucleic acid arrays is well known in the art. Suitable substrates for making nucleic acid arrays include, but are not limited to, glasses, silica, ceramics, nylons, quartz wafers, gels, metals, papers, beads, tubes, fibers, films, membranes, column matrixes, or microtiter plate wells.

A nucleic acid array of the present invention can comprise at least 2, 5, 10, 15, 20, 25, 30, 35, 40, 45, 50, 60, 70, 80, 90, 100, 150, 200, 250, or more different polynucleotide probes, each different probe capable of hybridizing to a different respective CCI-779 activity gene. Multiple probes for the same gene can be used on a single nucleic acid array. Examples of probes suitable for this invention are listed in the Qualifier Table. Probes for other drug activities genes can also be included in the nucleic acid arrays of this invention. The probe density on the array can be in any range. For instance, the density may be 50, 100, 200, 300, 400, 500, or more probes/cm².

In one embodiment, a substantial portion of all polynucleotide probes on a nucleic acid array of the present invention are probes for CCI-779 or other drug activity genes. For instance, at least 10%, 20%, 30%, 40%, 50%, or more of all probes on the nucleic acid array can hybridize under stringent or nucleic acid array hybridization conditions to RNA transcripts, or the complements thereof, of drug activity genes.

In another embodiment, nuclease protection assays are used to quantify RNAs derived from the peripheral blood samples. There are many different versions of nuclease protection assays. The common characteristic of these nuclease protection assays is that they involve hybridization of an antisense nucleic acid with the RNA to be quantified. The resulting hybrid double-stranded molecule is then treated with a nuclease which digests single-stranded nucleic acids more efficiently than double-stranded molecules. The amount of antisense nucleic acid that survives digestion is a measure of the amount of the target RNA species to be quantified. An example of nuclease protection assays is the RNase protection assay manufactured by Ambion, Inc. (Austin, Tex.).

In another aspect, the peripheral blood expression profiles of CCI-779 activity genes are determined by measuring the levels of polypeptides encoded by these genes. Methods suitable for this purpose include, but are not limited to, immunoassays such as ELISA, RIA, FACS, dot blot, Western Blot, immunohistochemistry, and antibody-based radioimaging. Other methods such as 2-dimensional SDS-polyacrylamide gel electrophoresis can also be used.

One exemplary method suitable for detecting the levels of target proteins in peripheral blood samples is ELISA. In an exemplifying ELISA, antibodies capable of binding to the target proteins encoded by one or more CCI-779 activity genes are immobilized onto a selected surface exhibiting protein affinity, such as wells in a polystyrene or polyvinylchloride microtiter plate. Then, peripheral blood samples to be tested are added to the wells. After binding and washing to remove non-specifically bound immunocomplexes, the bound antigen(s) can be detected. Detection can be achieved by the addition of a second antibody which is specific for the target proteins and is linked to a detectable label. Detection may also be achieved by the addition of a second antibody, followed by the addition of a third antibody that has binding affinity for the second antibody, with the third antibody being linked to a detectable label. Before being added to the microtiter plate, cells in the peripheral blood samples can be lysed using various methods known in the art. Proper extraction procedures can be used to separate the target proteins from potentially interfering substances.

In another exemplifying ELISA, the peripheral blood samples suspected of containing the target proteins are immobilized onto the well surface and then contacted with the antibodies of the invention. After binding and washing to remove non-specifically bound immunocomplexes, the bound antigen is detected. Where the initial antibodies are linked to a detectable label, the immunocomplexes can be detected directly. The immunocomplexes can also be detected using a second antibody that has binding affinity for the first antibody, with the second antibody being linked to a detectable label.

Another exemplary ELISA involves the use of antibody competition in the detection. In this ELISA, the target proteins are immobilized on the well surface. The labeled antibodies are added to the well, allowed to bind to the target proteins, and detected by means of their labels. The amount of the target proteins in an unknown sample is then determined by mixing the sample with the labeled antibodies before or during incubation with coated wells. The presence of the target proteins in the unknown sample acts to reduce the amount of antibody available for binding to the well and thus reduces the ultimate signal.

Different ELISA formats can have certain features in common, such as coating, incubating or binding, washing to remove non-specifically bound species, and detecting the bound immunocomplexes. For instance, in coating a plate with either antigen or antibody, the wells of the plate can be incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test samples. Examples of these nonspecific proteins include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means can also be used. After binding of a protein or antibody to the well, coating with a non-reactive material to reduce background, and washing to remove unbound material, the immobilizing surface is contacted with the control or clinical or biological sample to be tested under conditions effective to allow immunocomplex (antigen/antibody) formation. These conditions may include, for example, diluting the antigens and antibodies with solutions such as BSA, bovine gamma globulin (BGG) and phosphate buffered saline (PBS)/Tween and incubating the antibodies and antigens at room temperature for about 1 to 4 hours or at 4° C. overnight. Detection of the immunocomplex then requires a labeled secondary binding ligand or antibody, or a secondary binding ligand or antibody in conjunction with a labeled tertiary antibody or third binding ligand.

Following all incubation steps in an ELISA, the contacted surface can be washed so as to remove non-complexed material. For instance, the surface may be washed with a solution such as PBS/Tween, or borate buffer. Following the formation of specific immunocomplexes between the test sample and the originally bound material, and subsequent washing, the occurrence of the amount of immunocomplexes can be determined.

To provide a detection means, the second or third antibody can have an associated label. In one embodiment, the label is an enzyme that generates color development upon incubating with an appropriate chromogenic substrate. Thus, for example, one may contact and incubate the first or second immunocomplex with a urease, glucose oxidase, alkaline phosphatase or hydrogen peroxidase-conjugated antibody for a period of time and under conditions that favor the development of further immunocomplex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl)-benzthiazoline-6-sulfonic acid (ABTS) and H₂O₂, in the case of peroxidase as the enzyme label. Quantitation can be achieved by measuring the degree of color generation, e.g., using a spectrophotometer.

Another method suitable for this invention is RIA (radioimmunoassay). An exemplary RIA is based on the competition between radiolabeled-polypeptides and unlabeled polypeptides for binding to a limited quantity of antibodies. Suitable radiolabels include, but are not limited to, I¹²⁵ In one embodiment, a fixed concentration of I¹²⁵-labeled polypeptide is incubated with a series of dilution of an antibody specific to the polypeptide. When the unlabeled polypeptide is added to the system, the amount of the I¹²⁵-polypeptide that binds to the antibody is decreased. A standard curve can therefore be constructed to represent the amount of antibody-bound I¹²⁵-polypeptide as a function of the concentration of the unlabeled polypeptide. From this standard curve, the concentration of the polypeptide in unknown samples can be determined.

Suitable antibodies for this invention include, but are not limited to, polyclonal antibodies, monoclonal antibodies, chimeric antibodies, humanized antibodies, single chain antibodies, Fab fragments, or fragments produced by a Fab expression library. Methods for making these antibodies are well known in the art.

In one embodiment, the antibodies of the present invention can bind to the corresponding CCI-779 activity gene products or other desired antigens with a binding affinity constant Kₐ of at least 10⁴ M⁻¹,10⁵ M⁻¹, 10⁶M⁻¹,10⁷ M⁻¹,10⁸ M⁻¹, or more.

The antibodies of this invention can be labeled with one or more detectable moieties to allow for detection of antibody-antigen complexes. The detectable moieties can include compositions detectable by spectroscopic, enzymatic, photochemical, biochemical, bioelectronic, immunochemical, electrical, optical or chemical means. The detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

Moreover, the levels of polypeptides in peripheral blood samples can be determined by detecting the biological activities associated with the polypeptides. If a biological function/activity of a polypeptide is known, suitable in vitro bioassays can be designed to evaluate the biological function/activity, which in turn can be used to determine the amount of the polypeptide in the sample.

Comparison between the expression profile of a patient of interest and a reference expression profile can be conducted manually or electronically. The reference expression profile can be a baseline expression profile representing gene expression in peripheral blood samples isolated prior to a drug treatment. The reference profile can also be an expression profile in peripheral blood samples isolated after initiation of the drug treatment. The reference expression profile can be determine using sample isolated from the patient of interest or other reference patient or patients. In many embodiments, the process or methodology that is used to determine the reference expression profile and the expression profile being compared is identical or comparable.

In one example, comparison is carried out by comparing each component in the expression profile of the patient of interest to the corresponding component in the reference expression profile(s). The component can be the expression level of a drug activity gene, a ratio between the expression levels of two drug activity genes, or another measure capable of representing gene expression patterns. The expression level of a gene can be an absolute level, or a normalized or relative level. The difference between two corresponding components can be assessed by fold changes, absolute differences, or other suitable means.

Comparison between expression profiles can also be conducted using pattern recognition or comparison programs. In addition, the serial analysis of gene expression (SAGE) technology, the GEMTOOLS gene expression analysis program (Incyte Pharmaceuticals), the GeneCalling and Quantitative Expression Analysis technology (Curagen), and other suitable methods, programs or systems can be used.

Multiple drug activity genes can be used in the comparison of expression profiles. For instance, 2, 4, 6, 8, 10, 12, 14, 16, 18, 20, 30, 40, 50, or more drug activity genes can be used.

In one embodiment, the drug activity gene(s) used in the comparison can be selected to have relatively small p-values or large differential expression ratios. In one example, the drug activity genes used in the comparison have p-values (e.g., under an ANOVA analysis) of no greater than 0.05, 0.01, 0.001, 0.0005, 0.0001, or less. In another example, the expression level after or during a drug treatment is increased or decreased by at least 2-fold, 3-fold, 4-fold, 5-fold, or more over the baseline expression level.

In another embodiment, comparison of the expression profiles is performed electronically, such as by using a computer system. The computer system includes a processor coupled to a memory or storage medium which stores data representing the expression profiles being compared. In one example, the memory or storage medium is readable or rewritable. The stored expression data can be changed, retrieved, or otherwise manipulated. The memory can also store one or more programs capable of causing the processor to compare the expression profiles. In one embodiment, the processor is coupled to a nucleic acid array scanner which sends signals to the processor for analysis.

In vivo activities of other drugs can be similarly monitored using the drug activities genes of the present invention, as appreciated by those skilled in the art.

At least a subset of the drug activity genes of the present invention may be used as disease regression indicators. This subset of genes may be responsive to the effect of CCI-779 or other drugs on RCC or other diseases. In one example, CCI-779 or another drug may cause regression of tumors, and the decrease in tumor size or activity may cause a decreased immune response which is reflected in gene expression changes in the peripheral blood cells. Consequently, the drug activity genes of the present invention can be used as molecular markers for monitoring the efficacy of CCI-779 or other drugs for treating RCC or other diseases. In many cases, diseases amendable to the present invention include those whose regression can produce changed immune responses.

It should be understood that the above-described embodiments and the following examples are given by way of illustration, not limitation. Various changes and modifications within the scope of the present invention will become apparent to those skilled in the art from the present description.

### E. EXAMPLES

### Example 1

### Isolation of RNA and Preparation of Labeled Microarray Targets

PBMCs from the CCI-779 clinical trials were isolated from whole blood samples (8 mL) and collected into CPT tubes according to the standard procedure. PBMCs were purified over Ficoll gradients, washed two times with PBS and counted. Total RNA was isolated from PBMC pellets using the RNeasy mini kit (Qiagen, Valencia, Calif.). Labeled target for oligonucleotide arrays was prepared using a modification of the procedure described in Lockhart, et al., Nature Biotechnology 14: 1675-80 (1996). 2 μg total RNA was converted to cDNA by priming with an oligo-dT primer containing a T7 DNA polymerase promoter at the 5′ end. The cDNA was used as the template for in vitro transcription using a T7 DNA polymerase kit (Ambion, Woodlands, Tex.) and biotinylated CTP and UTP (Enzo). Labeled cRNA was fragmented in 40 mM Tris-acetate pH 8.0, 100 mM KOAc, 30 mM MgOAc for 35 minutes at 94° C. in a final volume of 40 PI.

### Example 2

### Hybridization to Affymetrix Microarrays and Detection of Fluorescence

Individual samples were hybridized to HgU95A genechip (Affymetrix). No samples were pooled. 110 RCC patients were involved in the study. Each patient received 25, 75, or 250 mg of CCI-779 once weekly through intravenous infusion. Blood samples were collected immediately before the first CCI-779 infusion, eight weeks after the first infusion, and sixteen weeks after the first infusion.

10 μg of labeled target was diluted in 1×MES buffer with 100 μg/ml herring sperm DNA and 50 μg/ml acetylated BSA. To normalize arrays to each other and to estimate the sensitivity of the oligonucleotide arrays, in vitro synthesized transcripts of 11 bacterial genes were included in each hybridization reaction as described in Hill et al., Science, 290: 809-812 (2000). The abundance of these transcripts ranged from 1:300,000 (3 ppm) to 1:1000 (1000 ppm) stated in terms of the number of control transcripts per total transcripts. As determined by the signal response from these control transcripts, the sensitivity of detection of the arrays ranged between about 1:300,000 and 1:100,000 copies/million. Labeled probes were denatured at 99° C. for 5 minutes and then 45° C. for 5 minutes, and hybridized to oligonucleotide arrays comprised of over 12,500 human gene probes (HgU95A, Affymetrix). Arrays were hybridized for 16 hours at 45° C. The hybridization buffer was comprised of 100 mM MES, 1 M [Na⁺], 20 MM EDTA, and 0.01% Tween 20. After hybridization, the cartridges were washed extensively with non-stringent wash buffer (6×SSPET), such as three 1.0-minute washes at room temperature. These hybridization and wash conditions are herein collectively referred to as “nucleic acid array hybridization conditions.” The washed cartridges were then stained with phycoerythrin coupled to streptavidin.

12×MES stock contains 1.22 M MES and 0.89 M [Na+]. For 1000 ml, the stock can be prepared by mixing 70.4 g MES free acid monohydrate, 193.3 g MES sodium salt and 800 ml of molecular biology grade water, and adjusting volume to 1000 ml. The pH should be between 6.5 and 6.7.2×hybridization buffer can be prepared by mixing 8.3 mL of 12×MES stock, 17.7 mL of 5 M NaCl, 4.0 mL of 0.5 M EDTA,0.1 mL of 10% Tween 20 and 19.9 mL of water. 6×SSPET contains 0.9 M NaCl, 60 mM NaH₂PO₄, 6 mM EDTA, pH 7.4, and 0.005% Triton X-100. In some cases, the wash buffer can be replaced with a more stringent wash buffer. 1000 ml stringent wash buffer can be prepared by mixing 83.3 mL of 12×MES stock, 5.2 mL of 5 M NaCl, 1.0 mL of 10% Tween 20 and 910.5 mL of water.

### Example 3

### Gene Expression Data Analysis

Data analysis was performed on raw fluorescent intensity values using GENECHIP 3.2 software (Affymetrix). GeneChip 3.2 software uses an algorithm to calculate the likelihood as to whether a gene is “absent” or “present” as well as a specific hybridization intensity value or “average difference” for each transcript represented on the array. The algorithms used in these calculations are described in the Affymetrix GeneChip Analysis Suite User Guide (Affymetrix). The “average difference” for each transcript was normalized to “frequency” values according to the procedures of Hill et al., Science, 290: 809-812 (2000). This was accomplished by referring the average difference values on each chip to a calibration curve constructed from the average difference values for the 11 control transcripts with known abundance that were spiked into each hybridization solution. This process also served to normalize between arrays.

Specific transcripts were evaluated further if they met the following criteria. First, genes that were designated “absent” by the GENECHIP 3.2 software in all samples were excluded from the analysis. Second, in comparisons of transcript levels between arrays, a gene was required to be present in at least one of the arrays. Third, for comparisons of transcript levels between groups, an ANOVA was applied to identify a subset of transcripts that had a significant (p<0.05) differences in frequency values.

A ANOVA was used to compare PBMC expression profiles measured immediately before the first infusion of CCI-779, to PBMC expression profiles measured at eight and sixteen weeks after the first infusion. In the comparisons, a p value <0.05 was used to indicate statistical significance. Transcripts that were altered, on average, by 2-fold or more between any two time points can be selected.

### Example 4

### Ex Vivo Assays

Drug modulable transcripts in the surrogate tissue, such as peripheral blood, can provide early evidence of drug exposure in vivo. PBMC ex vivo assays that mimic peripheral blood gene expression in disease states as well as the in vivo response to drug treatments can further validate the surrogate markers identified in in vivo studies. In addition, ex vivo assays that overlay with disease-associated biomarkers may provide evidence for markers of drug-dependent disease amelioration.

Many factors should be considered in designing suitable ex vivo assays. These factors include, but are not limited to, PBMC seeding density, culture media and other conditions, vehicle control, dose response, time course, and dose schedule (e.g., preincubation, coincubation, or postincubation).

In one embodiment, phytohemagglutinin (PHA) was used to stimulate gene expression in PBMCs. The genes whose expression can be stimulated by PHA in PBMCs had a substantial overlap with disease-associated transcripts in RCC PBMCs that appear to result from T-cell activation. In some cases, PHA-stimulated PBMCs mimicked anti-CD3/anti-CD28 stimulated T-cell profiles in BioExpress. In addition, CCI-779 inhibited certain PHA-inducible transcripts in a dose-dependent manner. The inhibition by CCI-779 appeared to be specific, rather than global. In one example, CTSL, SCYA2, FABP5, SCYA7, ATP2B1, and IL1R1 genes were directly repressed by 300 nm CCI-779 in PHA-stimulated PBMCs in an ex vivo assay. Databases of disease-associated profiles and ex vivo activation signatures can be created to identity most appropriate culture conditions for identification of drug modulated biomarkers in specific disease settings.

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

1. A method comprising comparing an expression profile of at least one gene in a peripheral blood sample of a patient to a reference expression profile of said at least one gene, wherein said at least one gene is differentially expressed in peripheral blood mononuclear cells (PBMCs) of patients who have a non-blood disease and are subject to a drug therapy as compared to PBMCs isolated from said patients before said drug therapy, and wherein the patient has the non-blood disease and is being treated by said drug therapy.

2. The method according to claim 1, wherein said drug therapy is a CCI-779 therapy.

3. The method according to claim 2, wherein the non-blood disease is a solid tumor.

4. The method according to claim 3, wherein the solid tumor is RCC.

5. The method according to claim 2, wherein said at least one gene includes one or more genes selected from Table 5.

6. The method according to claim 2, wherein said at least one gene includes at least two genes selected from Table 5.

7. The method according to claim 1, wherein the peripheral blood sample is a whole blood sample.

8. The method according to claim 1, wherein the peripheral blood sample comprises enriched PBMCs.

9. The method according to claim 1, wherein the expression profile is determined by RT-PCR or immunoassays.

10. The method according to claim 1, wherein the reference expression profile is an average expression profile of said at least one gene in peripheral blood samples isolated from said patients before said drug therapy.

11. The method according to claim 1, wherein the reference expression profile is an expression profile of said at least one gene in a reference peripheral blood sample isolated from the patient before said drug therapy.

12. The method according to claim 1, wherein said at least one gene includes one or more genes which are over-expressed or under-expressed in PBMCs of patients who have the non-blood disease as compared to PBMCs of humans who do not have the non-blood disease, and wherein said drug therapy is capable of down-regulating or up-regulating expression of said one or more genes in PBMCs of patients who have the non-blood disease.

13. The method according to claim 1, wherein said at least one gene includes one or more genes whose expression in PBMCs is capable of being increased or reduced by a phytohemagglutinin treatment, and wherein said drug therapy is capable of down-regulating or up-regulating expression of said one or more genes in phytohemagglutinin-treated PBMCs.

14. The method according to claim 1, wherein RNA transcripts of said at least one gene are capable of hybridizing under stringent or nucleic acid array hybridization conditions to one or more qualifiers selected from the Qualifier Table.

15. The method according to claim 14, wherein said drug therapy is a CCI-779 therapy.

16. The method according to claim 15, wherein the non-blood disease is RCC.

17. A method for identifying drug activity genes, comprising:
detecting an expression profile of genes in peripheral blood samples of patients who have a non-blood disease and are subject to a drug therapy; and
comparing said expression profile to a baseline expression profile of said genes in peripheral blood samples isolated from said patients before said drug therapy so as to identify drug activity genes whose expression levels in peripheral blood samples can be modulated by said drug therapy.

18. A kit comprising a plurality of polynucleotides, wherein each of said polynucleotides is capable of hybridizing under stringent or nucleic acid array hybridization conditions to an RNA transcript, or the complement thereof, of a different respective gene selected from Table 5.

19. A kit comprising a plurality of antibodies, wherein each of said antibodies is capable of binding to a polypeptide encoded by a different respective gene selected from Table 5.

20. A nucleic acid array comprising polynucleotide probes, wherein a substantial portion of all polypeptide probes on the nucleic acid array can hybridize under stringent or nucleic acid array hybridization conditions to RNA transcripts, or the complements thereof, of genes selected from Table 5.
